SEQUENCE LISTING (20 sequences)

SEQ ID NO: 1 - protein, 110 aa, Artificial
  Ranibizumab Light Chain Variable Domain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 2 - protein, 158 aa, Artificial
  Ranibizumab Heavy Chain Variable Domain
SGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 3 - protein, 214 aa, Artificial
  Ranibizumab Light Chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 235 aa, Artificial
  Ranibizumab Heavy Chain
EVQLVESGGGLVQPGGSLRLSCAASGYDFTHYGMETNWVRQAPGKGLEWVGWINTYTGEPTYAADFKRRFTFSLDTSKSTAYLQMETNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 22 aa, Pectobacterium brasiliense
MKYLLPTAAAGLLLLAAQPAMA

SEQ ID NO: 6 - protein, 21 aa, Escherichia coli
MKKTAIAIAVALAGFATVAQA

SEQ ID NO: 7 - protein, 23 aa, Escherichia coli
MKKNIAFLLASMFVFSIATNAYA

SEQ ID NO: 8 - protein, 28 aa, Bacillus velezensis
MFKFKKKFLVGLTAAFMSISMFSATASA

SEQ ID NO: 9 - protein, 21 aa, Artificial
  synthetic polypeptide
MKQSTAILALLPLLFTPVTKA

SEQ ID NO: 10 - protein, 22 aa, Escherichia coli
MMKRNILAVIVPALLVAGTANA

SEQ ID NO: 11 - protein, 21 aa, Escherichia coli
MKKSTLALVVMGIVASASVQA

SEQ ID NO: 12 - protein, 26 aa, Escherichia coli
MKIKTGARILALSALTTMMFSASALA

SEQ ID NO: 13 - protein, 21 aa, Escherichia coli
MKVKVLSLLVPALLVAGAANA

SEQ ID NO: 14 - protein, 20 aa, Escherichia coli
MKATKLVLGAVILGSTLLAG

SEQ ID NO: 15 - protein, 25 aa, Escherichia coli
MMITLRKLPLAVAVAAGVMSAQAMA

SEQ ID NO: 16 - protein, 20 aa, Escherichia coli
MRAKLLGIVLTTPIAISSFA

SEQ ID NO: 17 - protein, 23 aa, Escherichia coli
MSIQHFRVALIPFFAAFCLPVFA

SEQ ID NO: 18 - protein, 21 aa, Escherichia coli
MKQSTIALALLPLLFTPVTKA

SEQ ID NO: 19 - protein, 25 aa, Escherichia coli
MKVMRTTVATVVAATLSMSAFSVFA

SEQ ID NO: 20 - protein, 23 aa, Klebsiella pneumoniae
MKKSLVLKASVAVATLVPMLSFA

(19) United States
(12) Patent Application Publication
(10) Pub. No.: US 2020/0207812 A1
(43) Pub. Date: Jul. 2, 2020

(54) METHODS FOR PRODUCING RECOMBINANT PROTEINS

(71) Applicant: Coherus BioSciences, Inc., Redwood City, CA (US)

(72) Inventor: Robert Neal Williams, Mountain View, CA (US)

(73) Assignee: Coherus BioSciences, Inc., Redwood City, CA (US)

(21) Appl. No.: 16/704,776

(22) Filed: Dec. 5, 2019

Related U.S. Application Data
(60) Provisional application No. 62/776,880, filed on Dec. 7, 2018.

(51) Int. Cl. C07K 14/245; C12N 5/00; C12N 15/70

(52) U.S. Cl. CPC C07K 14/245; C12N 5/0068; A61K 45/06; C12N 2510/02; C12N 2527/00; C12N 15/70

(57) ABSTRACT

Provided herein are methods of producing a recombinant protein that include: providing a bacterium including a nucleic acid encoding a recombinant protein; and culturing the bacterium in a liquid culture medium including about 0.3 mM to about 300 mM Mg²⁺ under conditions sufficient for the production and release of the recombinant protein into the culture medium.

### CROSS-REFERENCE TO RELATED APPLICATIONS

This application claims priority to U.S. Provisional Patent Application Ser. No. 62/776,880, filed Dec. 7, 2018; the entire contents of which are herein incorporated by reference.

### TECHNICAL FIELD

This disclosure relates to methods of biotechnology and the manufacturing of recombinant proteins.

### BACKGROUND

Recombinant bacteria are used in many biotechnology protocols, including the production of recombinant proteins. Recombinant proteins are often used in the preparation of new therapeutic drug products. Current methods for producing recombinant proteins in bacteria result in the protein remaining within the bacteria (e.g., retained in the periplasm of the bacteria).

The periplasm is an oxidative space which promotes proper refolding of proteins (especially those with cystine residues). To harvest the protein, the bacteria must be separated from the culture medium and lysed or disrupted (either chemically and/or physically) to release the recombinant protein. Lysing and/or disrupting the bacteria also releases unwanted host cell proteins and potentially dangerous endotoxins, which must be purified away from the recombinant protein before it can used in a drug product. Thus, the current methods for recovering recombinant proteins possess limitations which the present invention solves.

### SUMMARY

The present disclosure is based, at least in part, on the discovery culturing recombinant bacteria in a liquid culture medium including about 0.3 mM to about 300 mM Mg²⁺, or about 0.07 g/L to about 72.0 g/L of a magnesium salt, results in a significant release of recombinant protein retained within the periplasmic space of the recombinant bacterium into the liquid culture medium. In view of this discovery, provided herein are methods of producing a recombinant protein that include: providing a recombinant bacterium including a nucleic acid encoding a recombinant protein; and culturing the recombinant bacterium in a liquid culture medium that includes about 0.3 mM to about 300 mM Mg²⁺, or about 0.07 g/L to about 72.0 g/L of a magnesium salt, under conditions sufficient for the production and release of a recombinant protein into the liquid culture medium.

Provided herein are methods of producing a recombinant protein that include: providing a recombinant bacterium including a nucleic acid encoding a recombinant protein; and culturing the recombinant bacterium in a liquid culture medium including about 0.3 mM to about 300 mM of Mg²⁺ under conditions sufficient for the production and release of the recombinant protein into the liquid culture medium. In some embodiments of any of the methods described herein, the liquid culture medium includes a magnesium salt. In some embodiments of any of the methods described herein, the magnesium salt is MgSO₄.

Some embodiments of any of the methods described herein further include recovering the recombinant protein from the liquid culture medium. In some embodiments of any of the methods described herein, the recovered recombinant protein is at least 95% pure.

Some embodiments of any of the methods described herein further include purifying the recovered recombinant protein.

Some embodiments of any of the methods described herein further include formulating the purified recombinant protein. In some embodiments of any of the methods described herein, the method does not include the performance of more than two chromatography steps. In some embodiments of any of the methods described herein, the method does not include physical or chemical disruption of the outer membrane of the recombinant bacterium.

In some embodiments of any of the methods described herein, the culturing is performed using a fermentor. In some embodiments of any of the methods described herein, the culturing is batch culturing. In some embodiments of any of the methods described herein, the culturing is fed batch culturing.

In some embodiments of any of the methods described herein, the culturing includes incubating the recombinant bacterium at a rotary agitation rate of about 80 revolutions per minute (RPM) to about 1000 RPM (e.g., about 100 RPM to about 850 RPM). In some embodiments of any of the methods described herein, the culturing is performed at about 30° C. to about 37° C. In some embodiments of any of the methods described herein, the recombinant bacterium is a Gram negative bacterium. In some embodiments of any of the methods described herein, the recombinant bacterium is selected from the group consisting of: K12 E. coli bacterial cell, a Yersinia bacterial cell, a BL21 E. coli bacterial cell, a 60E4 E. coli, an Acinetobacter bacterial cell, a Bordetlla bacterial cell, a Brucella bacterial cell, a Cyanobacter bacterial cell, an Enterobacter bacterial cell, a Helicobacter bacterial cell, a Klebsiella bacterial cell, a Neisseria bacterial cell, a Pasteurella bacterial cell, a Pseudomonas bacterial cell, a Salmonella bacterial cell, and a Shigella bacterial cell.

In some embodiments of any of the methods described herein, the nucleic acid encoding the recombinant protein is integrated into a chromosome of the recombinant bacterium. In some embodiments of any of the methods described herein, the nucleic acid encoding the recombinant protein is not integrated into a chromosome of the recombinant bacterium.

In some embodiments of any of the methods described herein, the recombinant protein is an antibody or an antigen-binding antibody fragment. In some embodiments of any of the recombinant proteins described herein, the recombinant protein is an antibody or an antigen-binding antibody fragment that specifically binds to human vascular endothelial growth factor A (VEGF-A). In some embodiments of any of the methods described herein, the antigen-binding antibody fragment is ranibizumab.

In some embodiments of any of the methods described herein, the nucleic acid encoding the recombinant protein is an expression vector. In some embodiments of any of the methods described herein, the nucleic acid encoding the recombinant protein includes a sequence encoding a bacterial signal sequence.

In some embodiments of any of the methods described herein, the liquid culture medium includes about 0.3 mM to about 300 mM Mg²⁺ (e.g., about 0.4 mM to about 41 mM Mg²⁺, about 0.4 mM to about 20 mM Mg²⁺, about 0.4 mM to about 10 mM Mg²⁺, or about 0.4 mM to about 5 mM Mg²⁺). In some embodiments of any of the methods described herein, the liquid culture medium includes about 0.3 mM Mg²⁺, about 0.92 mM Mg²⁺, about 1.41 mM Mg²⁺, about 2.70 mM Mg²⁺, about 5.41 mM Mg²⁺, about 9.57 mM Mg²⁺, or about 300 mM Mg²⁺.

Also provided herein is a recombinant protein produced by any of the methods described herein. Also provided herein are compositions that include any of the recombinant proteins produced by any of the methods described herein.

Also provided herein are pharmaceutical compositions that include a therapeutically effective amount of any of the recombinant proteins described herein produced by any of the methods described herein. In some embodiments of any of the pharmaceutical compositions described herein, the recombinant protein is ranibizumab. In some embodiments of any of the pharmaceutical compositions described herein, the pharmaceutical composition includes about 5 mg/mL to about 10 mg/mL ranibizumab. Some embodiments of any of the pharmaceutical compositions described herein further include a tonicity agent, a buffer, a surfactant, and water for injection, and wherein the pharmaceutical composition has a pH of about 5 to about 6. Some embodiments of any of the pharmaceutical compositions described herein, the pharmaceutical composition includes α,α-trehalose dihydrate, a histidine buffer, polysorbate 20, and water for injection, and the pharmaceutical composition has a pH of about 5 to about 6.

Also provided herein are kits that include any of the pharmaceutical compositions described herein. Some embodiments of any of the kits described herein further include a sterile glass vial, where the pharmaceutical composition is disposed within the sterile glass vial. Some embodiments of any of the kits described herein further include a syringe, where the pharmaceutical compositions is disposed within the syringe.

Also provided herein are methods of treating a subject in need thereof that include administering to the subject a therapeutically effective amount of any of the pharmaceutical compositions described herein. In some embodiments of any of the methods of treating a subject described herein, the subject has been identified or diagnosed as having wet age-related macular degeneration, diabetic macular edema, or macular edema following retinal vein occlusion (e.g., branch retinal vein occlusion or central retinal vein occlusion).

As used herein, the word “a” before a noun represents one or more of the particular noun. For example the “a recombinant bacterial cell” represents “one or more recombinant bacterial cells.”

The term “bacterium” or “bacterial cell” means any cell from or derived from any bacterium (e.g., a Gram positive bacterium, or a Gram negative bacterium). Non-limiting examples of bacteria are described herein. Additional examples of bacteria are known in the art.

The term “recombinant bacterium” means a bacterium that contains a nucleic acid that is not naturally present in the bacterium. For example, the nucleic acid that is not naturally present in the bacterium can encode a recombinant protein (e.g., any of the exemplary recombinant proteins described herein) and/or can encode a selectable marker (e.g., any of the exemplary selectable markers described herein). The nucleic acid that is not naturally present in the cell can, e.g., be integrated into the genome of the bacterium. In other examples, the nucleic acid that is not naturally present in the cell is not integrated into the genome of the bacterium. For example, a nucleic acid that is not naturally present in the bacterium can be episomal.

The term “culturing” or “cell culturing,” as used herein, refers to the maintenance or proliferation of a bacterium (e.g., a recombinant bacterium) under a controlled set of physical conditions. Non-limiting examples of the physical conditions that can be used to culture a recombinant bacterium are described herein.

The term “fed-batch culturing,” as used herein refers to culturing, in a container or vessel (e.g., a fermentor), a plurality of bacteria that have been contacted with a liquid culture medium (e.g., any of the exemplary liquid culture media described herein) to obtain a recombinant protein production medium, wherein the culturing of the cells present in a container or vessel includes the periodic or continuous addition of a volume of liquid culture medium to the recombinant protein production medium without substantial or significant removal of the recombinant protein production medium. For example, non-substantial or insignificant removal of medium includes removal of a small amount of medium for monitoring and/or analysis of the culture or culture conditions. In some embodiments, fed-batch culturing comprises contacting recombinant bacteria capable of producing a recombinant protein with a liquid culture medium in a fed-batch bioreactor which includes the periodic or continuous addition of a volume of liquid culture medium to the fed-batch bioreactor without substantial or significant removal of the recombinant protein production medium. In some embodiments, the liquid culture medium added after initial preparation of a recombinant protein production medium (such later-added medium being understood in the art, and referred to herein, as a feed liquid culture medium) is the same as, or different from, the initial liquid culture medium first contacted with recombinant bacteria to initially obtain the recombinant protein production medium. In some examples of fed-batch culture, the later-added feed liquid culture medium is a concentrated liquid culture medium. Skilled practitioners will appreciate that, in some embodiments, a fermentor can be adapted for fed-batch culturing (e.g., adapted to be a fed-batch fermentor).

The term “liquid culture medium” comprises a fluid that (i) contains sufficient nutrients to allow a cell (e.g., a bacterial cell) to grow or proliferate in vitro; and (ii) has not yet been placed in contact with, combined with, or added to, such cells. In some embodiments, a liquid culture medium is a minimal medium (e.g., a medium containing only inorganic salts, a carbon source, and water). Non-limiting examples of a liquid culture medium are described herein. Additional examples of a liquid culture medium are described in the art and are commercially available. In some examples, a liquid culture medium can be intended for addition, as a feed liquid culture medium, to an already prepared recombinant protein production medium (as defined below).

The term “recombinant protein production medium,” as used herein, comprises a fluid suitable for producing a recombinant protein obtained by contacting a liquid culture medium with cells (e.g., recombinant bacteria) comprising a nucleic acid encoding the recombinant protein. For purposes herein, it should be understood that a “liquid culture medium” differs from a “recombinant protein production medium” in that the latter, for example, can contain products of cellular metabolism as well as the recombinant protein being produced by the cells (e.g., recombinant bacteria). Moreover, cellular metabolism occurring in the recombinant protein production medium consumes energy in the liquid culture medium, and produces metabolites. A recombinant protein production medium can be generated in a culture of recombinant bacteria during the recombinant protein production phase of the culture. In some aspects, a recombinant protein production medium does not include phosphate.

The terms “magnesium salt,” “Mg++ salt,” “Mg⁺² salt,” and “Mg salt,” as used herein, refers to any magnesium ion containing compound including, but not limited to, MgCl₂, MgSO₄, Mg pidolate, MgHPO₄, MgBr₂, and MgCrO₄. In some embodiments, the magnesium salt is provided as a solid (e.g. granules or a powder). In other embodiments, the magnesium salt is provided in a liquid solution (e.g. dissolved into a solvent such as water). In some embodiments, the magnesium salt is suitable for addition to a medium, such as a liquid culture medium or a recombinant protein production medium.

The term “agitating” means stirring or otherwise moving a portion of liquid culture medium and/or recombinant protein culture medium in a bioreactor or vessel (e.g., a fermentor). Agitating is performed in order to increase the dissolved O₂ concentration in the liquid culture medium and/or recombinant protein culture medium in a bioreactor or vessel; (e.g., a fermentor). In some embodiments, agitation is performed using any art known method, e.g., an instrument or propeller. Exemplary devices and methods used to perform agitation of a portion of the liquid culture medium or the recombinant protein culture medium in a bioreactor or vessel (e.g., a fermentor) are known in the art.

The term “immunoglobulin,” as used herein comprises a polypeptide containing an amino acid sequence of at least 15 amino acids (e.g., at least 20, 30, 40, 50, 60, 70, 80, 90, or 100 amino acids) of an immunoglobulin protein (e.g., a variable domain sequence, a framework sequence, or a constant domain sequence). In some embodiments, the immunoglobulin comprises at least 15 amino acids of a light chain immunoglobulin and at least 15 amino acids of a heavy chain immunoglobulin. In some embodiments, the immunoglobulin is an isolated antibody (e.g., an IgG, IgE, IgD, IgA, or IgM). In some embodiments, the immunoglobulin is a subclass of IgG (e.g., IgG1, IgG2, IgG3, or IgG4). In some embodiments, the immunoglobulin is a mouse, chimeric, humanized, or human antibody. In some embodiments, the immunoglobulin is an antibody fragment, e.g., a Fab fragment, a F(ab′)₂ fragment, or a scFv fragment. In some embodiments, the immunoglobulin is a bi-specific antibody or a tri-specific antibody, or a dimer, trimer, or multimer antibody, or a diabody, an Affibody®, or a Nanobody®. In some embodiments, the immunoglobulin is an engineered protein containing at least one immunoglobulin domain (e.g., a fusion protein). Non-limiting examples of immunoglobulins are described herein and additional examples of immunoglobulins are described in the art.

The term “capturing,” as used herein, refers to partially purifying or isolating and concentrating a recombinant protein from one or more other components present in a recombinant protein production medium or a diluted recombinant protein production medium. In some embodiments, capturing is performed using a resin that binds a recombinant protein (e.g., through the use of affinity chromatography). Non-limiting methods for capturing a recombinant protein from a recombinant protein production medium or diluted recombinant protein production medium are described herein and in the art. In some embodiments, the recombinant protein is captured from a recombinant protein production medium or a diluted recombinant protein production medium using at least one chromatography column (e.g., any of the chromatography columns described herein).

The term “recovering,” as used herein, comprises at least partially purifying or isolating (e.g., at least or about 4% (e.g., at least or about 5%, 10%, 12%, 15%, 20%, 25%, 30%, 35%, 40%, 45%, 50%, 55%, 60%, 65%, 70%, 75%, 80%, 85%, 90% or 95%) a recombinant protein from one or more components present in the recombinant protein production medium (e.g., recombinant bacterial cells, or one or more other components (e.g., DNA, RNA, or other proteins) present in a recombinant bacterial cell lysate). Non-limiting methods for recovering a recombinant protein from a recombinant protein production medium or from a recombinant bacterial cell lysate are described herein and are disclosed in the art.

The term “purifying,” as used herein, comprises isolating a recombinant protein from one or more other impurities (e.g., bulk impurities) or components present in a fluid containing a recombinant protein (e.g., a recombinant protein production medium). In some embodiments, purifying comprises isolating a recombinant protein from liquid culture medium proteins or one or more other components (e.g., DNA, RNA, other proteins, endotoxins, viruses, etc.) present in or secreted from a bacterial cell. In some embodiments, purification is performed using a resin that binds either a recombinant protein or contaminants. Non-limiting examples include affinity chromatography, hydrophobic interaction chromatography, anion or cation exchange chromatography, or molecular sieve chromatography. In some embodiments, a recombinant protein is purified from a fluid containing the recombinant protein (e.g., a recombinant protein production medium) using at least one chromatography column (e.g., any of the chromatography columns described herein).

The term “polishing,” as used herein, refers to a step performed to remove remaining trace or small amounts of contaminants or impurities from a fluid containing a recombinant protein that is close to a final desired purity. In a non-limiting example, polishing is performed by passing a fluid containing the recombinant protein through a chromatographic column(s) or a membrane absorber(s) that selectively binds to either the recombinant protein or small amounts of contaminants or impurities present in a fluid containing the recombinant protein. In such an example, the eluate/filtrate of the chromatographic column(s) or the membrane absorber(s) contains the recombinant protein.

The term “filtering,” as used herein, refers to the removal of at least part of undesired biological contaminants (e.g., yeast cells, viruses, or bacteria) and/or particulate matter (e.g., precipitated proteins) from a liquid (e.g., a recombinant protein production medium or other fluid).

The term “secreted protein” or “released protein,” as used herein comprises a protein (e.g., a recombinant protein) that originally contained at least one secretion signal sequence when it is translated within a bacterial cell, and through, at least in part, enzymatic cleavage of the secretion signal sequence in the bacterial cell, is secreted into the extracellular space. In some embodiments, the extracellular space is a liquid culture medium and/or a recombinant protein production medium. Skilled practitioners will appreciate that a “secreted” protein need not dissociate entirely from the bacterium outer membrane to be considered a secreted protein. As used herein, a secreted recombinant protein does not refer to a protein retained in the periplasm of a recombinant bacterium.

The term “fed-batch bioreactor,” as used herein, refers to a bioreactor containing, or suitable for containing, a plurality of bacteria (e.g., recombinant bacteria) in a recombinant protein production medium, where the cell culture present in the bioreactor includes the periodic or continuous addition of a feed liquid culture medium to the recombinant protein production medium without substantial or significant removal of the recombinant protein production medium from the cell culture. In some embodiments, the feed liquid culture medium subsequently added to an already prepared recombinant protein production medium is the same as the liquid culture medium used to initially prepare the recombinant protein production medium. In some examples of fed-batch culture, the later-added feed liquid culture medium is a concentrated form of the initial liquid culture medium.

The term “yield” refers to a ratio of a total amount of recombinant protein obtained over a theoretical amount of recombinant protein that could be obtained (expressed as a percentage), based on the amount of starting materials.

The term “promoter” is a nucleic acid sequence that is operably linked to a nucleic acid sequence encoding a protein (e.g., a recombinant protein) that can increase the transcription of the nucleic acid sequence encoding the protein. In some aspects, a promoter is constitutive. In other aspects, a promoter is inducible. Non-limiting examples of promoters are described herein. Additional examples of promoters are known in the art.

The term “formulating,” as used herein, comprises processing a recombinant protein in a format that is compatible with its intended route of administration (e.g., intraarterial, intradermal, intramuscular, intravenous, intraperitoneal, subcutaneous, or oral) to a subject. In some aspects, formulating includes providing the recombinant protein in a dosage unit form (i.e., physically discrete units containing a predetermined quantity of recombinant protein for ease of administration and uniformity of dosage). In some aspects, formulating can include a sterile diluent (e.g., sterile water, saline, or water for injection), a fixed oil, polyethylene glycol, glycerin, propylene glycol, or other synthetic solvents, antibacterial or antifungal agents (e.g., benzyl alcohol, methyl parabens, chlorobutanol, phenol, ascorbic acid, thimerosal), antioxidants (e.g., ascorbic acid or sodium bisulfite), chelating agents (e.g., ethylenediaminetetraacetic acid), buffers (e.g., acetates, citrates, phosphates), isotonic agents (e.g., sugars, polyalcohols (e.g., mannitol or sorbitol), or salts (e.g., sodium chloride), or any combination thereof. Non-limiting examples of formulations of a recombinant protein are described herein.

Unless otherwise defined, all technical and scientific terms used herein have the same meanings as commonly understood by one of ordinary skill in the art to which this disclosure belongs. Methods and materials are described herein for use in the present disclosure; other, suitable methods and materials described in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### BRIEF DESCRIPTION OF DRAWINGS

FIG. 1 is an exemplary schematic representation of a genetic map of a pBR322:236715 plasmid (6418 basepairs (bp)). The vector includes a repressor of primer (ROP), an origin of replication (Ori_ColE1), an ampicillin promoter (P_Amp), an ampicillin resistance gene (MAmpicillin-r), a tetracycline resistance gene (M_Tetracycline-r) and a gene.

FIG. 2 is an exemplary schematic representation of a recombinant bacterial cell expressing ranibizumab.

FIG. 3 is a representative graph showing the periplasm titers of ranibizumab (mg/L) over time in recombinant bacteria cultures using liquid culture media including different concentrations of MgSO₄.7H₂O (0.21 g/L, 0.32 g/L, 0.64 g/L, or 1.3 g/L).

FIG. 4 is a representative graph showing the supernatant titers of ranibizumab (mg/L) over time in recombinant bacteria cultures using liquid culture media including different concentrations of MgSO₄.7H₂O (0.21 g/L, 0.32 g/L, 0.64 g/L, or 1.3 g/L).

FIG. 5 is a representative graph showing the periplasm titers of ranibizumab (mg/L) over time in recombinant bacteria cultures using liquid culture media including different concentrations of MgSO₄.

FIG. 6 is a representative graph of the concentration of ranibizumab (mg/L) in supernatant of recombinant protein production medium (plus signs) and periplasm (circles) over time.

FIG. 7 is a representative graph of a sterilization profile of temperature (oC) over time (hours) as described in Example 1.

FIG. 8 is a representative graph of a run profile as described in Example 1.

FIG. 9 is a representative graph of optical density (OD) and wet cell weight (g/g) profile as described in Example 1.

FIG. 10 is a representative graph of glucose concentration, pump rate and feed weight as described in Example 1.

FIG. 11 is a representative image of a sodium dodecyl sulfate polyacrylamide gel electrophoresis (SDS-PAGE). Lane 1: Molecular Weight Markers (M.W.), 5 μL; lane 2: T=16, 4 μL, lane 3: T=19, 4 μL; lane 4: T=22, 4 μL; lane 5: T=25, 4 μL; lane 6: T=28, 4 μL; lane 7: T=31, 4 μL; lane 8: T=34, 4 μL; lane 9: T=36, 4 μL; lane 10: T=38, 4 μL; lane 11: T=40, 4 μL; lane 12: T=25 Sup, 10 μL; lane 13: T=28 Sup, 10 μL; lane 14: T=34 Sup, 10 μL; and lane 15: T=38 Sup, 10 μL.

FIG. 12 is a representative graph of a sterilization profile of temperature (oC) over time (hours) as described in Example 2.

FIG. 13 is a representative graph of glucose concentration, pump rate and feed weight as described in Example 2.

FIG. 14 is a representative graph of a run profile as described in Example 2.

FIG. 15 is a representative graph of OD and wet cell weight (g/g) profile as described in Example 2.

FIG. 16 is a representative graph of phosphate in supernatant (nM) over time (hours).

FIG. 17 is a representative image of a sodium dodecyl sulfate polyacrylamide gel electrophoresis (SDS-PAGE). Lane 3: Molecular Weight Markers (M.W.), 5 μL; lane 4: T=10P, 4 μL, lane 5: T=16P, 4 μL; lane 6: T=18P, 4 μL; lane 7: T=24P, 4 μL; lane 8: T=30P, 8 μL; lane 9: T=10S, 8 μL; lane 10: T=16S, 8 μL; lane 11: T=18S, 8 μL; lane 12: T=24S, 8 μL; lane 13: T=30S, 8 μL.

FIG. 18 is a representative graph of a sterilization profile of temperature (oC) over time (hours) as described in Example 3.

FIG. 19 is a representative graph of glucose concentration, pump rate and feed weight as described in Example 3.

FIG. 20 is a representative graph of a run profile as described in Example 3.

FIG. 21 is a representative graph of OD and wet cell weight (g/g) profile as described in Example 3.

FIG. 22 is a representative graph of inorganic phosphate in supernatant (nM) over time (hours).

FIG. 23 is a representative image of a sodium dodecyl sulfate polyacrylamide gel electrophoresis (SDS-PAGE). Lane 3: Molecular Weight Markers (M.W.), 5 μL; lane 4: T=11P, 4 μL, lane 5: T=19P, 4 μL; lane 6: T=25P, 4 μL; lane 7: T=29P, 4 μL; lane 8: T=34.5P, 8 L; lane 9: T=11S, 8 μL; lane 10: T=19S, 8 μL; lane 11: T=25S, 8 μL; lane 12: T=29S, 8 μL; lane 13: T=34.5S, 8 μL.

FIG. 24 is a graph showing the supernatant titer of ranibizumab (mg/L) over time in a recombinant bacteria batch culture using a liquid culture medium including either a low PO₄ concentration (1.30 g/L K₂HPO₄ and 3.35 g/L KH₂PO₄) or a high PO₄ concentration (2.60 g/L K₂HPO₄ and 6.70 g/L KH₂PO₄).

FIG. 25 is a graph showing the perisplasm titer (mg/L) of ranibizumab over time in a recombinant bacteria batch culture using a liquid culture medium including either a low PO₄ concentration (1.30 g/L K₂HPO₄ and 3.35 g/L KH₂PO₄) or a high PO₄ concentration (2.60 g/L K₂HPO₄ and 6.70 g/L KH₂PO₄).

FIG. 26 is a graph showing the optical density of a recombinant bacteria batch culture producing ranibizumab using a liquid culture medium including either a low PO₄ concentration (1.30 g/L K₂HPO₄ and 3.35 g/L KH₂PO₄) or a high PO₄ concentration (2.60 g/L K₂HPO₄ and 6.70 g/L KH₂PO₄.

### DETAILED DESCRIPTION

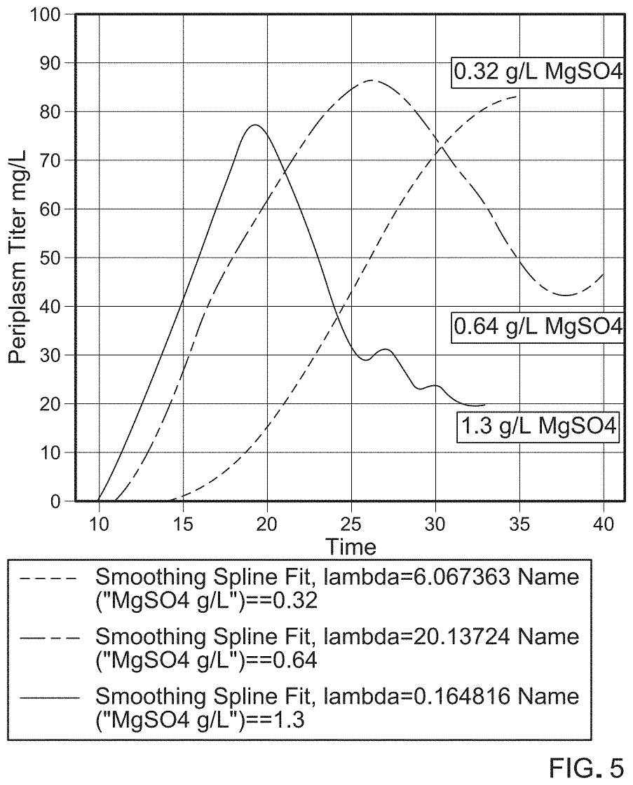

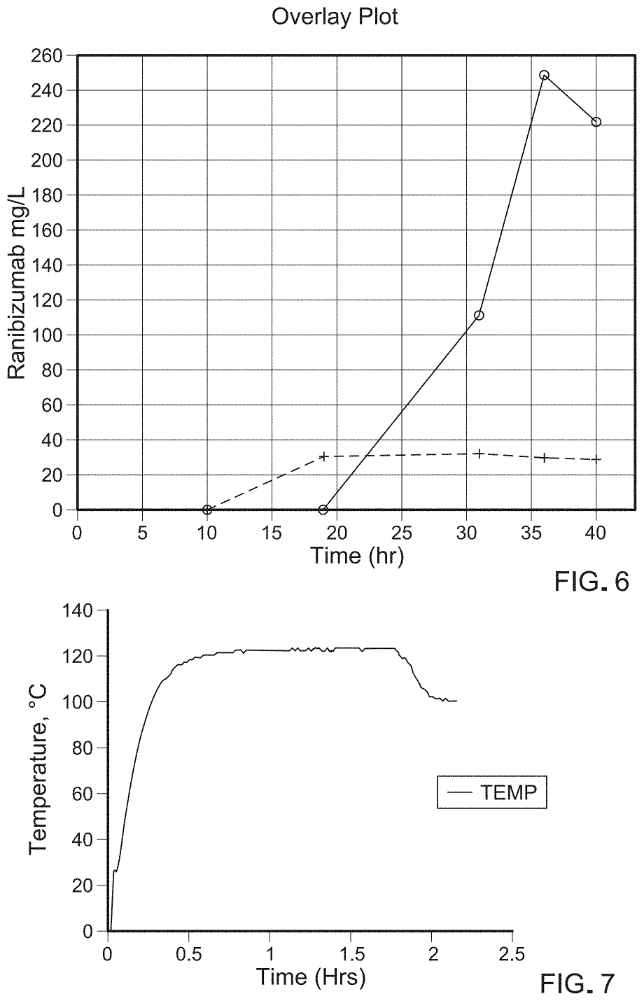

The present invention is based on the discovery that culturing a recombinant bacterium in a liquid culture medium and/or a recombinant protein production medium including about 0.3 mM to about 300 mM Mg²⁺ results in an increase in the amount of recombinant protein released from the periplasmic space of the recombinant bacterium into the liquid culture medium and/or the recombinant protein production medium (see, FIGS. 3-6). In view of this discovery, provided here are methods of producing a recombinant protein that include: providing a recombinant bacterium comprising a nucleic acid encoding a recombinant protein; and culturing the recombinant bacterium in a liquid culture medium including about 0.3 mM to about 300 mM of Mg2+ under conditions sufficient for the production and release of the recombinant protein into the culture medium.

The methods provided herein can result, e.g., in the production of a recombinant protein production medium that is significantly free of host cell proteins and/or endotoxin (e.g., where the amount of host cell protein and/or endotoxin present in the recombinant protein production medium is less than about 20% w/w, less than about 18% w/w, less than about 16% w/w, less than about 14% w/w, less than about 12% w/w, less than about 10% w/w, less than about 8% w/w, less than about 6% w/w, less than about 4% w/w, less than about 2% w/w, or less than about 1% w/w (host cell protein and/or endotoxin compared to total protein) (where the recombinant protein production medium has not been treated to remove any host cell proteins and/or endotoxin).

The methods provided herein can result, e.g., in the production of a recombinant protein production medium that is significantly free of host cell proteins and/or endotoxin (e.g., where the amount of host cell protein and/or endotoxin present in the recombinant protein production medium is about 1% to about 20% w/w, about 1% to about 18% w/w, about 1% to about 16% w/w, about 1% to about 14% w/w, about 1% to about 12% w/w, about 1% to about 10% w/w, about 1% to about 8% w/w, about 1% to about 6% w/w, about 1% to about 4% w/w, or about 1% to about 2% w/w (host cell protein and/or endotoxin compared to total protein) (where the recombinant protein production medium has not been treated to remove any host cell proteins and/or endotoxin).

The methods provided herein result, e.g., in the production of a recombinant protein production medium that includes about 100 mg/L to about 300 mg/L, about 100 mg/L to about 280 mg/L, about 100 mg/L to about 260 mg/L, about 100 mg/L to about 240 mg/L, about 100 mg/L to about 220 mg/L, about 100 mg/L to about 200 mg/L, about 100 mg/L to about 180 mg/L, about 100 mg/L to about 160 mg/L, about 100 mg/L to about 140 mg/L, about 100 mg/L to about 120 mg/L, about 120 mg/L to about 300 mg/L, about 120 mg/L to about 280 mg/L, about 120 mg/L to about 260 mg/L, about 120 mg/L to about 240 mg/L, about 120 mg/L to about 220 mg/L, about 120 mg/L to about 200 mg/L, about 120 mg/L to about 180 mg/L, about 120 mg/L to about 160 mg/L, about 120 mg/L to about 140 mg/L, about 140 mg/L to about 300 mg/L, about 140 mg/L to about 280 mg/L, about 140 mg/L to about 260 mg/L, about 140 mg/L to about 240 mg/L, about 140 mg/L to about 220 mg/L, about 140 mg/L to about 200 mg/L, about 140 mg/L to about 180 mg/L, about 140 mg/L to about 160 mg/L, about 160 mg/L to about 300 mg/L, about 160 mg/L to about 280 mg/L, about 160 mg/L to about 260 mg/L, about 160 mg/L to about 240 mg/L, about 160 mg/L to about 220 mg/L, about 160 mg/L to about 200 mg/L, about 160 mg/L to about 180 mg/L, about 180 mg/L to about 300 mg/L, about 180 mg/L to about 280 mg/L, about 180 mg/L to about 260 mg/L, about 180 mg/L to about 240 mg/L, about 180 mg/L to about 220 mg/L, about 180 mg/L to about 200 mg/L, about 200 mg/L to about 300 mg/L, about 200 mg/L to about 280 mg/L, about 200 mg/L to about 260 mg/L, about 200 mg/L to about 240 mg/L, about 200 mg/L to about 220 mg/L, about 220 mg/L to about 300 mg/L, about 220 mg/L to about 280 mg/L, about 220 mg/L to about 260 mg/L, about 220 mg/L to about 240 mg/L, about 240 mg/L to about 300 mg/L, about 240 mg/L to about 280 mg/L, about 240 mg/L to about 260 mg/L, about 260 mg/L to about 300 mg/L, about 260 mg/L to about 280 mg/L, or about 280 mg/L to about 300 mg/L recombinant protein (where the methods do not involve a step of physically and/or chemically disrupting the outer membrane of the recombinant bacterium).

Some embodiments of these methods include recovering the recombinant protein, purifying the recovered recombinant protein, and formulating the purified recombinant protein, where the method only includes a total of one chromatography step, two chromatography steps, or three chromatography steps.

Various exemplary aspects of these methods are described below and can be used in any combination in the methods provided herein without limitation. Exemplary aspects of the provided methods are described below; however, one skilled in the art will appreciate that additional steps can be added to the methods described herein and other materials can be used to perform any of the steps of the methods described herein.

### Recombinant Protein

Non-limiting examples of recombinant proteins produced by the methods provided herein include immunoglobulins (including light and heavy chain immunoglobulins), antibodies, or antibody fragments (e.g., any of the antibody fragment described herein). Non-limiting examples of recombinant proteins that can be produced by the methods described herein include ranibizumab and bevacizumab.

In some embodiments, the recombinant protein is non-glycosylated.

In some embodiments, the recombinant protein is an antibody or an antigen-binding antibody fragment.

In some embodiments, the recombinant protein can be CroFab®, DigiFab®, Digibind®, ReoPro®, and Cimzia®. In some embodiments, the recombinant protein can be, e.g., TOB5-D4, LA13-IIE3, anti-MUC1, SH363-A9, SH365-C9, filgrastim (Neupogen), pegfilgrastim (Neulasta), insulin ((e.g. insulin glargine (Lantus), insulin aspart, insulin glulisine, insulin lispro (fast-acting insulin analog), insulin detemir (long-acting insulin), isophane insulin (intermediate—acting insulin)), insulin-like growth factor 1 (Mecasermin), insulin-like growth factor I and its binding protein IGFBP-3 (Mecasermin rinfabate), denileukin diftitox, endostatin, interleukin-2 (Aldesleukin), interleukin-1 (ILl) receptor antagonist, interleukin-11, interferon alpha-2a, interferon alpha-2b, interferon alpha-1b, interferon beta-1b, interferon gamma-1a, interferon gamma-1b, tasonermin, molgramostim, nartograstim, palifermin, sargramostim, salmon calcitonin, glucagon, glucagon like peptide 1 (Liraglutide), bacterial carboxypeptidase G2 (Glucarpidase), B-type natriuretic peptide, OspA (Outer surface protein A fragment from Borrelia burgdorferi), palifermin (truncade keratinocyte growth factor), parathyroid hormone, growth hormone, pegvisomant (modified GH; Somavert), reteplase (plasminogen activator; Rapilysi), somatropin (tasonermin; Humatrope), tasonermin (cytokine), urate oxidase, teriparatide (parathyroid hormone), albumin, Hepatitis B surface antigen, Hepatitis B surface antigen and hepatitis A virus inactivated, hirudine, HPV vaccine, HPV surface antigens, platelet derived growth factor-BB, rasburicase, sargramostim, cytochromes (e.g. P450 enzymes), interferon, leptin, and brolucizumab.

In some embodiments, the recombinant protein can be an antibody or an antigen-binding antibody fragment selected from the group of: abciximab, abituzumab, abrezekimab, abrilumab, actoxumab, adalimumab, adecatumumab, atidortoxumab, aducanumab, afasevikumab, alacizumab pegol, alemtuzumab, alirocumab, amatuximab, andecaliximab, anetumab ravtansine, anifrolumab, anrukinzumab, apolizumab, aprutumab ixadotin, ascrinvacumab, aselizumab, atezolizumab, atinumab, atorolimumab, avelumab, azintuxizumab vedotin, bapineuzumab, basiliximab, bavituximab, BCD-100, belantamab mafodotin, belimumab, bemarituzumab, benralizumab, berlimatoxumab, bermekimab, bersanlimab, bertilimumab, bevacizumab, bezlotoxumab, bimagrumab, bimekizumab, birtamimab, bivatuzumab mertansine, bleselumab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, carlumab, carotuximab, cBR96, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, CR6261, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, dezamizumab, dinutuximab, diridavumab, domagrozumab, dostarlimab, drozitumab, DS-8201, duligotuzumab, dupilumab, durvalumab, dusigitumab, duvortuxizumab, ecromeximab, eculizumab, efalizumab, efungumab, eldelumab, elezanumab, elgemtumab, elotuzumab, emactuzumab, emapalumab, emibetuzumab, emicizumab, enapotamab vedotin, enavatuzumab, enfortumab vedotin, enoblituzumab, enokizumab, enoticumab, ensituximab, epratuzumab, eptinezumab, erenumab, erlizumab, etaracizumab, etigilimab, etrolizumab, evinacumab, evolocumab, exbivirumab, faricimab, farletuzumab, fasinumamb, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, fulranumab, futuximab, galcanezumab, galiximab, gancotamab, ganitumab, gantenerumab, gatipotuzumab, gedivumab, gemtuzumab ozogamicin, gevokizumab, gimsilumab, girentuximab, glembatumumab vedotin, golimumab, gomiliximab, gosuranemab, guselkumab, ianalumab, ibalizumab, IBI308, icrucumab, idarucizumab, ifabotuzumab, iladatuzumab vedotin, IMAB362, imalumab, imaprelimab, imgatuzumab, inclacumab, indatuximab ravtansine, indusatumab vedotin, inebilizumab, infliximab, intelumumab, inotuzumab ozogamicin, ipilimumab, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lendalizumab, lenvervimab, lenzilumab, lerdelimumab, leronlimab, lesolimab, letolizumab, lexatumumab, libivirumab, lifastuzumab vedotin, ligelizumab, loncastuximab tesirine, losatuxizumab vedotin, lintuzumab, lirilumab, lodelcizumab, lorvotuzumab mertansine, lucatumumab, lulizumab, lumiliximab, lumretuzumab, lupartumab amadotin, lutikizumab, mapatumumab, margetuximab, marstacimab, maslimomab, mavrilimumab, matuzumab, mepolizumab, metelimumab, milatuzumab, mirikizumab, mirvetuximab soravtansine, modotuximab, mogamulizumab, monalizumab, morolimumab, mosunetuzumab, motavizumab, namilumab, naratuximab emtansine, narnatumab, natalizumab, navicixizumab, navivumab, naxitamab, nebacumab, necitumumab, nemolizumab, NEOD001, nesvacumab, netakimab, nimotuzumab, nirsevimab, nivolumab, obiltoxaximab, obinutuzumab, ocaratuzumab, ocrelizumab, ofatumumab, olaratumab, oleclumab, olendalizumab, olokizumab, omalizumab, OMS721, onartuzumab, onartuzumab, ontuxizumab, onvatilimab, opicinumab, oportuzumab monatox, orticumab, otelixizumab, otilimab, otlertuzumab, oxelumab, ozanezumab, ozoralizumab, pagibaximab, palivizumab, pmrevlumab, panitumumab, pankomab, panobacumab, parsatuzumab, pascolizumab, pasotuxizumab, pateclizumab, patritumab, PDR001, pembrolizumab, perakizumab, pertuzumab, pexelizumab, pidilizumab, pinatuzumab vedotin, placulumab, plozalizumab, pogalizumab, polatuzumab vedotin, ponezumab, porgaviximab, prasinezumab, prezalizumab, priliximab, pritoxaximab, pritumumab, PRO 140, quilizumab, radretumab, rafivirumab, ralpancizumab, ramucirumab, ranibizumab, raxibacumab, ravagalimab, ravulizumab, refanezumab, regavirumab, relatlimab, remtolumab, reslizumab, rilotumumab, rinucumab, risankizumab, rituximab, rivabazumab pegol, robatumumab, Rmab, roledumab, romikimab, romosozumab, rontalizumab, rosmantuzumab, rovalptuzumab tesirine, rovelizumab, rozanolixizumab, ruplizumab, SA237, sacituzumab govitecan, samalizumab, samrotamab vedotin, sarilumab, satralizumab, secukinumab, selicrelumab, seribantumab, setoxaximab, setrusumab, sevirumab, sibrotuzumab, SGN-CD19A, SHP647, sifalimumab, siltuximab, simtuzumab, siplizumab, sirtratumab vedotin, sirukumab, sofituzumab vedotin, solanezumab, sonepcizumab, sontuzumab, spartalizumab, stamulumab, suptavumab, sutimlimab, suvizumab, suvratoxumab, tabalumab, tacatuzumab tetraxetan, tadocizumab, talizumab, tanezumab, tarextumab, tavolimab, tefibazumab, telisotuzumab vedotin, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragotumab, tiselizumab, tisotumab vedotin, TNX-650, tocilizumab, tomuzotuximab, toralizumab, tosatoxumab, tositumomab, tovetumab, tralokinumab, trastuzumab, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandotuzumab vedotin, vantictumab, vanucizumab, vapaliximab, varisacumab, varlilumab, vatelizumab, vedolizumab, veltuzumab, vesencumab, visilizumab, vobarilizumab, volociximab, vonterolizumab, vopratelimab, vorsetuzumab, votumumab, vunakizumab, xentuzumab, XMAB-5574, zalutumumab, zanolimumab, zatuximab, zenocutuzumab, ziralimumab, and zolbetuximab, and an antigen-binding antibody fragment of any of these antibodies.

In some embodiments, the recombinant protein is an antibody or antigen-binding antibody fragment that binds to vascular endothelial growth factor (VEGF). In some embodiments, the antigen-binding antibody fragment is ranibizumab.

Ranibizumab is a recombinant humanized IgG1 kappa isotype monoclonal antibody fragment. Ranibizumab binds to and inhibits the biologic activity of human vascular endothelial growth factor A (VEGF-A). Ranibizumab, which lacks an Fc region, has a molecular weight of approximately 48 kilodaltons. Ranibizumab binds to the receptor binding site of active forms of VEGF-A, including the biologically active, cleaved form of this molecule, VEGF 110. The binding of ranibizumab to VEGF-A prevents the interaction of VEGF-A with its receptors (VEGFR1 and VEGFR2) on the surface of endothelial cells, reducing endothelial cell proliferation, vascular leakage, and new blood vessel formation. Ranibizumab has been described in U.S. Pat. No. 6,407,213 (in addition to non-human CDRs derived from the sequence of the murine antibody, ranibizumab comprises framework substitutions in the variable domains at positions 4 and 46 in the light chain (VL) and positions 49, 69, 71, 73, 76, 78, and 94 in the heavy chain (VH)), and in U.S. Pat. No. 7,060,269 (Y0317 light chain SEQ ID NO: 1 and heavy chain SEQ ID NO: 2).

Light Chain
(SEQ ID NO: 1)
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTV
Heavy Chain
(SEQ ID NO: 2)
SGGGSGSGDFDYEKMANANKGAMTENADENALQSDAKGKLDSVATDYGAA
IDGFIGDVSGLANGNGATGDFAGSNSQMAQVGDGDNSPLMNNFRQYLPSL
PQSVECRPFVFSAGKPYEFSIDCDKINLFRGVFAFLLYVATFMYVFSTFA
NILRNKES

In some embodiments, ranibizumab includes the amino acid sequence of one or both of SEQ ID NO: 3 and 4.

Light Chain of Ranibizumab
(SEQ ID NO: 3)
DIQLTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Heavy Chain of Ranibizumab
(SEQ ID NO: 4)
EVQLVESGGGLVQPGGSLRLSCAASGYDFTHYGMETNWVRQAPGKGLEWV
GWINTYTGEPTYAADFKRRFTFSLDTSKSTAYLQMETNSLRAEDTAVYYC
AKYPYYYGTSHWYFDVWGQGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKKVEPKSCDKTHL

In some embodiments, the recombinant protein has an amino acid sequence that differs from a reference protein. For example, the reference protein is ranibizumab and the recombinant protein has a conservative amino acid substitution for one or more of the amino acids of ranibizumab.

In some embodiments, the recombinant protein includes an amino acid sequence that is at least 80% (e.g., at least 81%, at least 82%, at least 83%, at least 84%, at least 85%, at least 86%, at least 88%, at least 90%, at least 92%, at least 94%, at least 95%, at least 96%, at least 97%, at least 98%, at least 99%, at least 99.5%, or 100%) identical to SEQ ID NO: 3. In some embodiments, the recombinant protein includes an amino acid sequence that is at least 80% (e.g., at least 81%, at least 82%, at least 83%, at least 84%, at least 85%, at least 86%, at least 88%, at least 90%, at least 92%, at least 94%, at least 95%, at least 96%, at least 97%, at least 98%, at least 99%, at least 99.5%, or 100%) identical to SEQ ID NO: 4.

In some embodiments, the recombinant protein includes a sequence that differs from the amino sequence of SEQ ID NO: 3 by one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) amino acids. In some embodiments, the recombinant protein includes a sequence that differs from the amino sequence of SEQ ID NO: 4 by one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) amino acids.

For example, where a recombinant protein includes a sequence that differs from the amino acid sequence of SEQ ID NO: 3 and/or SEQ ID NO: 4 by one or more amino acids, the amino acid present in SEQ ID NO: 3 and/or SEQ ID NO: 4 can be replaced by a similar amino acid. For example, a serine can be replaced by any of glycine, alanine, serine, threonine, or proline; arginine can be replaced by asparagine, lysine, glutamine, or histidine; leucine can be replaced by phenylalanine, isoleucine, valine, or methionine; proline can be replaced with glycine, alanine, serine, or threonine; alanine can be replaced with glycine, threonine, proline, or serine; valine can be replaced with methionine, phenylalanine, isoleucine, or leucine; glycine can be replaced with alanine, threonine, proline, or serine; isoleucine can be replaced with phenylalanine, valine, leucine, or methionine; phenylalanine can be replaced with tryptophan or tyrosine; tyrosine can be replaced with tryptophan or phenylalanine; cysteine can be replaced with serine or threonine; histidine can be replaced with asparagine, lysine, glutamine, or arginine; glutamine can be replaced with glutamic acid, asparagine, or aspartic acid; asparagine can be replaced with glutamic acid, aspartic acid, or glutamine; lysine can be replaced with asparagine, glutamine, arginine, or histidine; asparatic acid can be replaced with glutamic acid, asparagine, or glutamine; glutamic acid can be replaced by asparagine, aspartic acid, or glutamine; methionine can be replaced with phenylalanine, isoleucine, valine, or leucine; and tryptophan can be replaced with phenylalanine or tyrosine.

In some examples, a precursor form of the recombinant protein can include a signal sequence. Non-limiting examples of signal sequences include:

(SEQ ID NO: 5)
MKYLLPTAAAGLLLLAAQPAMA;
(SEQ ID NO: 6)
MKKTAIAIAVALAGFATVAQA;
(SEQ ID NO: 7)
MKKNIAFLLASMFVFSIATNAYA;
(SEQ ID NO: 8)
MFKFKKKFLVGLTAAFMSISMFSATASA;
(SEQ ID NO: 9)
MKQSTAILALLPLLFTPVTKA;
(SEQ ID NO: 10)
MMKRNILAVIVPALLVAGTANA;
(SEQ ID NO: 11)
MKKSTLALVVMGIVASASVQA;
(SEQ ID NO: 12)
MKIKTGARILALSALTTMMFSASALA;
(SEQ ID NO: 13)
MKVKVLSLLVPALLVAGAANA;
(SEQ ID NO: 14)
MKATKLVLGAVILGSTLLAG;
(SEQ ID NO: 15)
MMITLRKLPLAVAVAAGVMSAQAMA:
(SEQ ID NO: 16)
MRAKLLGIVLTTPIAISSFA;
(SEQ ID NO: 17)
MSIQHFRVALIPFFAAFCLPVFA;
(SEQ ID NO: 18)
MKQSTIALALLPLLFTPVTKA;
(SEQ ID NO: 19)
MKVMRTTVATVVAATLSMSAFSVFA;
and
(SEQ ID NO: 20)
MKKSLVLKASVAVATLVPMLSFA.

In some embodiments, a secreted recombinant protein is recovered and optionally purified from the recombinant protein production medium (e.g., using any of the exemplary methods known in the art).

In some embodiments, at least about 30% (e.g., at least about 40%, at least about 50%, at least about 55%, at least about 60%, at least about 65%, at least about 70%, at least about 75%, at least about 80%, at least about 85%, at least about 90%, at least about 95%, or at least about 99%) of the secreted recombinant protein is properly folded or unfolded in the recombinant protein production medium.

In some embodiments, less than about 30% (e.g., less than about 25%, less than about 20%, less than about 15%, less than about 10%, less than about 8%, less than about 6%, less than about 4%, less than about 2%, or less than about 1%) of the secreted recombinant protein is not properly folded or unfolded in the recombinant protein production medium.

### Recombinant Bacterium

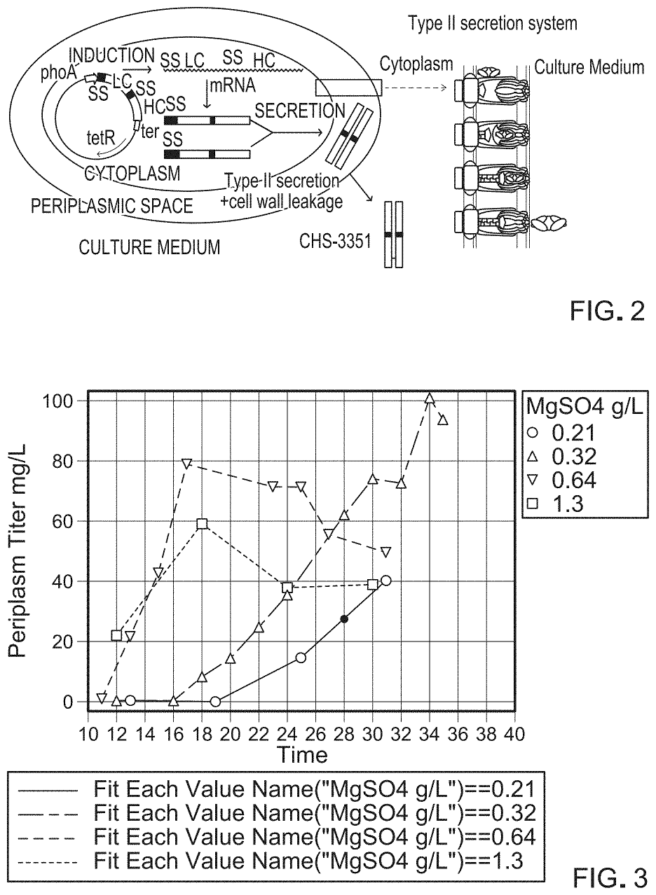

In some embodiments, the recombinant bacterium is a recombinant bacterium (e.g., a Gram-negative bacterium) including a nucleic acid encoding a recombinant protein (e.g., any of the exemplary recombinant proteins described herein or known in the art). In some embodiments of any of the recombinant bacteria described herein, the recombinant bacterium can have a type II secretion mechanism (described in, e.g., von Tils et al., Front. Cell Infect. Microbiol. 2(160):1-11, 2012, and Mergulhao et al., Biotechnol. Advances 23:177-202, 2005) (depicted in FIG. 2).

Non-limiting examples of bacterium cultured in any of the methods described herein include: an E. coli strain (e.g., a BL21 E. coli strain, a 60E4 E. coli strain, E. coli JM109 strain, E. coli MC4100 strain, E. coli DH5a strain, E. coli KS476 (degP) strain, E. coli TX1 strain, E. coli HB101 strain, E. coli JM105 strain, E. coli TOP10 strain, E. coli TOP10 F′ strain, E. coli 60E4 strain, E. coli RV308 strain, E. coli BL21 (DE3) strain, E. coli HM114 strain, E. coli TB1, E. coli K₁₂ strain, E. coli AF1000 strain, E. coli MC1061 strain, E. coli CC118 strain, E. coli RB791 strain, E. coli CC202 strain, E. coli SF110 strain, E. coli TG1 strain, E. coli HB2151 strain, E. coli XL1-Blue MRF′strain, E. coli WCM105 strain, or E. coli C600-1 strain), a Yersinia strain, an Acinetobacter strain, a Bordella strain, a Brucella strain, a Cyanobacter strain, an Enterobacter strain, a Helicobacter strain, a Klebsiella strain, a Neisseria strain, a Pasteurella strain, a Pseudomonas strain (e.g., Pseudomonas arginosa, Pseudomonas fluorescens), a Salmonella strain, Ralstonia strain (e.g., Ralstonia eutropha), and a Shigella strain.

In some embodiments, the recombinant bacterium includes at least one nucleic acid with at least one (e.g., at least two) sequence encoding a recombinant protein. For example, the recombinant bacterium can include a nucleic acid encoding an antibody light chain and an antibody heavy chain. In some examples, the recombinant bacterium can include a nucleic acid encoding a light chain variable domain and a heavy chain variable domain. In some examples, a recombinant bacterium can include a first nucleic acid encoding an antibody light chain and a second nucleic acid encoding an antibody heavy chain. In some examples, a recombinant bacterium can include a first nucleic acid encoding an light chain variable domain and a second nucleic acid encoding a heavy chain variable domain.

In some embodiments, the recombinant protein is an immunoglobulin. In still another embodiment, the immunoglobulin is light chain of an antibody, the heavy chain of antibody, or both the heavy and light chains of an antibody.

In yet another embodiment, the antigen-binding antibody fragment is ranibizumab. In another embodiment, the recombinant bacterium includes at least one nucleic acid that encodes SEQ ID NO: 3. In some examples, the recombinant bacterium includes at least one nucleic acid that encodes SEQ ID NO: 4. In some examples, the recombinant bacterium includes one nucleic acid that encodes SEQ ID NO: 3 and SEQ ID NO: 4. In some examples, the recombinant bacterium includes a first nucleic acid encoding SEQ ID NO: 3 and a second nucleic acid encoding SEQ ID NO: 4.

In some embodiments, the recombinant bacterium expresses a nucleic acid that is under control of at least one promoter (e.g., operably linked to at least one promoter). Non-limiting examples of promoters include aphoA promoter, a lac promoter, a tac promoter, aphoA promoter, a tetA promoter, a araBAD promoter, a T7 promoter, a T7/lac promoter, a lacUV5 promoter, a lacUV5 promoter, a trc promoter, or a cspA promoter. Additional promoters that can be used to express a nucleic acid in a recombinant bacterium are known in the art.

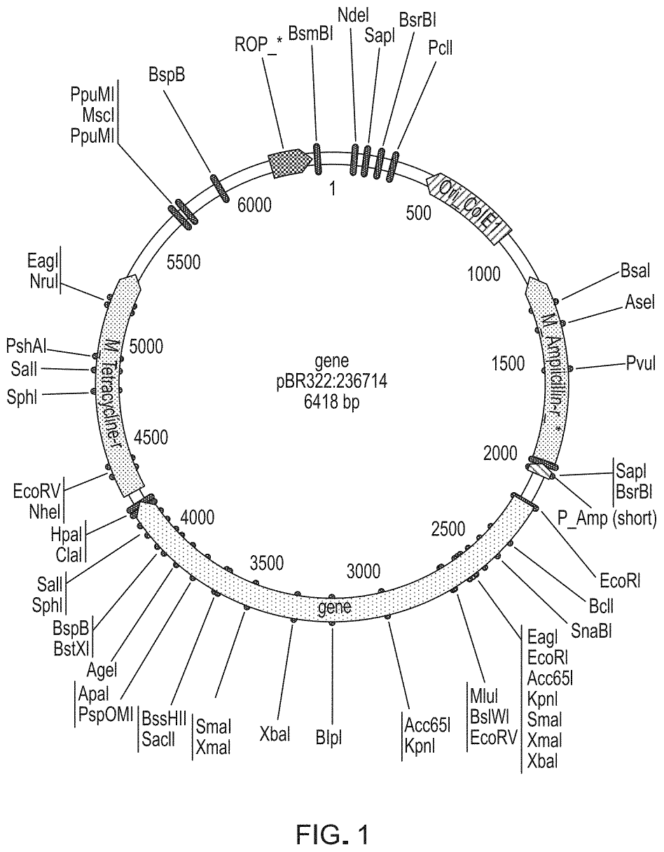

Non-limiting examples of expression vectors that can be used to express any of the recombinant proteins described herein include pOPE101-XP, pTTO-1, or pDNAbEng-1. Another example of an expression vector that can be used to express any of the recombinant proteins described herein is shown in FIG. 1. In some examples of these expression vectors, the expression can include an origin of replication. Non-limiting examples of origins of replication include pMB 1, ColE1, pUC, p15A, or pSC101.

In some examples of these expression vectors, the expression vector can further include a selectable marker, e.g., an antibiotic resistance gene (e.g., Amp, Cm, Tet, or Kan resistance gene). Additional examples of expression vectors and origins of replication and promoters that can be used in expression vectors are described in U.S. Pat. No. 5,595,898, U.S. Patent Application Publication No. 2012/0137162, U.S. Pat. No. 7,754,447, U.S. Reissued Pat. No. 44,512, European Patent No. 1664278, and U.S. Pat. No. 9,267,164 (incorporated herein by reference).

### Liquid Culture Media and Recombinant Protein Production Media

Liquid culture media and recombinant protein production media include ingredients and nutrients that are generally useful for bacterial growth (e.g., a carbon source (e.g., glucose, glutamate, galactose, citric acid, glycerol, or a combination thereof) and a nitrogen source (e.g., yeast extract, ammonium salt, nitrates)). In some embodiments, liquid culture media and/or recombinant protein production media is supplemented with an antibiotic (e.g., tetracycline), and/or a phosphate (e.g., inorganic phosphate). In some embodiments, the liquid culture media and/or the recombinant protein production media includes animal-free components. In some embodiments, the liquid culture media and/or the recombinant protein production media include tryptone and/or N—Z amine type A.

In some embodiments, the pH of the culture medium is between about 3.0 to about 8.0 (e.g., about 3.0 to about 7.5, about 3.0 to about 7.0, about 3.0 to about 6.5, about 3.0 to about 6.0, about 3.0 to about 5.5, about 3.0 to about 5.0, about 3.0 to about 4.5, about 3.0 to about 4.0, about 3.0 to about 3.5, about 4.0 to about 7.5, about 4.0 to about 7.0, about 4.0 to about 6.5, about 4.0 to about 6.0, about 4.0 to about 5.5, about 4.0 to about 5.0, about 4.0 to about 4.5, about 5.0 to about 7.5, about 5.0 to about 7.0, about 5.0 to about 6.5, about 5.0 to about 6.0, about 5.0 to about 5.5, about 6.0 to about 7.5, about 6.0 to about 7.0, about 6.0 to about 6.5, about 6.5 to about 7.5, about 6.5 to about 7.0, or about 7.0 to about 7.5).

In some embodiments, the culture medium includes about 1.0 mg/L to about 100.0 mg/L (e.g., about 1.0 mg/L to about 75 mg/L, about 10 mg/L to about 50 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 2.5 mg/L, about 2.5 mg/L to about 15.0 mg/L, about 2.5 mg/L to about 12.5 mg/L, about 2.5 mg/L to about 10.0 mg/L, about 2.5 mg/L to about 7.5 mg/L, about 2.5 mg/L to about 5.0 mg/L, about 5.0 mg/L to about 15.0 mg/L, about 5.0 mg/L to about 12.5 mg/L, about 5.0 mg/L to about 10.0 mg/L, about 5.0 mg/L to about 7.5 mg/L, about 7.5 mg/mL to about 15.0 mg/L, about 7.5 mg/L to about 12.5 mg/L, about 7.5 mg/L to about 10.0 mg/L, about 10.0 mg/L to about 15.0 mg/L, about 10.0 mg/L to about 12.5 mg/L, about 12.5 mg/L to about 15.0 mg/L, about 25 mg/L to about 75 mg/L, about 40 mg/L to about 60 mg/L, or, about 45 mg/L to about 55 mg/L) of antibiotic (e.g., carbenicillin, tetracycline). In some embodiments, the culture medium includes about 50 mg/L carbenicillin or about 10 mg/L tetracycline.

In some embodiments, the culture medium includes about 0.07 g/L to about 100 g/L, about 0.1 g/L to about 100.0 g/L, about 0.1 g/L to about 95 g/L, about 0.1 g/L to about 90 g/L, about 0.1 g/L to about 85 g/L, about 0.1 g/L to about 80 g/L, about 0.1 g/L to about 75 g/L, about 0.1 g/L to about 72 g/L, about 0.1 g/L to about 70 g/L, about 0.1 g/L to about 65 g/L, about 0.1 g/L to about 60 g/L, about 0.1 g/L to about 55 g/L, about 0.1 g/L to about 50 g/L, about 0.1 g/L to about 45 g/L, about 0.1 g/L to about 40 g/L, about 0.1 g/L to about 35 g/L, about 0.1 g/L to about 30 g/L, about 0.1 g/L to about 25 g/L, about 0.1 g/L to about 20 g/L, about 0.1 g/L to about 15 g/L, about 0.1 g/L to about 10 g/L, about 0.1 g/L to about about 0.1 g/L to about 9.5 g/L, about 0.1 g/L to about 9.0 g/L, about 0.1 g/L to about 8.5 g/L, about 0.1 g/L to about 8.0 g/L, about 0.1 g/L to about 7.5 g/L, about 0.1 g/L to about 7.0 g/L, about 0.1 g/L to about 6.5 g/L, about 0.1 g/L to about 6.0 g/L, about 0.1 g/L to about 5.5 g/L, about 0.1 g/L to about 5.0 g/L, about 0.1 g/L to about 4.5 g/L, about 0.1 g/L to about 4.0 g/L, about 0.1 g/L to about 3.5 g/L, about 0.1 g/L to about 3.0 g/L, about 0.1 g/L to about 2.5 g/L, about 0.1 g/L to about 2.0 g/L, about 0.1 g/L to about 1.5 g/L, about 0.1 g/L to about 1.0 g/L, about 0.1 g/L to about 0.5 g/L, about 0.2 g/L to about 100.0 g/L, about 0.2 g/L to about 95 g/L, about 0.2 g/L to about 90 g/L, about 0.2 g/L to about 85 g/L, about 0.2 g/L to about 80 g/L, about 0.2 g/L to about 75 g/L, about 0.2 g/L to about 72 g/L, about 0.2 g/L to about 70 g/L, about 0.2 g/L to about 65 g/L, about 0.2 g/L to about 60 g/L, about 0.2 g/L to about 55 g/L, about 0.2 g/L to about 50 g/L, about 0.2 g/L to about 45 g/L, about 0.2 g/L to about 40 g/L, about 0.2 g/L to about 35 g/L, about 0.2 g/L to about 30 g/L, about 0.2 g/L to about 25 g/L, about 0.2 g/L to about 20 g/L, about 0.2 g/L to about 15 g/L, about 0.2 g/L to about 10.0 g/L, about 0.2 g/L to about 9.5 g/L, about 0.2 g/L to about 9.0 g/L, about 0.2 g/L to about 8.5 g/L, about 0.2 g/L to about 8.0 g/L, about 0.2 g/L to about 7.5 g/L, about 0.2 g/L to about 7.0 g/L, about 0.2 g/L to about 6.5 g/L, about 0.2 g/L to about 6.0 g/L, about 0.2 g/L to about 5.5 g/L, about 0.2 g/L to about 5.0 g/L, about 0.2 g/L to about 4.5 g/L, about 0.2 g/L to about 4.0 g/L, about 0.2 g/L to about 3.5 g/L, about 0.2 g/L to about 3.0 g/L, about 0.2 g/L to about 2.5 g/L, about 0.2 g/L to about 2.0 g/L, about 0.2 g/L to about 1.5 g/L, about 0.2 g/L to about 1.0 g/L, about 0.2 g/L to about 0.5 g/L, about 0.5 g/L to about 100.0 g/L, about 0.5 g/L to about 95 g/L, about 0.5 g/L to about 90 g/L, about 0.5 g/L to about 85 g/L, about 0.5 g/L to about 80 g/L, about 0.5 g/L to about 75 g/L, about 0.5 g/L to about 72 g/L, about 0.5 g/L to about 70 g/L, about 0.5 g/L to about 65 g/L, about 0.5 g/L to about 60 g/L, about 0.5 g/L to about 55 g/L, about 0.5 g/L to about 50 g/L, about 0.5 g/L to about 45 g/L, about 0.5 g/L to about 40 g/L, about 0.5 g/L to about 35 g/L, about 0.5 g/L to about 30 g/L, about 0.5 g/L to about 25 g/L, about 0.5 g/L to about 20 g/L, about 0.5 g/L to about 15 g/L, about 0.5 g/L to about 10.0 g/L, about 0.5 g/L to about 9.5 g/L, about 0.5 g/L to about 9.0 g/L, about 0.5 g/L to about 8.5 g/L, about 0.5 g/L to about 8.0 g/L, about 0.5 g/L to about 7.5 g/L, about 0.5 g/L to about 7.0 g/L, about 0.5 g/L to about 6.5 g/L, about 0.5 g/L to about 6.0 g/L, about 0.5 g/L to about 5.5 g/L, about 0.5 g/L to about 5.0 g/L, about 0.5 g/L to about 4.5 g/L, about 0.5 g/L to about 4.0 g/L, about 0.5 g/L to about 3.5 g/L, about 0.5 g/L to about 3.0 g/L, about 0.5 g/L to about 2.5 g/L, about 0.5 g/L to about 2.0 g/L, about 0.5 g/L to about 1.5 g/L, about 0.5 g/L to about 1.0 g/L, about 1.0 g/L to about 100.0 g/L, about 1.0 g/L to about 95 g/L, about 1.0 g/L to about 90 g/L, about 1.0 g/L to about 85 g/L, about 1.0 g/L to about 80 g/L, about 1.0 g/L to about 75 g/L, about 1.0 g/L to about 72 g/L, about 1.0 g/L to about 70 g/L, about 1.0 g/L to about 65 g/L, about 1.0 g/L to about 60 g/L, about 1.0 g/L to about 55 g/L, about 1.0 g/L to about 50 g/L, about 1.0 g/L to about 45 g/L, about 1.0 g/L to about 40 g/L, about 1.0 g/L to about 35 g/L, about 1.0 g/L to about 30 g/L, about 1.0 g/L to about 25 g/L, about 1.0 g/L to about 20 g/L, about 1.0 g/L to about 15 g/L, about 1.0 g/L to about 10.0 g/L, about 1.0 g/L to about 9.5 g/L, about 1.0 g/L to about 9.0 g/L, about 1.0 g/L to about 8.5 g/L, about 1.0 g/L to about 8.0 g/L, about 1.0 g/L to about 7.5 g/L, about 1.0 g/L to about 7.0 g/L, about 1.0 g/L to about 6.5 g/L, about 1.0 g/L to about 6.0 g/L, about 1.0 g/L to about 5.5 g/L, about 1.0 g/L to about 5.0 g/L, about 1.0 g/L to about 4.5 g/L, about 1.0 g/L to about 4.0 g/L, about 1.0 g/L to about 3.5 g/L, about 1.0 g/L to about 3.0 g/L, about 1.0 g/L to about 2.5 g/L, about 1.0 g/L to about 2.0 g/L, aabout 1.0 g/L to about 1.5 g/L, about 2.0 g/L to about 100.0 g/L, about 2.0 g/L to about 95 g/L, about 2.0 g/L to about 90 g/L, about 2.0 g/L to about 85 g/L, about 2.0 g/L to about 80 g/L, about 2.0 g/L to about 75 g/L, about 2.0 g/L to about 72 g/L, about 2.0 g/L to about 70 g/L, about 2.0 g/L to about 65 g/L, about 2.0 g/L to about 60 g/L, about 2.0 g/L to about 55 g/L, about 2.0 g/L to about 50 g/L, about 2.0 g/L to about 45 g/L, about 2.0 g/L to about 40 g/L, about 2.0 g/L to about 35 g/L, about 2.0 g/L to about 30 g/L, about 2.0 g/L to about 25 g/L, about 2.0 g/L to about 20 g/L, about 2.0 g/L to about 15 g/L, about 2.0 g/L to about 10.0 g/L, about 2.0 g/L to about 9.5 g/L, about 2.0 g/L to about 9.0 g/L, about 2.0 g/L to about 8.5 g/L, about 2.0 g/L to about 8.0 g/L, about 2.0 g/L to about 7.5 g/L, about 2.0 g/L to about 7.0 g/L, about 2.0 g/L to about 6.5 g/L, about 2.0 g/L to about 6.0 g/L, about 2.0 g/L to about 5.5 g/L, about 2.0 g/L to about 5.0 g/L, about 2.0 g/L to about 4.5 g/L, about 2.0 g/L to about 4.0 g/L, about 2.0 g/L to about 3.5 g/L, about 2.0 g/L to about 3.0 g/L, about 2.0 g/L to about 2.5 g/L, about 3.0 g/L to about 100.0 g/L, about 3.0 g/L to about 95 g/L, about 3.0 g/L to about 90 g/L, about 3.0 g/L to about 85 g/L, about 3.0 g/L to about 80 g/L, about 3.0 g/L to about 75 g/L, about 3.0 g/L to about 72 g/L, about 3.0 g/L to about 70 g/L, about 3.0 g/L to about 65 g/L, about 3.0 g/L to about 60 g/L, about 3.0 g/L to about 55 g/L, about 3.0 g/L to about 50 g/L, about 3.0 g/L to about 45 g/L, about 3.0 g/L to about 40 g/L, about 3.0 g/L to about 35 g/L, about 3.0 g/L to about 30 g/L, about 3.0 g/L to about 25 g/L, about 3.0 g/L to about 20 g/L, about 3.0 g/L to about 15 g/L, about 3.0 g/L to about 10.0 g/L, about 3.0 g/L to about 9.5 g/L, about 3.0 g/L to about 9.0 g/L, about 3.0 g/L to about 8.5 g/L, about 3.0 g/L to about 8.0 g/L, about 3.0 g/L to about 7.5 g/L, about 3.0 g/L to about 7.0 g/L, about 3.0 g/L to about 6.5 g/L, about 3.0 g/L to about 6.0 g/L, about 3.0 g/L to about 5.5 g/L, about 3.0 g/L to about 5.0 g/L, about 3.0 g/L to about 4.5 g/L, about 3.0 g/L to about 4.0 g/L, about 3.0 g/L to about 3.5 g/L, about 4.0 g/L to about 100.0 g/L, about 4.0 g/L to about 95 g/L, about 4.0 g/L to about 90 g/L, about 4.0 g/L to about 85 g/L, about 4.0 g/L to about 80 g/L, about 4.0 g/L to about 75 g/L, about 4.0 g/L to about 72 g/L, about 4.0 g/L to about 70 g/L, about 4.0 g/L to about 65 g/L, about 4.0 g/L to about 60 g/L, about 4.0 g/L to about 55 g/L, about 4.0 g/L to about 50 g/L, about 4.0 g/L to about 45 g/L, about 4.0 g/L to about 40 g/L, about 4.0 g/L to about 35 g/L, about 4.0 g/L to about 30 g/L, about 4.0 g/L to about 25 g/L, about 4.0 g/L to about 20 g/L, about 4.0 g/L to about 15 g/L, about 4.0 g/L to about 10.0 g/L, about 4.0 g/L to about 9.5 g/L, about 4.0 g/L to about 9.0 g/L, about 4.0 g/L to about 8.5 g/L, about 4.0 g/L to about 8.0 g/L, about 4.0 g/L to about 7.5 g/L, about 4.0 g/L to about 7.0 g/L, about 4.0 g/L to about 6.5 g/L, about 4.0 g/L to about 6.0 g/L, about 4.0 g/L to about 5.5 g/L, about 4.0 g/L to about 5.0 g/L, about 4.0 g/L to about 4.5 g/L, about 5.0 g/L to about 100.0 g/L, about 5.0 g/L to about 95 g/L, about 5.0 g/L to about 90 g/L, about 5.0 g/L to about 85 g/L, about 5.0 g/L to about 80 g/L, about 5.0 g/L to about 75 g/L, about 5.0 g/L to about 72 g/L, about 5.0 g/L to about 70 g/L, about 5.0 g/L to about 65 g/L, about 5.0 g/L to about 60 g/L, about 5.0 g/L to about 55 g/L, about 5.0 g/L to about 50 g/L, about 5.0 g/L to about 45 g/L, about 5.0 g/L to about 40 g/L, about 5.0 g/L to about 35 g/L, about 5.0 g/L to about 30 g/L, about 5.0 g/L to about 25 g/L, about 5.0 g/L to about 20 g/L, about 5.0 g/L to about 15 g/L, about 5.0 g/L to about 10.0 g/L, about 5.0 g/L to about 9.5 g/L, about 5.0 g/L to about 9.0 g/L, about 5.0 g/L to about 8.5 g/L, about 5.0 g/L to about 8.0 g/L, about 5.0 g/L to about 7.5 g/L, about 5.0 g/L to about 7.0 g/L, about 5.0 g/L to about 6.5 g/L, about 5.0 g/L to about 6.0 g/L, about 5.0 g/L to about 5.5 g/L, about 6.0 g/L to about 100.0 g/L, about 6.0 g/L to about 95 g/L, about 6.0 g/L to about 90 g/L, about 6.0 g/L to about 85 g/L, about 6.0 g/L to about 80 g/L, about 6.0 g/L to about 75 g/L, about 6.0 g/L to about 72 g/L, about 6.0 g/L to about 70 g/L, about 6.0 g/L to about 65 g/L, about 6.0 g/L to about 60 g/L, about 6.0 g/L to about 55 g/L, about 6.0 g/L to about 50 g/L, about 6.0 g/L to about 45 g/L, about 6.0 g/L to about 40 g/L, about 6.0 g/L to about 35 g/L, about 6.0 g/L to about 30 g/L, about 6.0 g/L to about 25 g/L, about 6.0 g/L to about 20 g/L, about 6.0 g/L to about 15 g/L, about 6.0 g/L to about 10.0 g/L, about 6.0 g/L to about 9.5 g/L, about 6.0 g/L to about 9.0 g/L, about 6.0 g/L to about 8.5 g/L, about 6.0 g/L to about 8.0 g/L, about 6.0 g/L to about 7.5 g/L, about 6.0 g/L to about 7.0 g/L, about 6.0 g/L to about 6.5 g/L, about 7.0 g/L to about 100.0 g/L, about 7.0 g/L to about 95 g/L, about 7.0 g/L to about 90 g/L, about 7.0 g/L to about 85 g/L, about 7.0 g/L to about 80 g/L, about 7.0 g/L to about 75 g/L, about 7.0 g/L to about 72 g/L, about 7.0 g/L to about 70 g/L, about 7.0 g/L to about 65 g/L, about 7.0 g/L to about 60 g/L, about 7.0 g/L to about 55 g/L, about 7.0 g/L to about 50 g/L, about 7.0 g/L to about 45 g/L, about 7.0 g/L to about 40 g/L, about 7.0 g/L to about 35 g/L, about 7.0 g/L to about 30 g/L, about 7.0 g/L to about 25 g/L, about 7.0 g/L to about 20 g/L, about 7.0 g/L to about 15 g/L, about 7.0 g/L to about 10.0 g/L, about 7.0 g/L to about 9.5 g/L, about 7.0 g/L to about 9.0 g/L, about 7.0 g/L to about 8.5 g/L, about 7.0 g/L to about 8.0 g/L, a about 7.0 g/L to about 7.5 g/L, about 8.0 g/L to about 100.0 g/L, about 8.0 g/L to about 95 g/L, about 8.0 g/L to about 90 g/L, about 8.0 g/L to about 85 g/L, about 8.0 g/L to about 80 g/L, about 8.0 g/L to about 75 g/L, about 8.0 g/L to about 72 g/L, about 8.0 g/L to about 70 g/L, about 8.0 g/L to about 65 g/L, about 8.0 g/L to about 60 g/L, about 8.0 g/L to about 55 g/L, about 8.0 g/L to about 50 g/L, about 8.0 g/L to about 45 g/L, about 8.0 g/L to about 40 g/L, about 8.0 g/L to about 35 g/L, about 8.0 g/L to about 30 g/L, about 8.0 g/L to about 25 g/L, about 8.0 g/L to about 20 g/L, about 8.0 g/L to about 15 g/L, about 8.0 g/L to about 10.0 g/L, about 8.0 g/L to about 9.5 g/L, about 8.0 g/L to about 9.0 g/L, about 8.0 g/L to about 8.5 g/L, about 9.0 g/L to about 100.0 g/L, about 9.0 g/L to about 95 g/L, about 9.0 g/L to about 90 g/L, about 9.0 g/L to about 85 g/L, about 9.0 g/L to about 80 g/L, about 9.0 g/L to about 75 g/L, about 9.0 g/L to about 72 g/L, about 9.0 g/L to about 70 g/L, about 9.0 g/L to about 65 g/L, about 9.0 g/L to about 60 g/L, about 9.0 g/L to about 55 g/L, about 9.0 g/L to about 50 g/L, about 9.0 g/L to about 45 g/L, about 9.0 g/L to about 40 g/L, about 9.0 g/L to about 35 g/L, about 9.0 g/L to about 30 g/L, about 9.0 g/L to about 25 g/L, about 9.0 g/L to about 20 g/L, about 9.0 g/L to about 15 g/L, about 9.0 g/L to about 10.0 g/L, about 9.0 g/L to about 9.5 g/L, about 9.5 g/L to about 10.0 g/L, about 10.0 g/L to about 100.0 g/L, about 10.0 g/L to about 95 g/L, about 10.0 g/L to about 90 g/L, about 10.0 g/L to about 85 g/L, about 10.0 g/L to about 80 g/L, about 10.0 g/L to about 75 g/L, about 10.0 g/L to about 72 g/L, about 10.0 g/L to about 70 g/L, about 10.0 g/L to about 65 g/L, about 10.0 g/L to about 60 g/L, about 10.0 g/L to about 55 g/L, about 10.0 g/L to about 50 g/L, about 10.0 g/L to about 45 g/L, about 10.0 g/L to about 40 g/L, about 10.0 g/L to about 35 g/L, about 10.0 g/L to about 30 g/L, about 10.0 g/L to about 25 g/L, about 10.0 g/L to about 20 g/L, about 10.0 g/L to about 15 g/L, about 15 g/L to about 100.0 g/L, about 15 g/L to about 95 g/L, about 15 g/L to about 90 g/L, about 15 g/L to about 85 g/L, about 15 g/L to about 80 g/L, about 15 g/L to about 75 g/L, about 15 g/L to about 72 g/L, about 15 g/L to about 70 g/L, about 15 g/L to about 65 g/L, about 15 g/L to about 60 g/L, about 15 g/L to about 55 g/L, about 15 g/L to about 50 g/L, about 15 g/L to about 45 g/L, about 15 g/L to about 40 g/L, about 15 g/L to about 35 g/L, about 15 g/L to about 30 g/L, about 15 g/L to about 25 g/L, about 15 g/L to about 20 g/L, about 20 g/L to about 100.0 g/L, about 20 g/L to about 95 g/L, about 20 g/L to about 90 g/L, about 20 g/L to about 85 g/L, about 20 g/L to about 80 g/L, about 20 g/L to about 75 g/L, about 20 g/L to about 72 g/L, about 20 g/L to about 70 g/L, about 20 g/L to about 65 g/L, about 20 g/L to about 60 g/L, about 20 g/L to about 55 g/L, about 20 g/L to about 50 g/L, about 20 g/L to about 45 g/L, about 20 g/L to about 40 g/L, about 20 g/L to about 35 g/L, about 20 g/L to about 30 g/L, about 20 g/L to about 25 g/L, about 25 g/L to about 100.0 g/L, about 25 g/L to about 95 g/L, about 25 g/L to about 90 g/L, about 25 g/L to about 85 g/L, about 25 g/L to about 80 g/L, about 25 g/L to about 75 g/L, about 25 g/L to about 72 g/L, about 25 g/L to about 70 g/L, about 25 g/L to about 65 g/L, about 25 g/L to about 60 g/L, about 25 g/L to about 55 g/L, about 25 g/L to about 50 g/L, about 25 g/L to about 45 g/L, about 25 g/L to about 40 g/L, about 25 g/L to about 35 g/L, about 25 g/L to about 30 g/L, about 30 g/L to about 100.0 g/L, about 30 g/L to about 95 g/L, about 30 g/L to about 90 g/L, about 30 g/L to about 85 g/L, about 30 g/L to about 80 g/L, about 30 g/L to about 75 g/L, about 30 g/L to about 72 g/L, about 30 g/L to about 70 g/L, about 30 g/L to about 65 g/L, about 30 g/L to about 60 g/L, about 30 g/L to about 55 g/L, about 30 g/L to about 50 g/L, about 30 g/L to about 45 g/L, about 30 g/L to about 40 g/L, about 30 g/L to about 35 g/L, about 35 g/L to about 100.0 g/L, about 35 g/L to about 95 g/L, about 35 g/L to about 90 g/L, about 35 g/L to about 85 g/L, about 35 g/L to about 80 g/L, about 35 g/L to about 75 g/L, about 35 g/L to about 72 g/L, about 35 g/L to about 70 g/L, about 35 g/L to about 65 g/L, about 35 g/L to about 60 g/L, about 35 g/L to about 55 g/L, about 35 g/L to about 50 g/L, about 35 g/L to about 45 g/L, about 35 g/L to about 40 g/L, about 40 g/L to about 100.0 g/L, about 40 g/L to about 95 g/L, about 40 g/L to about 90 g/L, about 40 g/L to about 85 g/L, about 40 g/L to about 80 g/L, about 40 g/L to about 75 g/L, about 40 g/L to about 72 g/L, about 40 g/L to about 70 g/L, about 40 g/L to about 65 g/L, about 40 g/L to about 60 g/L, about 40 g/L to about 55 g/L, about 40 g/L to about 50 g/L, about 40 g/L to about 45 g/L, about 45 g/L to about 100.0 g/L, about 45 g/L to about 95 g/L, about 45 g/L to about 90 g/L, about 45 g/L to about 85 g/L, about 45 g/L to about 80 g/L, about 45 g/L to about 75 g/L, about 45 g/L to about 72 g/L, about 45 g/L to about 70 g/L, about 45 g/L to about 65 g/L, about 45 g/L to about 60 g/L, about 45 g/L to about 55 g/L, about 45 g/L to about 50 g/L, about 50 g/L to about 100.0 g/L, about 50 g/L to about 95 g/L, about 50 g/L to about 90 g/L, about 50 g/L to about 85 g/L, about 50 g/L to about 80 g/L, about 50 g/L to about 75 g/L, about 50 g/L to about 72 g/L, about 50 g/L to about 70 g/L, about 50 g/L to about 65 g/L, about 50 g/L to about 60 g/L, about 50 g/L to about 55 g/L, about 55 g/L to about 100.0 g/L, about 55 g/L to about 95 g/L, about 55 g/L to about 90 g/L, about 55 g/L to about 85 g/L, about 55 g/L to about 80 g/L, about 55 g/L to about 75 g/L, about 55 g/L to about 72 g/L, about 55 g/L to about 70 g/L, about 55 g/L to about 65 g/L, about 55 g/L to about 60 g/L, about 60 g/L to about 100.0 g/L, about 60 g/L to about 95 g/L, about 60 g/L to about 90 g/L, about 60 g/L to about 85 g/L, about 60 g/L to about 80 g/L, about 60 g/L to about 75 g/L, about 60 g/L to about 72 g/L, about 60 g/L to about 70 g/L, about 60 g/L to about 65 g/L, about 65 g/L to about 100.0 g/L, about 65 g/L to about 95 g/L, about 65 g/L to about 90 g/L, about 65 g/L to about 85 g/L, about 65 g/L to about 80 g/L, about 65 g/L to about 75 g/L, about 65 g/L to about 72 g/L, about 65 g/L to about 70 g/L, about 70 g/L to about 100.0 g/L, about 70 g/L to about 95 g/L, about 70 g/L to about 90 g/L, about 70 g/L to about 85 g/L, about 70 g/L to about 80 g/L, about 70 g/L to about 75 g/L, about 75 g/L to about 100.0 g/L, about 75 g/L to about 95 g/L, about 75 g/L to about 90 g/L, about 75 g/L to about 85 g/L, about 75 g/L to about 80 g/L, about 80 g/L to about 100.0 g/L, about 80 g/L to about 95 g/L, about 80 g/L to about 90 g/L, about 80 g/L to about 85 g/L, about 85 g/L to about 100.0 g/L, about 85 g/L to about 95 g/L, about 85 g/L to about 90 g/L, about 90 g/L to about 100.0 g/L, about 90 g/L to about 95 g/L, or about 95 g/L to about 100 g/L of a magnesium salt (e.g., MgSO₄.7H₂O or any of the other magnesium salts described herein or known in the art).

In some embodiments, the liquid culture medium and/or the recombinant protein culture medium includes about 0.1 mM to about 300 mM, about 0.1 mM to about 275 mM, about 0.1 mM to about 250 mM, about 0.1 mM to about 225 mM, about 0.1 mM to about 200 mM, about 0.1 mM to about 175 mM, about 0.1 mM to about 150 mM, about 0.1 mM to about 125 mM, about 0.1 mM to about 100 mM, about 0.1 mM to about 75 mM, about 0.1 mM to about 50 mM, about 0.1 mM to about 41 mM, about 0.1 mM to about 40 mM, about 0.1 mM to about 35 mM, about 0.1 mM to about 30 mM, about 0.1 mM to about 25 mM, about 0.1 mM to about 20 mM, about 0.1 mM to about 10 mM, about 0.1 mM to about 5 mM, about 0.1 mM to about 2 mM, about 0.1 mM to about 1.0 mM, about 0.1 mM to about 0.5 mM, about 0.2 mM to about 300 mM, about 0.2 mM to about 275 mM, about 0.2 mM to about 250 mM, about 0.2 mM to about 225 mM, about 0.2 mM to about 200 mM, about 0.2 mM to about 175 mM, about 0.2 mM to about 150 mM, about 0.2 mM to about 125 mM, about 0.2 mM to about 100 mM, about 0.2 mM to about 75 mM, about 0.2 mM to about 50 mM, about 0.2 mM to about 41 mM, about 0.2 mM to about 40 mM, about 0.2 mM to about 35 mM, about 0.2 mM to about 30 mM, about 0.2 mM to about 25 mM, about 0.2 mM to about 20 mM, about 0.2 mM to about 10 mM, about 0.2 mM to about 5 mM, about 0.2 mM to about 2 mM, about 0.2 mM to about 1.0 mM, about 0.2 mM to about 0.5 mM, about 0.5 mM to about 300 mM, about 0.5 mM to about 275 mM, about 0.5 mM to about 250 mM, about 0.5 mM to about 225 mM, about 0.5 mM to about 200 mM, about 0.5 mM to about 175 mM, about 0.5 mM to about 150 mM, about 0.5 mM to about 125 mM, about 0.5 mM to about 100 mM, about 0.5 mM to about 75 mM, about 0.5 mM to about 50 mM, about 0.5 mM to about 41 mM, about 0.5 mM to about 40 mM, about 0.5 mM to about 35 mM, about 0.5 mM to about 30 mM, about 0.5 mM to about 25 mM, about 0.5 mM to about 20 mM, about 0.5 mM to about 10 mM, about 0.5 mM to about 5 mM, about 0.5 mM to about 2 mM, about 0.5 mM to about 1.0 mM, about 1.0 mM to about 300 mM, about 1.0 mM to about 275 mM, about 1.0 mM to about 250 mM, about 1.0 mM to about 225 mM, about 1.0 mM to about 200 mM, about 1.0 mM to about 175 mM, about 1.0 mM to about 150 mM, about 1.0 mM to about 125 mM, about 1.0 mM to about 100 mM, about 1.0 mM to about 75 mM, about 1.0 mM to about 50 mM, about 1.0 mM to about 41 mM, about 1.0 mM to about 40 mM, about 1.0 mM to about 35 mM, about 1.0 mM to about 30 mM, about 1.0 mM to about 25 mM, about 1.0 mM to about 20 mM, about 1.0 mM to about 10 mM, about 1.0 mM to about 5 mM, about 1.0 mM to about 2 mM, about 2 mM to about 41 mM, about 2 mM to about 300 mM, about 2 mM to about 275 mM, about 2 mM to about 250 mM, about 2 mM to about 225 mM, about 2 mM to about 200 mM, about 2 mM to about 175 mM, about 2 mM to about 150 mM, about 2 mM to about 125 mM, about 2 mM to about 100 mM, about 2 mM to about 75 mM, about 2 mM to about 50 mM, about 2 mM to about 41 mM, about 2 mM to about 40 mM, about 2 mM to about 35 mM, about 2 mM to about 30 mM, about 2 mM to about 25 mM, about 2 mM to about 20 mM, about 2 mM to about 10 mM, about 2 mM to about 5 mM, about 5 mM to about 300 mM, about 5 mM to about 275 mM, about 5 mM to about 250 mM, about 5 mM to about 225 mM, about 5 mM to about 200 mM, about 5 mM to about 175 mM, about 5 mM to about 150 mM, about 5 mM to about 125 mM, about 5 mM to about 100 mM, about 5 mM to about 75 mM, about 5 mM to about 50 mM, about 5 mM to about 41 mM, about 5 mM to about 40 mM, about 5 mM to about 35 mM, about 5 mM to about 30 mM, about 5 mM to about 25 mM, about 5 mM to about 20 mM, about 5 mM to about 10 mM, about 10 mM to about 300 mM, about 10 mM to about 275 mM, about 10 mM to about 250 mM, about 10 mM to about 225 mM, about 10 mM to about 200 mM, about 10 mM to about 175 mM, about 10 mM to about 150 mM, about 10 mM to about 125 mM, about 10 mM to about 100 mM, about 10 mM to about 75 mM, about 10 mM to about 50 mM, about 10 mM to about 41 mM, about 10 mM to about 40 mM, about 10 mM to about 35 mM, about 10 mM to about 30 mM, about 10 mM to about 25 mM, about 10 mM to about 20 mM, about 20 mM to about 300 mM, about 20 mM to about 275 mM, about 20 mM to about 250 mM, about 20 mM to about 225 mM, about 20 mM to about 200 mM, about 20 mM to about 175 mM, about 20 mM to about 150 mM, about 20 mM to about 125 mM, about 20 mM to about 100 mM, about 20 mM to about 75 mM, about 20 mM to about 50 mM, about 20 mM to about 41 mM, about 20 mM to about 40 mM, about 20 mM to about 35 mM, about 20 mM to about 30 mM, about 20 mM to about 25 mM, about 25 mM to about 300 mM, about 25 mM to about 275 mM, about 25 mM to about 250 mM, about 25 mM to about 225 mM, about 25 mM to about 200 mM, about 25 mM to about 175 mM, about 25 mM to about 150 mM, about 25 mM to about 125 mM, about 25 mM to about 100 mM, about 25 mM to about 75 mM, about 25 mM to about 50 mM, about 25 mM to about 41 mM, about 25 mM to about 40 mM, about 25 mM to about 35 mM, about 25 mM to about 30 mM, about 30 mM to about 300 mM, about 30 mM to about 275 mM, about 30 mM to about 250 mM, about 30 mM to about 225 mM, about 30 mM to about 200 mM, about 30 mM to about 175 mM, about 30 mM to about 150 mM, about 30 mM to about 125 mM, about 30 mM to about 100 mM, about 30 mM to about 75 mM, about 30 mM to about 50 mM, about 30 mM to about 41 mM, about 30 mM to about 40 mM, about 30 mM to about 35 mM, about 35 mM to about 300 mM, about 35 mM to about 275 mM, about 35 mM to about 250 mM, about 35 mM to about 225 mM, about 35 mM to about 200 mM, about 35 mM to about 175 mM, about 35 mM to about 150 mM, about 35 mM to about 125 mM, about 35 mM to about 100 mM, about 35 mM to about 75 mM, about 35 mM to about 50 mM, about 35 mM to about 41 mM, about 35 mM to about 40 mM, about 40 mM to about 300 mM, about 40 mM to about 275 mM, about 40 mM to about 250 mM, about 40 mM to about 225 mM, about 40 mM to about 200 mM, about 40 mM to about 175 mM, about 40 mM to about 150 mM, about 40 mM to about 125 mM, about 40 mM to about 100 mM, about 40 mM to about 75 mM, about 40 mM to about 50 mM, about 50 mM to about 300 mM, about 50 mM to about 275 mM, about 50 mM to about 250 mM, about 50 mM to about 225 mM, about 50 mM to about 200 mM, about 50 mM to about 175 mM, about 50 mM to about 150 mM, about 50 mM to about 125 mM, about 50 mM to about 100 mM, about 50 mM to about 75 mM, about 75 mM to about 300 mM, about 75 mM to about 275 mM, about 75 mM to about 250 mM, about 75 mM to about 225 mM, about 75 mM to about 200 mM, about 75 mM to about 175 mM, about 75 mM to about 150 mM, about 75 mM to about 125 mM, about 75 mM to about 100 mM, about 100 mM to about 300 mM, about 100 mM to about 275 mM, about 100 mM to about 250 mM, about 100 mM to about 225 mM, about 100 mM to about 200 mM, about 100 mM to about 175 mM, about 100 mM to about 150 mM, about 100 mM to about 125 mM, about 125 mM to about 300 mM, about 125 mM to about 275 mM, about 125 mM to about 250 mM, about 125 mM to about 225 mM, about 125 mM to about 200 mM, about 125 mM to about 175 mM, about 125 mM to about 150 mM, about 150 mM to about 300 mM, about 150 mM to about 275 mM, about 150 mM to about 250 mM, about 150 mM to about 225 mM, about 150 mM to about 200 mM, about 150 mM to about 175 mM, about 175 mM to about 300 mM, about 175 mM to about 275 mM, about 175 mM to about 250 mM, about 175 mM to about 225 mM, about 175 mM to about 200 mM, about 200 mM to about 300 mM, about 200 mM to about 275 mM, about 200 mM to about 250 mM, about 200 mM to about 225 mM, about 225 mM to about 300 mM, about 225 mM to about 275 mM, about 225 mM to about 250 mM, about 250 mM to about 300 mM, about 250 mM to about 275 mM, or about 275 mM to about 300 mM of a magnesium ion (e.g., Mg²⁺).

In some embodiments, the liquid culture media and/or the recombinant protein production medium includes about 1 g/L to about 25 g/L of phosphate (e.g., inorganic phosphate). In other embodiments, the culture media includes about 2 g/L to about 15 g/L, about 3 g/L to about 8 g/L, about 5 g/L to about 20 g/L, about 5 g/L to about 15 g/L, about 5 g/L to about 10 g/L, about 10 g/L to about 25 g/L, about 10 g/L to about 20 g/L, about 10 g/L to about 15 g/L, about 15 g/L to about 25 g/L, or about 15 g/L to about 20 g/L of phosphate (e.g., inorganic phosphate).

In some embodiments, the liquid culture media and/or the recombinant protein production medium includes about 1 g/L to about 25 g/L of a phosphate salt. In other embodiments, the culture media includes about 2 g/L to about 15 g/L, about 4 g/L to about 12 g/L, about 4 g/L to about 10 g/L, about 4 g/L to about 9 g/L, about 5 g/L to about 20 g/L, about 5 g/L to about 15 g/L, about 5 g/L to about 10 g/L, about 10 g/L to about 25 g/L, about 10 g/L to about 20 g/L, about 10 g/L to about 15 g/L, about 15 g/L to about 25 g/L, or about 15 g/L to about 20 g/L of phosphate (e.g., K₂HPO₄, KH₂PO₄).

In some examples, the volume of the liquid culture medium at the start of the culturing is about 50 mL to about 1000 L, about 50 mL to about 750 L, about 50 mL to about 550 L, about 50 mL to about 500 L, about 50 mL to about 250 L, about 50 mL to about 200 L, about 50 mL to about 100 L, about 50 mL to about 90 L, about 50 mL to about 80 L, about 50 mL to about 70 L, about 50 mL to about 60 L, about 50 mL to about 50 L, about 50 mL to about 40 L, about 50 mL to about 30 L, about 50 mL to about 20 L, about 50 mL to about 10 L, about 50 mL to about 5 L, about 50 mL to about 1 L, about 50 mL to about 750 mL, about 50 mL to about 500 mL, about 50 mL to about 250 mL, about 50 mL to about 100 mL, about 100 mL to about 100 L, about 100 mL to about 90 L, about 100 mL to about 80 L, about 100 mL to about 70 L, about 100 mL to about 60 L, about 100 mL to about 50 L, about 100 mL to about 40 L, about 100 mL to about 30 L, about 100 mL to about 20 L, about 100 mL to about 10 L, about 100 mL to about 5 L, about 100 mL to about 1 L, about 100 mL to about 750 mL, about 100 mL to about 500 mL, about 100 mL to about 250 mL, about 250 mL to about 100 L, about 250 mL to about 90 L, about 250 mL to about 80 L, about 250 mL to about 70 L, about 250 mL to about 60 L, about 250 mL to about 50 L, about 250 mL to about 40 L, about 250 mL to about 30 L, about 250 mL to about 20 L, about 250 mL to about 10 L, about 250 mL to about 5 L, about 250 mL to about 1 L, about 250 mL to about 750 mL, about 250 mL to about 500 mL, about 500 mL to about 100 L, about 500 mL to about 90 L, about 500 mL to about 80 L, about 500 mL to about 70 L, about 500 mL to about 60 L, about 500 mL to about 50 L, about 500 mL to about 40 L, about 500 mL to about 30 L, about 500 mL to about 20 L, about 500 mL to about 10 L, about 500 mL to about 5 L, about 500 mL to about 1 L, about 500 mL to about 750 mL, about 750 mL to about 100 L, about 750 mL to about 90 L, about 750 mL to about 80 L, about 750 mL to about 70 L, about 750 mL to about 60 L, about 750 mL to about 50 L, about 750 mL to about 40 L, about 750 mL to about 30 L, about 750 mL to about 20 L, about 750 mL to about 10 L, about 750 mL to about 5 L, about 750 mL to about 1 L, about 1 L to about 100 L, about 1 L to about 90 L, about 1 L to about 80 L, about 1 L to about 70 L, about 1 L to about 60 L, about 1 L to about 50 L, about 1 L to about 40 L, about 1 L to about 30 L, about 1 L to about 20 L, about 1 L to about 10 L, about 1 L to about 5 L, about 5 L to about 100 L, about 5 L to about 90 L, about 5 L to about 80 L, about 5 L to about 70 L, about 5 L to about 60 L, about 5 L to about 50 L, about 5 L to about 40 L, about 5 L to about 30 L, about 5 L to about 20 L, about 5 L to about 10 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 100 L, about 40 L to about 90 L, about 40 L to about 80 L, about 40 L to about 70 L, about 40 L to about 60 L, about 40 L to about 50 L, about 50 L to about 100 L, about 50 L to about 90 L, about 50 L to about 80 L, about 50 L to about 70 L, about 50 L to about 60 L, about 60 L to about 100 L, about 60 L to about 90 L, about 60 L to about 80 L, about 60 L to about 70 L, about 70 L to about 100 L, about 70 L to about 90 L, about 70 L to about 80 L, about 80 L to about 100 L, about 80 L to about 90 L, or about 90 L to about 100 L).

### Cell Culturing

Cell culturing includes incubating a recombinant bacteria (e.g., any of the recombinant bacteria described herein) in a liquid culture medium under a controlled set of conditions. In some embodiments, cell culturing includes a proliferation phase (i.e., cell mass accumulation and rapid cell growth) and a production phase (i.e., recombinant protein production phase). In some aspects, the production phase is initiated upon the depletion of a component of the cell culture medium (e.g., depletion of phosphate).

In some embodiments, the proliferation phase and the production phase occur sequentially. In some embodiments, the proliferation phase and the production phase occur in the same vessel (e.g., at substantially the same time). In other embodiments, the proliferation phase and the production phase occur in different vessels. In some embodiments, more than one proliferation phase or production phase can occur in a production run. For example, a first proliferation phase may take place in small vessel (e.g., a shake flask) with the resulting cell culture transferred to a larger vessel (e.g., a bioreactor or fermentor), where another proliferation phase takes place before a production phase begins in the same larger vessel.

In some embodiments, the proliferation phase results in a culture having an optical density (OD) at 600 nm of about 1 to about 200, about 1 to about 180, about 1 to about 160, about 1 to about 140, about 1 to about 120, about 1 to about 100, about 1 to about 80, about 1 to about 60, about 1 to about 40, about 1 to about 20, about 1 to about 10, about 10 to about 200, about 10 to about 180, about 10 to about 160, about 10 to about 140, about 10 to about 120, about 10 to about 100, about 10 to about 80, about 10 to about 60, about 10 to about 40, about 10 to about 20, about 20 to about 200, about 20 to about 180, about 20 to about 160, about 20 to about 140, about 20 to about 120, about 20 to about 100, about 20 to about 80, about 20 to about 60, about 20 to about 40, about 40 to about 200, about 40 to about 180, about 40 to about 160, about 40 to about 140, about 40 to about 120, about 40 to about 100, about 40 to about 80, about 40 to about 60, about 60 to about 200, about 60 to about 180, about 60 to about 160, about 60 to about 140, about 60 to about 120, about 60 to about 100, about 60 to about 80, about 80 to about 200, about 80 to about 180, about 80 to about 160, about 80 to about 140, about 80 to about 120, about 80 to about 100, about 100 to about 200, about 100 to about 180, about 100 to about 160, about 100 to about 140, about 100 to about 120, about 120 to about 200, about 120 to about 180, about 120 to about 160, about 120 to about 140, about 140 to about 200, about 140 to about 180, about 140 to about 160, about 160 to about 200, about 160 to about 180, or about 180 to about 200.

In some embodiments, the proliferation phase results in a culture that includes about 10⁶ to about 10¹⁰ colony forming units (CFUs)/mL.

In some embodiments, the production phase results in a recombinant protein production medium that includes about 100 mg/L to about 300 mg/L, about 100 mg/L to about 280 mg/L, about 100 mg/L to about 260 mg/L, about 100 mg/L to about 240 mg/L, about 100 mg/L to about 220 mg/L, about 100 mg/L to about 200 mg/L, about 100 mg/L to about 180 mg/L, about 100 mg/L to about 160 mg/L, about 100 mg/L to about 140 mg/L, about 100 mg/L to about 120 mg/L, about 120 mg/L to about 300 mg/L, about 120 mg/L to about 280 mg/L, about 120 mg/L to about 260 mg/L, about 120 mg/L to about 240 mg/L, about 120 mg/L to about 220 mg/L, about 120 mg/L to about 200 mg/L, about 120 mg/L to about 180 mg/L, about 120 mg/L to about 160 mg/L, about 120 mg/L to about 140 mg/L, about 140 mg/L to about 300 mg/L, about 140 mg/L to about 280 mg/L, about 140 mg/L to about 260 mg/L, about 140 mg/L to about 240 mg/L, about 140 mg/L to about 220 mg/L, about 140 mg/L to about 200 mg/L, about 140 mg/L to about 180 mg/L, about 140 mg/L to about 160 mg/L, about 160 mg/L to about 300 mg/L, about 160 mg/L to about 280 mg/L, about 160 mg/L to about 260 mg/L, about 160 mg/L to about 240 mg/L, about 160 mg/L to about 220 mg/L, about 160 mg/L to about 200 mg/L, about 160 mg/L to about 180 mg/L, about 180 mg/L to about 300 mg/L, about 180 mg/L to about 280 mg/L, about 180 mg/L to about 260 mg/L, about 180 mg/L to about 240 mg/L, about 180 mg/L to about 220 mg/L, about 180 mg/L to about 200 mg/L, about 200 mg/L to about 300 mg/L, about 200 mg/L to about 280 mg/L, about 200 mg/L to about 260 mg/L, about 200 mg/L to about 240 mg/L, about 200 mg/L to about 220 mg/L, about 220 mg/L to about 300 mg/L, about 220 mg/L to about 280 mg/L, about 220 mg/L to about 260 mg/L, about 220 mg/L to about 240 mg/L, about 240 mg/L to about 300 mg/L, about 240 mg/L to about 280 mg/L, about 240 mg/L to about 260 mg/L, about 260 mg/L to about 300 mg/L, about 260 mg/L to about 280 mg/L, or about 280 mg/L to about 300 mg/L recombinant protein

In some embodiments, the culturing is fed batch culturing. In some embodiments, the fed batch culturing includes continuously or periodically adding a feed liquid culture medium to the recombinant protein production medium. In some embodiments, the fed batch culturing includes continuously adding to the recombinant protein production medium a volume of a feed liquid culture medium. In some embodiments, the fed batch culturing includes periodically adding to the recombinant protein production medium a volume of a feed liquid culture medium. In a non-limiting example, periodically adding a feed liquid culture medium to the recombinant protein production medium includes adding a volume of a feed liquid culture medium once every 6, 12, or 24 hours.

In some embodiments, the culturing is performed using a shake flask. In some embodiments, the culturing is performed using a bioreactor or fermentor. In some embodiments, the culturing is performed using a fed batch bioreactor or fermentor.

In some embodiments, the volume of the recombinant protein production medium at the end of the culturing is about 50 mL to about 500 L, about 50 mL to about 450 L, about 50 mL to about 400 L, about 50 mL to about 350 L, about 50 mL to about 300 L, about 50 mL to about 250 L, about 50 mL to about 200 L, about 50 mL to about 150 L, about 50 mL to about 100 L, about 50 mL to about 50 L, about 50 mL to about 10 L, about 50 mL to about 5 L, about 50 mL to about 1 L, about 50 mL to about 500 mL, about 50 mL to about 250 mL, about 250 mL to about 500 L, about 250 mL to about 450 L, about 250 mL to about 400 L, about 250 mL to about 350 L, about 250 mL to about 300 L, about 250 mL to about 250 L, about 250 mL to about 200 L, about 250 mL to about 150 L, about 250 mL to about 100 L, about 250 mL to about 50 L, about 250 mL to about 10 L, about 250 mL to about 5 L, about 250 mL to about 1 L, about 250 mL to about 500 mL, about 500 mL to about 500 L, about 500 mL to about 450 L, about 500 mL to about 400 L, about 500 mL to about 350 L, about 500 mL to about 300 L, about 500 mL to about 250 L, about 500 mL to about 200 L, about 500 mL to about 150 L, about 500 mL to about 100 L, about 500 mL to about 50 L, about 500 mL to about 10 L, about 500 mL to about 5 L, about 500 mL to about 1 L, about 1 L to about 500 L, about 1 L to about 450 L, about 1 L to about 400 L, about 1 L to about 350 L, about 1 L to about 300 L, about 1 L to about 250 L, about 1 L to about 200 L, about 1 L to about 150 L, about 1 L to about 100 L, about 1 L to about 50 L, about 1 L to about 10 L, about 1 L to about 5 L, about 5 L to about 500 L, about 5 L to about 450 L, about 5 L to about 400 L, about 5 L to about 350 L, about 5 L to about 300 L, about 5 L to about 250 L, about 5 L to about 200 L, about 5 L to about 150 L, about 5 L to about 100 L, about 5 L to about 50 L, about 5 L to about 10 L, about 10 L to about 500 L, about 10 L to about 450 L, about 10 L to about 400 L, about 10 L to about 350 L, about 10 L to about 300 L, about 10 L to about 250 L, about 10 L to about 200 L, about 10 L to about 150 L, about 10 L to about 100 L, about 10 L to about 50 L, about 50 L to about 500 L, about 50 L to about 450 L, about 50 L to about 400 L, about 50 L to about 350 L, about 50 L to about 300 L, about 50 L to about 250 L, about 50 L to about 200 L, about 50 L to about 150 L, about 50 L to about 100 L, about 100 L to about 500 L, about 100 L to about 450 L, about 100 L to about 400 L, about 100 L to about 350 L, about 100 L to about 300 L, about 100 L to about 250 L, about 100 L to about 200 L, about 100 L to about 150 L, about 150 L to about 500 L, about 150 L to about 450 L, about 150 L to about 400 L, about 150 L to about 350 L, about 150 L to about 300 L, about 150 L to about 250 L, about 150 L to about 200 L, about 200 L to about 500 L, about 200 L to about 450 L, about 200 L to about 400 L, about 200 L to about 350 L, about 200 L to about 300 L, about 200 L to about 250 L, about 250 L to about 500 L, about 250 L to about 450 L, about 250 L to about 400 L, about 250 L to about 350 L, about 250 L to about 300 L, about 300 L to about 500 L, about 300 L to about 450 L, about 300 L to about 400 L, about 300 L to about 350 L, about 350 L to about 500 L, about 350 L to about 450 L, about 350 L to about 400 L, about 400 L to about 500 L, about 400 L to about 450 L, or about 450 to about 500 L.

In some embodiments, the culturing can include shifting the temperature of the recombinant protein production medium during the period of time. In some embodiments, the temperature of the recombinant protein production medium after the shifting is about 28° C. to about 35° C. (e.g., about 32° C. to about 35° C.). In some embodiments, the shifting of the temperature is performed at about 1 days to about 7 days after the start of culturing (e.g., about 4 days to about 7 days after the start of culturing, or about 5 days to about 7 days after the start of culturing).

In some embodiments, the culturing includes maintaining a dissolved O₂ (dO₂) level of about 5% to about 60% (e.g., about 20% to about 50%) in the recombinant protein production medium. In some embodiments, the culturing includes maintaining a dissolved O₂ (dO₂) level of greater than about 30% in the recombinant protein production medium. In some embodiments, the culturing includes maintaining a dissolved O₂ (dO₂) level of about 30% to about 55% in the recombinant protein production medium.

In some embodiments, the culturing includes agitating the recombinant protein production medium. In some embodiments, the agitating is performed at a rotary agitation rate of about 100 revolutions per minute (RPM) to about 800 RPM.

### Fed Batch Culturing

Fed batch culturing a cell in a vessel (e.g., a fed-batch bioreactor or fermentor) includes, over the majority of the culturing period, the addition (e.g., periodic or continuous addition) to the recombinant protein culture medium of a volume of a feed liquid culture medium, without substantial or significant removal of the recombinant protein production medium. In some embodiments, the adding of the feed liquid culture medium is performed continuously (e.g., at a rate that adds a volume of between about 1% to about 500% (e.g., about 1% to about 450%, about 1% to about 400%, about 1% to about 350%, about 1% to about 300%, about 1% to about 25%, about 1% to about 200%, about 1% to about 150%, about 1% to about 100%, about 1% to about 50%, about 1% to about 25%, about 10% to about 500%, about 10% to about 450%, about 10% to about 400%, about 10% to about 350%, about 10% to about 300%, about 10% to about 25%, about 10% to about 200%, about 10% to about 150%, about 10% to about 100%, about 10% to about 50%, about 10% to about 25%, about 25% to about 500%, about 25% to about 45%, about 25% to about 400%, about 25% to about 350%, about 25% to about 300%, about 25% to about 250%, about 25% to about 200%, about 25% to about 150%, about 25% to about 100%, about 25% to about 50%, about 50% to about 500%, about 50% to about 450%, about 50% to about 400%, about 50% to about 350%, about 50% to about 300%, about 50% to about 25%, about 50% to about 200%, about 50% to about 150%, about 50% to about 100%, about 100% to about 500%, about 100% to about 450%, about 100% to about 400%, about 100% to about 350%, about 100% to about 300%, about 100% to about 250%, about 100% to about 200%, about 100% to about 150%, about 150% to about 500%, about 150% to about 450%, about 150% to about 400%, about 150% to about 350%, about 150% to about 300%, about 150% to about 250%, about 150% to about 200%, about 200% to about 500%, about 200% to about 45%, about 200% to about 400%, about 200% to about 350%, about 200% to about 300%, about 200% to about 250%, about 250% to about 500%, about 250% to about 450%, about 250% to about 400%, about 250% to about 350%, about 250% to about 300%, about 300% to about 500%, about 300% to about 450%, about 300% to about 400%, about 300% to about 350%, about 350% to about 500%, about 350% to about 450%, about 350% to about 400%, about 400% to about 500% or about 450% to about 500%) of the volume of the recombinant protein production medium at the start of culturing, over the entire time of culturing.

Skilled practitioners will appreciate that the liquid culture medium first contacted with recombinant cells to prepare a recombinant protein production medium, and the feed liquid culture medium added to an already obtained recombinant protein production medium can be the same or different.

In some embodiments, a minimal volume of the recombinant protein production medium is removed during the culturing for testing of the culture, e.g., by a mechanical system. In some embodiments, the volume of feed liquid culture medium is added to the recombinant protein production medium in an automated fashion, e.g., by perfusion pump.

In some embodiments of these methods, once the cell culture achieves a threshold optical density (e.g., an OD₆₀₀ of about 20, 30, 40, 50, 60, 70, 80, 90, 100, 110, 120, 130, 140, 150, 160, 170, 180, 190, or 200), a feed liquid culture medium including phosphate can be added to the recombinant protein production medium.

In some embodiments of these methods, a feed liquid culture medium including phosphate is added to the recombinant protein production medium once the elapsed fermentation is about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the phosphate is phosphoric acid. In some embodiments, the phosphoric acid is 85% phosphoric acid.

In some embodiments, the phosphate is added to the recombinant protein production medium at a rate of about 0.1 g phosphate/L/hour to about 30 g phosphate/L/hour, about 0.1 g phosphate/L/hour to about 25 g phosphate/L/hour, about 0.1 g phosphate/L/hour to about 20 g phosphate/L/hour, about 0.1 g phosphate/L/hour to about 15 g phosphate/L/hour, about 1 g phosphate/L/hour to about 20 g phosphate/L/hour, about 1 g phosphate/L/hour to about 15 g phosphate/L/hour, about 5 g phosphate/L/hour to about 20 g phosphate/L/hour, about 5 g phosphate/L/hour to about 15 g phosphate/L/hour, about 10 g phosphate/L/hour to about 20 g phosphate/L/hour, about 10 g phosphate/L/hour to about 15 g phosphate/L/hour, about 0.2 g phosphate/L/hour to about 5.0 g phosphate/L/hour, about 0.2 g phosphate/L/hour to about 4.5 g phosphate/L/hour, about 0.2 g phosphate/L/hour to about 4.0 g phosphate/L/hour, about 0.2 g phosphate/L/hour to about 3.5 g phosphate/L/hour, about 0.2 g phosphate/L/hour to about 3.0 g phosphate/L/hour, about 0.2 g phosphate/L/hour to about 2.5 g phosphate/L/hour, about 0.2 g phosphate/L/hour to about 2.0 g phosphate/L/hour, about 0.2 g phosphate/L/hour to about 1.5 g phosphate/L/hour, about 0.2 g phosphate/L/hour to about 1.0 g phosphate/L/hour, about 0.2 g phosphate/L/hour to about 0.5 g phosphate/L/hour, about 0.5 g phosphate/L/hour to about 5.0 g phosphate/L/hour, about 0.5 g phosphate/L/hour to about 4.5 g phosphate/L/hour, about 0.5 g phosphate/L/hour to about 4.0 g phosphate/L/hour, about 0.5 g phosphate/L/hour to about 3.5 g phosphate/L/hour, about 0.5 g phosphate/L/hour to about 3.0 g phosphate/L/hour, about 0.5 g phosphate/L/hour to about 2.5 g phosphate/L/hour, about 0.5 g phosphate/L/hour to about 2.0 g phosphate/L/hour, about 0.5 g phosphate/L/hour to about 1.5 g phosphate/L/hour, about 0.5 g phosphate/L/hour to about 1.0 g phosphate/L/hour, about 1.0 g phosphate/L/hour to about 5.0 g phosphate/L/hour, about 1.0 g phosphate/L/hour to about 4.5 g phosphate/L/hour, about 1.0 g phosphate/L/hour to about 4.0 g phosphate/L/hour, about 1.0 g phosphate/L/hour to about 3.5 g phosphate/L/hour, about 1.0 g phosphate/L/hour to about 3.0 g phosphate/L/hour, about 1.0 g phosphate/L/hour to about 2.5 g phosphate/L/hour, about 1.0 g phosphate/L/hour to about 2.0 g phosphate/L/hour, about 1.0 g phosphate/L/hour to about 1.5 g phosphate/L/hour, about 1.5 g phosphate/L/hour to about 5.0 g phosphate/L/hour, about 1.5 g phosphate/L/hour to about 4.5 g phosphate/L/hour, about 1.5 g phosphate/L/hour to about 4.0 g phosphate/L/hour, about 1.5 g phosphate/L/hour to about 3.5 g phosphate/L/hour, about 1.5 g phosphate/L/hour to about 3.0 g phosphate/L/hour, about 1.5 g phosphate/L/hour to about 2.5 g phosphate/L/hour, about 1.5 g phosphate/L/hour to about 2.0 g phosphate/L/hour, about 2.0 g phosphate/L/hour to about 5.0 g phosphate/L/hour, about 2.0 g phosphate/L/hour to about 4.5 g phosphate/L/hour, about 2.0 g phosphate/L/hour to about 4.0 g phosphate/L/hour, about 2.0 g phosphate/L/hour to about 3.5 g phosphate/L/hour, about 2.0 g phosphate/L/hour to about 3.0 g phosphate/L/hour, about 2.0 g phosphate/L/hour to about 2.5 g phosphate/L/hour, about 2.5 g phosphate/L/hour to about 5.0 g phosphate/L/hour, about 2.5 g phosphate/L/hour to about 4.5 g phosphate/L/hour, about 2.5 g phosphate/L/hour to about 4.0 g phosphate/L/hour, about 2.5 g phosphate/L/hour to about 3.5 g phosphate/L/hour, about 2.5 g phosphate/L/hour to about 3.0 g phosphate/L/hour, about 3.0 g phosphate/L/hour to about 5.0 g phosphate/L/hour, about 3.0 g phosphate/L/hour to about 4.5 g phosphate/L/hour, about 3.0 g phosphate/L/hour to about 4.0 g phosphate/L/hour, about 3.0 g phosphate/L/hour to about 3.5 g phosphate/L/hour, about 3.5 g phosphate/L/hour to about 5.0 g phosphate/L/hour, about 3.5 g phosphate/L/hour to about 4.5 g phosphate/L/hour, about 3.5 g phosphate/L/hour to about 4.0 g phosphate/L/hour, about 4.0 g phosphate/L/hour to about 5.0 g phosphate/L/hour, about 4.0 g phosphate/L/hour to about 4.5 g phosphate/L/hour, or about 4.5 g phosphate/L/hour to about 5.0 g phosphate/L/hour, and can, e.g., be continued for a period of about 1 hour to about 60 hours (e.g., about 1 hour to about 58 hours, about 1 hour to about 56 hours, about 1 hour to about 54 hours, about 1 hour to about 52 hours, about 1 hour to about 50 hours, about 1 hour to about 48 hours, about 1 hour to about 46 hours, about 1 hour to about 44 hours, about 1 hour to about 42 hours, about 1 hour to about 40 hours, about 1 hour to about 38 hours, about 1 hour to about 36 hours, about 1 hour to about 34 hours, about 1 hour to about 32 hours, about 1 hour to about 30 hours, about 1 hour to about 28 hours, about 1 hour to about 26 hours, about 1 hour to about 24 hours, about 1 hour to about 22 hours, about 1 hour to about 20 hours, about 1 hour to about 18 hours, about 1 hour to about 16 hours, about 1 hour to about 14 hours, about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 32 hours, about 2 hours to about 30 hours, about 2 hours to about 28 hours, about 2 hours to about 26 hours, about 2 hours to about 24 hours, about 2 hours to about 22 hours, about 2 hours to about 20 hours, about 2 hours to about 18 hours, about 2 hours to about 16 hours, about 2 hours to about 14 hours, about 2 hours to about 12 hours, about 2 hours to about 10 hours, about 2 hours to about 8 hours, about 2 hours to about 6 hours, about 2 hours to about 4 hours, about 4 hours to about 32 hours, about 4 hours to about 30 hours, about 4 hours to about 28 hours, about 4 hours to about 26 hours, about 4 hours to about 24 hours, about 4 hours to about 22 hours, about 4 hours to about 20 hours, about 4 hours to about 18 hours, about 4 hours to about 16 hours, about 4 hours to about 14 hours, about 4 hours to about 12 hours, about 4 hours to about 10 hours, about 4 hours to about 8 hours, about 4 hours to about 6 hours, about 6 hours to about 32 hours, about 6 hours to about 30 hours, about 6 hours to about 28 hours, about 6 hours to about 26 hours, about 6 hours to about 24 hours, about 6 hours to about 22 hours, about 6 hours to about 20 hours, about 6 hours to about 18 hours, about 6 hours to about 16 hours, about 6 hours to about 14 hours, about 6 hours to about 12 hours, about 6 hours to about 10 hours, about 6 hours to about 8 hours, about 8 hours to about 32 hours, about 8 hours to about 30 hours, about 8 hours to about 28 hours, about 8 hours to about 26 hours, about 8 hours to about 24 hours, about 8 hours to about 22 hours, about 8 hours to about 20 hours, about 8 hours to about 18 hours, about 8 hours to about 16 hours, about 8 hours to about 14 hours, about 8 hours to about 12 hours, about 8 hours to about 10 hours, about 10 hours to about 32 hours, about 10 hours to about 30 hours, about 10 hours to about 28 hours, about 10 hours to about 26 hours, about 10 hours to about 24 hours, about 10 hours to about 22 hours, about 10 hours to about 20 hours, about 10 hours to about 18 hours, about 10 hours to about 16 hours, about 10 hours to about 14 hours, about 10 hours to about 12 hours, about 12 hours to about 32 hours, about 12 hours to about 30 hours, about 12 hours to about 28 hours, about 12 hours to about 26 hours, about 12 hours to about 24 hours, about 12 hours to about 22 hours, about 12 hours to about 20 hours, about 12 hours to about 18 hours, about 12 hours to about 16 hours, about 12 hours to about 14 hours, about 14 hours to about 32 hours, about 14 hours to about 30 hours, about 14 hours to about 28 hours, about 14 hours to about 26 hours, about 14 hours to about 24 hours, about 14 hours to about 22 hours, about 14 hours to about 20 hours, about 14 hours to about 18 hours, about 14 hours to about 16 hours, about 16 hours to about 32 hours, about 16 hours to about 30 hours, about 16 hours to about 28 hours, about 16 hours to about 26 hours, about 16 hours to about 24 hours, about 16 hours to about 22 hours, about 16 hours to about 20 hours, about 16 hours to about 18 hours, about 18 hours to about 32 hours, about 18 hours to about 30 hours, about 18 hours to about 28 hours, about 18 hours to about 26 hours, about 18 hours to about 24 hours, about 18 hours to about 22 hours, about 18 hours to about 20 hours, about 20 hours to about 32 hours, about 20 hours to about 30 hours, about 20 hours to about 28 hours, about 20 hours to about 26 hours, about 20 hours to about 24 hours, about 20 hours to about 22 hours, about 22 hours to about 32 hours, about 22 hours to about 30 hours, about 22 hours to about 28 hours, about 22 hours to about 26 hours, about 22 hours to about 24 hours, about 24 hours to about 32 hours, about 24 hours to about 30 hours, about 24 hours to about 28 hours, about 24 hours to about 26 hours, about 26 hours to about 32 hours, about 26 hours to about 30 hours, about 26 hours to about 28 hours, about 28 hours to about 32 hours, about 28 hours to about 30 hours, about 30 hours to about 32 hours, about 24 hour to about 50 hours, about 30 hour to about 50 hours, about 36 hour to about 50 hours, about 24 hour to about 48 hours, about 24 hour to about 36 hours, or about 36 hour to about 48 hours).

In some examples, the adding of the feed liquid culture medium is initiated after about the first 10 minutes, the first 20 minutes, the first 30 minutes, the first 40 minutes, the first 50 minutes, the first 1 hour, the first 2 hours, the first 3 hours, the first 4 hours, the first 5 hours, the first 6 hours, the first 7 hours, the first 8 hours, the first 9 hours, the first 10 hours, the first 11 hours, the first 12 hours, the first 14 hours, the first 16 hours, the first 18 hours, the first 20 hours, the first 22 hours, the first 24 hours, the first 36 hours, or the first 48 hours) after the start of culturing.

In some embodiments of these methods, a feed liquid culture medium including a magnesium ion is added to the recombinant protein production medium. In some embodiments, the magnesium ion is provided in a magnesium salt. In some embodiments, the magnesium salt is a solid. In some embodiments, the magnesium salt is provided in a solution (e.g. water).

In some embodiments, the magnesium ion is added to the recombinant protein production medium at a rate of about 0.1 g Mg²⁺/L/hour to about 30 g Mg²⁺/L/hour, about 0.1 g Mg²⁺/L/hour to about 25 g Mg²⁺/L/hour, about 0.1 g Mg²⁺/L/hour to about 20 g Mg²⁺/L/hour, about 0.1 g Mg²⁺/L/hour to about 15 g Mg²⁺/L/hour, about 1 g Mg²⁺/L/hour to about 20 g Mg²⁺/L/hour, about 1 g Mg²⁺/L/hour to about 15 g Mg²⁺/L/hour, about 5 g Mg²⁺/L/hour to about 20 g Mg²⁺/L/hour, about 5 g Mg²⁺/L/hour to about 15 g Mg²⁺/L/hour, about 10 g Mg²⁺/L/hour to about 20 g Mg²⁺/L/hour, about 10 g Mg²⁺/L/hour to about 15 g Mg²⁺/L/hour, about 0.2 g Mg²⁺/L/hour to about 5.0 g Mg²⁺/L/hour, about 0.2 g Mg²⁺/L/hour to about 4.5 g Mg²⁺/L/hour, about 0.2 g Mg²⁺/L/hour to about 4.0 g Mg²⁺/L/hour, about 0.2 g Mg²⁺/L/hour to about 3.5 g Mg²⁺/L/hour, about 0.2 g Mg²⁺/L/hour to about 3.0 g Mg²⁺/L/hour, about 0.2 g Mg²⁺/L/hour to about 2.5 g Mg²⁺/L/hour, about 0.2 g Mg²⁺/L/hour to about 2.0 g Mg²⁺/L/hour, about 0.2 g Mg²⁺/L/hour to about 1.5 g Mg²⁺/L/hour, about 0.2 g Mg²⁺/L/hour to about 1.0 g Mg²⁺/L/hour, about 0.2 g Mg²⁺/L/hour to about 0.5 g Mg²⁺/L/hour, about 0.5 g Mg²⁺/L/hour to about 5.0 g Mg²⁺/L/hour, about 0.5 g Mg²⁺/L/hour to about 4.5 g Mg²⁺/L/hour, about 0.5 g Mg²⁺/L/hour to about 4.0 g Mg²⁺/L/hour, about 0.5 g Mg²⁺/L/hour to about 3.5 g Mg²⁺/L/hour, about 0.5 g Mg²⁺/L/hour to about 3.0 g Mg²⁺/L/hour, about 0.5 g Mg²⁺/L/hour to about 2.5 g Mg²⁺/L/hour, about 0.5 g Mg²⁺/L/hour to about 2.0 g Mg²⁺/L/hour, about 0.5 g Mg²⁺/L/hour to about 1.5 g Mg²⁺/L/hour, about 0.5 g Mg²⁺/L/hour to about 1.0 g Mg²⁺/L/hour, about 1.0 g Mg²⁺/L/hour to about 5.0 g Mg²⁺/L/hour, about 1.0 g Mg²⁺/L/hour to about 4.5 g Mg²⁺/L/hour, about 1.0 g Mg²⁺/L/hour to about 4.0 g Mg²⁺/L/hour, about 1.0 g Mg²⁺/L/hour to about 3.5 g Mg²⁺/L/hour, about 1.0 g Mg²⁺/L/hour to about 3.0 g Mg²⁺/L/hour, about 1.0 g Mg²⁺/L/hour to about 2.5 g Mg²⁺/L/hour, about 1.0 g Mg²⁺/L/hour to about 2.0 g Mg²⁺/L/hour, about 1.0 g Mg²⁺/L/hour to about 1.5 g Mg²⁺/L/hour, about 1.5 g Mg²⁺/L/hour to about 5.0 g Mg²⁺/L/hour, about 1.5 g Mg²⁺/L/hour to about 4.5 g Mg²⁺/L/hour, about 1.5 g Mg²⁺/L/hour to about 4.0 g Mg²⁺/L/hour, about 1.5 g Mg²⁺/L/hour to about 3.5 g Mg²⁺/L/hour, about 1.5 g Mg²⁺/L/hour to about 3.0 g Mg²⁺/L/hour, about 1.5 g Mg²⁺/L/hour to about 2.5 g Mg²⁺/L/hour, about 1.5 g Mg²⁺/L/hour to about 2.0 g Mg²⁺/L/hour, about 2.0 g Mg²⁺/L/hour to about 5.0 g Mg²⁺/L/hour, about 2.0 g Mg²⁺/L/hour to about 4.5 g Mg²⁺/L/hour, about 2.0 g Mg²⁺/L/hour to about 4.0 g Mg²⁺/L/hour, about 2.0 g Mg²⁺/L/hour to about 3.5 g Mg²⁺/L/hour, about 2.0 g Mg²⁺/L/hour to about 3.0 g Mg²⁺/L/hour, about 2.0 g Mg²⁺/L/hour to about 2.5 g Mg²⁺/L/hour, about 2.5 g Mg²⁺/L/hour to about 5.0 g Mg²⁺/L/hour, about 2.5 g Mg²⁺/L/hour to about 4.5 g Mg²⁺/L/hour, about 2.5 g Mg²⁺/L/hour to about 4.0 g Mg²⁺/L/hour, about 2.5 g Mg²⁺/L/hour to about 3.5 g Mg²⁺/L/hour, about 2.5 g Mg²⁺/L/hour to about 3.0 g Mg²⁺/L/hour, about 3.0 g Mg²⁺/L/hour to about 5.0 g Mg²⁺/L/hour, about 3.0 g Mg²⁺/L/hour to about 4.5 g Mg²⁺/L/hour, about 3.0 g Mg²⁺/L/hour to about 4.0 g Mg²⁺/L/hour, about 3.0 g Mg²⁺/L/hour to about 3.5 g Mg²⁺/L/hour, about 3.5 g Mg²⁺/L/hour to about 5.0 g Mg²⁺/L/hour, about 3.5 g Mg²⁺/L/hour to about 4.5 g Mg²⁺/L/hour, about 3.5 g Mg²⁺/L/hour to about 4.0 g Mg²⁺/L/hour, about 4.0 g Mg²⁺/L/hour to about 5.0 g Mg²⁺/L/hour, about 4.0 g Mg²⁺/L/hour to about 4.5 g Mg²⁺/L/hour, or about 4.5 g Mg²⁺/L/hour to about 5.0 g Mg²⁺/L/hour, and can, e.g., be continued for a period of about 1 hour to about 60 hours (e.g., about 1 hour to about 58 hours, about 1 hour to about 56 hours, about 1 hour to about 54 hours, about 1 hour to about 52 hours, about 1 hour to about 50 hours, about 1 hour to about 48 hours, about 1 hour to about 46 hours, about 1 hour to about 44 hours, about 1 hour to about 42 hours, about 1 hour to about 40 hours, about 1 hour to about 38 hours, about 1 hour to about 36 hours, about 1 hour to about 34 hours, about 1 hour to about 32 hours, about 1 hour to about 30 hours, about 1 hour to about 28 hours, about 1 hour to about 26 hours, about 1 hour to about 24 hours, about 1 hour to about 22 hours, about 1 hour to about 20 hours, about 1 hour to about 18 hours, about 1 hour to about 16 hours, about 1 hour to about 14 hours, about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 32 hours, about 2 hours to about 30 hours, about 2 hours to about 28 hours, about 2 hours to about 26 hours, about 2 hours to about 24 hours, about 2 hours to about 22 hours, about 2 hours to about 20 hours, about 2 hours to about 18 hours, about 2 hours to about 16 hours, about 2 hours to about 14 hours, about 2 hours to about 12 hours, about 2 hours to about 10 hours, about 2 hours to about 8 hours, about 2 hours to about 6 hours, about 2 hours to about 4 hours, about 4 hours to about 32 hours, about 4 hours to about 30 hours, about 4 hours to about 28 hours, about 4 hours to about 26 hours, about 4 hours to about 24 hours, about 4 hours to about 22 hours, about 4 hours to about 20 hours, about 4 hours to about 18 hours, about 4 hours to about 16 hours, about 4 hours to about 14 hours, about 4 hours to about 12 hours, about 4 hours to about 10 hours, about 4 hours to about 8 hours, about 4 hours to about 6 hours, about 6 hours to about 32 hours, about 6 hours to about 30 hours, about 6 hours to about 28 hours, about 6 hours to about 26 hours, about 6 hours to about 24 hours, about 6 hours to about 22 hours, about 6 hours to about 20 hours, about 6 hours to about 18 hours, about 6 hours to about 16 hours, about 6 hours to about 14 hours, about 6 hours to about 12 hours, about 6 hours to about 10 hours, about 6 hours to about 8 hours, about 8 hours to about 32 hours, about 8 hours to about 30 hours, about 8 hours to about 28 hours, about 8 hours to about 26 hours, about 8 hours to about 24 hours, about 8 hours to about 22 hours, about 8 hours to about 20 hours, about 8 hours to about 18 hours, about 8 hours to about 16 hours, about 8 hours to about 14 hours, about 8 hours to about 12 hours, about 8 hours to about 10 hours, about 10 hours to about 32 hours, about 10 hours to about 30 hours, about 10 hours to about 28 hours, about 10 hours to about 26 hours, about 10 hours to about 24 hours, about 10 hours to about 22 hours, about 10 hours to about 20 hours, about 10 hours to about 18 hours, about 10 hours to about 16 hours, about 10 hours to about 14 hours, about 10 hours to about 12 hours, about 12 hours to about 32 hours, about 12 hours to about 30 hours, about 12 hours to about 28 hours, about 12 hours to about 26 hours, about 12 hours to about 24 hours, about 12 hours to about 22 hours, about 12 hours to about 20 hours, about 12 hours to about 18 hours, about 12 hours to about 16 hours, about 12 hours to about 14 hours, about 14 hours to about 32 hours, about 14 hours to about 30 hours, about 14 hours to about 28 hours, about 14 hours to about 26 hours, about 14 hours to about 24 hours, about 14 hours to about 22 hours, about 14 hours to about 20 hours, about 14 hours to about 18 hours, about 14 hours to about 16 hours, about 16 hours to about 32 hours, about 16 hours to about 30 hours, about 16 hours to about 28 hours, about 16 hours to about 26 hours, about 16 hours to about 24 hours, about 16 hours to about 22 hours, about 16 hours to about 20 hours, about 16 hours to about 18 hours, about 18 hours to about 32 hours, about 18 hours to about 30 hours, about 18 hours to about 28 hours, about 18 hours to about 26 hours, about 18 hours to about 24 hours, about 18 hours to about 22 hours, about 18 hours to about 20 hours, about 20 hours to about 32 hours, about 20 hours to about 30 hours, about 20 hours to about 28 hours, about 20 hours to about 26 hours, about 20 hours to about 24 hours, about 20 hours to about 22 hours, about 22 hours to about 32 hours, about 22 hours to about 30 hours, about 22 hours to about 28 hours, about 22 hours to about 26 hours, about 22 hours to about 24 hours, about 24 hours to about 32 hours, about 24 hours to about 30 hours, about 24 hours to about 28 hours, about 24 hours to about 26 hours, about 26 hours to about 32 hours, about 26 hours to about 30 hours, about 26 hours to about 28 hours, about 28 hours to about 32 hours, about 28 hours to about 30 hours, about 30 hours to about 32 hours, about 24 hour to about 50 hours, about 30 hour to about 50 hours, about 36 hour to about 50 hours, about 24 hour to about 48 hours, about 24 hour to about 36 hours, or about 36 hour to about 48 hours).

In some examples, the adding of the feed liquid culture medium is initiated after about the first 10 minutes, the first 20 minutes, the first 30 minutes, the first 40 minutes, the first 50 minutes, the first 1 hour, the first 2 hours, the first 3 hours, the first 4 hours, the first 5 hours, the first 6 hours, the first 7 hours, the first 8 hours, the first 9 hours, the first 10 hours, the first 11 hours, the first 12 hours, the first 14 hours, the first 16 hours, the first 18 hours, the first 20 hours, the first 22 hours, the first 24 hours, the first 36 hours, or the first 48 hours) after the start of culturing.

In some embodiments, the magnesium ion is included in the phosphate feed liquid culture medium. In some embodiments, the feed liquid culture medium contains an amount of phosphate and an amount of a magnesium salt in order to provide the desired rate of addition of both phosphate and Mg²⁺ to the recombinant protein production medium.

### Bioreactors and Fermentors

In some examples, the culturing is performed using a shake flask. In some examples, the culturing is performed using a bioreactor (e.g., a fed batch fermentor). As is described in the art, in some embodiments, a bioreactor (e.g., fermentor) is equipped with several ports for, e.g., the removal of a fluid (e.g., a recombinant protein production medium, e.g., that can be substantially free of recombinant bacteria or that includes recombinant bacteria) or the addition of a fluid (e.g., a feed liquid culture medium, a basic or acidic solution for regulating the pH, and/or a fluid including phosphate). A bioreactor (e.g., fermentor) can also be equipped with a pH monitor, a dissolved oxygen monitor, a dissolved CO2 monitor, one or more gas spargers, and a means for agitating the recombinant protein production medium (e.g., a propeller). A variety of different bioreactors (e.g., fermenters) are commercially available. In some embodiments, a bioreactor is equipped with a mechanical device that is capable of removing a volume of recombinant protein production medium from the bioreactor and optionally, a mechanical device for adding a volume of a feed liquid culture medium).

In some embodiments, the internal volume of a bioreactor (e.g., bioreactor or fermentor) is about 100 mL to about 1,000 L, about 100 mL to about 750 L, about 100 mL to about 500 L, about 100 mL to about 250 L, about 100 mL to about 100 L, about 100 mL to about 10 L, about 100 mL to about 1 L, about 100 mL to about 500 mL, about 500 mL to about 1,000 L, about 500 mL to about 750 L, about 500 mL to about 500 L, about 500 mL to about 250 L, about 500 mL to about 100 L, about 500 mL to about 10 L, about 500 mL to about 1 L, about 1 L to about 1,000 L, about 1 L to about 750 L, about 1 L to about 500 L, about 1 L to about 250 L, about 1 L to about 100 L, about 1 L to about 10 L, about 10 L to about 1,000 L, about 10 L to about 750 L, about 10 L to about 500 L, about 10 L to about 250 L, about 10 L to about 100 L, about 100 L to about 1,000 L, about 100 L to about 750 L, about 100 L to about 500 L, about 100 L to about 250 L, about 250 L to about 1,000 L, about 250 L to about 750 L, about 250 L to about 500 L, about 500 L to about 1,000 L, about 500 L to about 750 L, or about 750 L to about 1,000 L.

### Period of Time

In some examples, the culturing is performed for about 1 day to about 7 days, about 1 day to about 156 hours, about 1 day to about 6 days, about 1 day to about 132 hours, about 1 day to about 5 days, about 1 day to about 108 hours, about 1 day to about 4 days, about 1 day to about 84 hours, about 1 day to about 3 days, about 1 day to about 60 hours, about 1 day to about 2 days, about 1 day to about 36 hours, about 36 hours to about 7 days, about 36 hours to about 156 hours, about 36 hours to about 6 days, about 36 hours to about 132 hours, about 36 hours to about 5 days, about 36 hours to about 108 hours, about 36 hours to about 4 days, about 36 hours to about 84 hours, about 36 hours to about 3 days, about 36 hours to about 60 hours, about 36 hours to about 2 days, about 2 days to about 7 days, about 2 days to about 156 hours, about 2 days to about 6 days, about 2 days to about 132 hours, about 2 days to about 5 days, about 2 days to about 108 hours, about 2 days to about 4 days, about 2 days to about 84 hours, about 2 days to about 3 days, about 2 days to about 60 hours, about 60 hours to about 7 days, about 60 hours to about 156 hours, about 60 hours to about 6 days, about 60 hours to about 132 hours, about 60 hours to about 5 days, about 60 hours to about 108 hours, about 60 hours to about 4 days, about 60 hours to about 84 hours, about 60 hours to about 3 days, about 3 days to about 7 days, about 3 days to about 156 hours, about 3 days to about 6 days, about 3 days to about 132 hours, about 3 days to about 5 days, about 3 days to about 108 hours, about 3 days to about 4 days, about 3 days to about 84 hours, about 84 hours to about 7 days, about 84 hours to about 156 hours, about 84 hours to about 6 days, about 84 hours to about 132 hours, about 84 hours to about 5 days, about 84 hours to about 108 hours, about 84 hours to about 4 days, about 4 days to about 7 days, about 4 days to about 156 hours, about 4 days to about 6 days, about 4 days to about 132 hours, about 4 days to about 5 days, about 4 days to about 108 hours, about 108 hours to about 7 days, about 108 hours to about 156 hours, about 108 hours to about 6 days, about 108 hours to about 132 hours, about 108 hours to about 5 days, about 5 days to about 7 days, about 5 days to about 156 hours, about 5 days to about 6 days, about 5 days to about 132 hours, about 132 hours to about 7 days, about 132 hours to about 156 hours, about 132 hours to about 6 days, about 6 days to about 7 days, about 6 days to about 156 hours, or about 156 hours to about 7 days.

### Temperature and Temperature Shift

In some embodiments, the temperature of the recombinant protein production medium during the culturing step is about 25° C. to about 37° C. (e.g., about 25° C. to about 35° C., about 25° C. to about 30° C., about 30° C. to about 37° C., about 30° C. to about 35° C., or about 35° C. to about 37° C.). Skilled practitioners will appreciate that the temperature of the recombinant protein production medium can be changed at a specific time point(s) during the culturing step. In non-limiting examples, the temperature of the recombinant protein production medium is changed or shifted (e.g., increased or decreased) at about 1 day, about 2 days, about 3 days, about 4 days, about 5 days, about 6 days, or about 7 days after the start of culturing. For example, the shifting of the temperature is performed at about 2 days to about 7 days (e.g., about 2 days to about 6 days, about 2 days to about 5 days, about 2 days to about 4 days, about 3 days to about 7 days, about 3 days to about 6 days, about 3 days to about 5 days, about 4 days to about 7 days, about 4 days to about 6 days, or about 5 days to about 7 days) after the start of culturing.

Dissolved Oxygen (dO₂)

The culturing can include maintaining in the recombinant protein production medium a dissolved oxygen (dO₂) level. In some embodiments, the dO₂ level is maintained at about 5% to about 55% (e.g., about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 55%, about 15% to about 50%, about 15% to about 45%, about 15% to about 40%, about 15% to about 35%, about 15% to about 30%, about 15% to about 25%, about 15% to about 20%, about 20% to about 55%, about 20% to about 50%, about 20% to about 45%, about 20% to about 40%, about 20% to about 35%, about 20% to about 30%, about 20% to about 25%, about 25% to about 55%, about 25% to about 50%, about 25% to about 45%, about 25% to about 40%, about 25% to about 35%, about 25% to about 30%, about 30% to about 55%, about 30% to about 50%, about 30% to about 45%, about 30% to about 40%, about 30% to about 35%, about 35% to about 55%, about 35% to about 50%, about 35% to about 45%, about 35% to about 40%, about 40% to about 55%, about 40% to about 50%, about 40% to about 45%, about 45% to about 55%, about 45% to about 50%, or about 50% to about 55%). In some embodiments, the dO₂ level is maintained at greater than about 10%, greater than about 15%, greater than about 20%, greater than about 25%, greater than about 30%, greater than about 35%, greater than about 40%, greater than about 45%, or greater than about 50%.

### Agitation

In some embodiments, the culturing includes agitating the liquid culture medium and/or the recombinant protein production medium. In some examples of culturing, the agitating is performed at a frequency of about 100 RPM to about 800 RPM, about 100 RPM to about 750 RPM, about 100 RPM to about 700 RPM, about 100 RPM to about 650 RPM, about 100 RPM to about 600 RPM, about 100 RPM to about 550 RPM, about 100 RPM to about 500 RPM, about 100 RPM to about 450 RPM, about 100 RPM to about 400 RPM, about 100 RPM to about 350 RPM, about 100 RPM to about 300 RPM, about 100 RPM to about 250 RPM, about 100 RPM to about 200 RPM, about 100 RPM to about 150 RPM, about 150 RPM to about 800 RPM, about 150 RPM to about 750 RPM, about 150 RPM to about 700 RPM, about 150 RPM to about 650 RPM, about 150 RPM to about 600 RPM, about 150 RPM to about 550 RPM, about 150 RPM to about 500 RPM, about 150 RPM to about 450 RPM, about 150 RPM to about 400 RPM, about 150 RPM to about 350 RPM, about 150 RPM to about 300 RPM, about 150 RPM to about 250 RPM, about 150 RPM to about 200 RPM, about 200 RPM to about 800 RPM, about 200 RPM to about 750 RPM, about 200 RPM to about 700 RPM, about 200 RPM to about 650 RPM, about 200 RPM to about 600 RPM, about 200 RPM to about 550 RPM, about 200 RPM to about 500 RPM, about 200 RPM to about 450 RPM, about 200 RPM to about 400 RPM, about 200 RPM to about 350 RPM, about 200 RPM to about 300 RPM, about 200 RPM to about 250 RPM. about 250 RPM to about 800 RPM, about 250 RPM to about 750 RPM, about 250 RPM to about 700 RPM, about 250 RPM to about 650 RPM, about 250 RPM to about 600 RPM, about 250 RPM to about 550 RPM, about 250 RPM to about 500 RPM, about 250 RPM to about 450 RPM, about 250 RPM to about 400 RPM, about 250 RPM to about 350 RPM, about 250 RPM to about 300 RPM, about 300 RPM to about 800 RPM, about 300 RPM to about 750 RPM, about 300 RPM to about 700 RPM, about 300 RPM to about 650 RPM, about 300 RPM to about 600 RPM, about 300 RPM to about 550 RPM, about 300 RPM to about 500 RPM, about 300 RPM to about 450 RPM, about 300 RPM to about 400 RPM, about 300 RPM to about 350 RPM, about 350 RPM to about 800 RPM, about 350 RPM to about 750 RPM, about 350 RPM to about 700 RPM, about 350 RPM to about 650 RPM, about 350 RPM to about 600 RPM, about 350 RPM to about 550 RPM, about 350 RPM to about 500 RPM, about 350 RPM to about 450 RPM, about 350 RPM to about 400 RPM, about 400 RPM to about 800 RPM, about 400 RPM to about 750 RPM, about 400 RPM to about 700 RPM, about 400 RPM to about 650 RPM, about 400 RPM to about 600 RPM, about 400 RPM to about 550 RPM, about 400 RPM to about 500 RPM, about 400 RPM to about 450 RPM, about 450 RPM to about 800 RPM, about 450 RPM to about 750 RPM, about 450 RPM to about 700 RPM, about 450 RPM to about 650 RPM, about 450 RPM to about 600 RPM, about 450 RPM to about 550 RPM, about 450 RPM to about 500 RPM, about 500 RPM to about 800 RPM, about 500 RPM to about 750 RPM, about 500 RPM to about 700 RPM, about 500 RPM to about 650 RPM, about 500 RPM to about 600 RPM, about 500 RPM to about 550 RPM, about 550 RPM to about 800 RPM, about 550 RPM to about 750 RPM, about 550 RPM to about 700 RPM, about 550 RPM to about 650 RPM, about 550 RPM to about 600 RPM, about 600 RPM to about 800 RPM, about 600 RPM to about 750 RPM, about 600 RPM to about 700 RPM, about 600 RPM to about 650 RPM, about 650 RPM to about 800 RPM, about 650 RPM to about 750 RPM, about 650 RPM to about 700 RPM, about 700 RPM to about 800 RPM, about 700 RPM to about 750 RPM, or about 750 RPM to about 800 RPM.

### Humidity

In some examples, the culturing can be performed in a controlled humidified atmosphere (e.g., at a humidity of greater than 20%, 25%, 30%, 35%, 40%, 45%, 50%, 55% 60%, 65%, 70%, 75%, 80%, 85%, 90%, 95% or 100%).

### Recovering Recombinant Protein

In some embodiments, the recombinant protein is harvested from the recombinant protein production medium by removing, or otherwise physically separating the recombinant protein production medium from the recombinant bacteria. A variety of different methods for removing recombinant protein production medium from recombinant bacteria are known in the art, including, for example, centrifugation, filtration, pipetting, and/or aspiration. In some embodiments of any of the methods described herein, the method does not include a physical (e.g., homogenization, French press, glass or ceramic beads, sonication, autoclave, microwave, freeze/thawing, or mortar and pestle) or a chemical step (e.g., enzymes, e.g., such as cellulases, chitinases, lysozyme, mannase, or glycanase; an alcohol; chloroform; ether; or a chelator (e.g., EDTA)) to disrupt the outer membrane of the recombinant bacteria.

In some embodiments, the recombinant protein is recovered at a concentration in the recombinant protein production medium of about 10 mg/L to about 350 mg/L, about 10 mg/L to about 325 mg/L, about 10 mg/L to about 300 mg/L, about 10 mg/L to about 275 mg/L, about 10 mg/L to about 250 mg/L, about 10 mg/L to about 225 mg/L, about 10 mg/L to about 200 mg/L, about 10 mg/L to about 175 mg/L, about 10 mg/L to about 150 mg/L, about 10 mg/L to about 125 mg/L, about 10 mg/L to about 100 mg/L, about 10 mg/L to about 75 mg/L, about 10 mg/L to about 50 mg/L, about 10 mg/L to about 25 mg/L, about 25 mg/L to about 350 mg/L, about 25 mg/L to about 325 mg/L, about 25 mg/L to about 300 mg/L, about 25 mg/L to about 275 mg/L, about 25 mg/L to about 250 mg/L, about 25 mg/L to about 225 mg/L, about 25 mg/L to about 200 mg/L, about 25 mg/L to about 175 mg/L, about 25 mg/L to about 150 mg/L, about 25 mg/L to about 125 mg/L, about 25 mg/L to about 100 mg/L, about 25 mg/L to about 75 mg/L, about 25 mg/L to about 50 mg/L, about 50 mg/L to about 350 mg/L, about 50 mg/L to about 325 mg/L, about 50 mg/L to about 300 mg/L, about 50 mg/L to about 275 mg/L, about 50 mg/L to about 250 mg/L, about 50 mg/L to about 225 mg/L, about 50 mg/L to about 200 mg/L, about 50 mg/L to about 175 mg/L, about 50 mg/L to about 150 mg/L, about 50 mg/L to about 125 mg/L, about 50 mg/L to about 100 mg/L, about 50 mg/L to about 75 mg/L, about 75 mg/L to about 350 mg/L, about 75 mg/L to about 325 mg/L, about 75 mg/L to about 300 mg/L, about 75 mg/L to about 275 mg/L, about 75 mg/L to about 250 mg/L, about 75 mg/L to about 225 mg/L, about 75 mg/L to about 200 mg/L, about 75 mg/L to about 175 mg/L, about 75 mg/L to about 150 mg/L, about 75 mg/L to about 125 mg/L, about 75 mg/L to about 100 mg/L, about 100 mg/L to about 350 mg/L, about 100 mg/L to about 325 mg/L, about 100 mg/L to about 300 mg/L, about 100 mg/L to about 275 mg/L, about 100 mg/L to about 250 mg/L, about 100 mg/L to about 225 mg/L, about 100 mg/L to about 200 mg/L, about 100 mg/L to about 175 mg/L, about 100 mg/L to about 150 mg/L, about 100 mg/L to about 125 mg/L, about 125 mg/L to about 350 mg/L, about 125 mg/L to about 325 mg/L, about 125 mg/L to about 300 mg/L, about 125 mg/L to about 275 mg/L, about 125 mg/L to about 250 mg/L, about 125 mg/L to about 225 mg/L, about 125 mg/L to about 200 mg/L, about 125 mg/L to about 175 mg/L, about 125 mg/L to about 150 mg/L, about 150 mg/L to about 350 mg/L, about 150 mg/L to about 325 mg/L, about 150 mg/L to about 300 mg/L, about 150 mg/L to about 275 mg/L, about 150 mg/L to about 250 mg/L, about 150 mg/L to about 225 mg/L, about 150 mg/L to about 200 mg/L, about 150 mg/L to about 175 mg/L, about 175 mg/L to about 350 mg/L, about 175 mg/L to about 325 mg/L, about 175 mg/L to about 300 mg/L, about 175 mg/L to about 275 mg/L, about 175 mg/L to about 250 mg/L, about 175 mg/L to about 225 mg/L, about 175 mg/L to about 200 mg/L, about 200 mg/L to about 350 mg/L, about 200 mg/L to about 325 mg/L, about 200 mg/L to about 300 mg/L, about 200 mg/L to about 275 mg/L, about 200 mg/L to about 250 mg/L, about 200 mg/L to about 225 mg/L, about 225 mg/L to about 350 mg/L, about 225 mg/L to about 325 mg/L, about 225 mg/L to about 300 mg/L, about 225 mg/L to about 275 mg/L, about 225 mg/L to about 250 mg/L, about 250 mg/L to about 350 mg/L, about 250 mg/L to about 325 mg/L, about 250 mg/L to about 300 mg/L, about 250 mg/L to about 275 mg/L, about 275 mg/L to about 350 mg/L, about 275 mg/L to about 325 mg/L, about 275 mg/L to about 300 mg/L, about 300 mg/L to about 350 mg/L, about 300 mg/L to about 325 mg/L, or about 335 mg/L to about 350 mg/L (e.g., in a method that does not include a physical or a chemical step to disrupt the outer membrane of the recombinant bacteria).

In one embodiment of the present invention, the cell culturing is conducted in a fermenter with a starting recombinant protein production medium weight of 1,100 kg, the culture medium comprises 30 mM of phosphate and 1.3 g/L MgSO₄, the total EFT is 28 hours, the max OD₆₀₀ is 130, and the titer of the recombinant protein (e.g. ranibizumab) in the medium is 70-90 mg/L.

In another embodiment of the present invention, the cell culturing is conducted in a fermenter with a starting recombinant protein production medium weight of 600 kg, the culture medium comprises 60 mM of phosphate and 2.3 g/L MgSO₄, the total EFT is 48-50 hours, the max OD₆₀₀ is about 180, and the titer of the recombinant protein (e.g. ranibizumab) in the medium is 200-250 mg/L.

### Purifying Recombinant Protein

Some embodiments of these methods further include purifying the recombinant protein from the recombinant protein production medium (e.g., a recombinant protein production medium that is substantially free of recombinant bacteria) or a diluted recombinant protein production medium (e.g., a diluted recombinant protein production medium that is substantially free of recombinant bacteria). In some examples, purifying the recombinant protein includes the performance of one or more of: filtration, capturing, purification, and polishing.

### Filtering

In some embodiments, the method of purifying the recombinant protein can include, at least in part, a step of filtering a liquid containing the recombinant protein. The filtration can be performed, e.g., using a filter, or a chromatography column or chromatographic membrane that contains a molecule sieve resin.

### Capturing

In some embodiments, a recombinant protein can be purified using, at least in part, a step of capturing a recombinant protein from a liquid. Capturing a recombinant protein can include the use of, e.g., a chromatography column or a chromatography resin, e.g., that utilizes a capture mechanism. Non-limiting examples of capturing mechanisms include an antibody- or antibody fragment-binding capture mechanism, an aptamer-binding capture mechanism, a protein L-binding capture mechanism, a substrate-binding capture mechanism, and a cofactor-binding capture mechanism. For example, if the recombinant protein is an antibody, or an antigen-binding fragment, the capturing mechanism can be a protein L- or a protein A-binding capture mechanism or an antigen-binding capturing mechanism (wherein the capturing antigen is specifically recognized by the recombinant antibody or recombinant antigen-binding fragment). Non-limiting resins that capture a recombinant protein are described in the art. In order to capture the recombinant protein using the chromatography column or chromatographic membrane, one typically performs the sequential chromatographic steps of loading, washing, eluting, and regenerating the chromatography column or chromatography membrane. As one of skill in the art can appreciate, the flow rates and buffers to be used loading, washing, eluting, and regenerating steps are chosen based on the chemical properties of the recombinant protein.

### Purifying

In some embodiments, the methods of purifying a recombinant protein can include a step of purifying a recombinant protein using a chromatography column or chromatographic membrane that contains a resin, e.g., that binds the recombinant protein. For example, the chromatography column or chromatography membrane can be used to perform cation exchange or anion exchange chromatography, or molecular sieve chromatography. Exemplary resins that can be used to purify a recombinant protein are known in the art.

As can be appreciated by one skilled in the art, the step of purifying a recombinant protein can, e.g., include the steps of loading, washing, eluting, and equilibrating the at least one chromatography column or chromatographic membrane used to perform the unit of operation of purifying the recombinant protein. Typically, the elution buffer coming out of a chromatography column or chromatographic membrane used to perform the unit operation of purifying contains the recombinant protein.

Following the loading of the recombinant protein onto the chromatographic column or chromatographic membrane that is used to perform the unit operation of purifying the recombinant protein, the chromatographic column or chromatographic membrane is washed with at least one washing buffer. As can be appreciated in the art, the washing buffer is meant to elute all proteins that are not the recombinant protein from the chromatography column or chromatographic membrane, while not disturbing the interaction of the recombinant protein with the resin or otherwise eluting the recombinant protein.

Non-limiting examples of elution buffers used in these methods will depend on the resin and/or the recombinant protein. For example, an elution buffer can contain a different concentration of salt (e.g., increased salt concentration), a different pH (e.g., an increased or decreased pH), or a molecule that will compete with the recombinant protein for binding to the resin. Examples of such elution buffers are known in the art.

Following the elution of the recombinant protein from the chromatographic column or chromatographic membrane used to perform the unit operation of purifying the recombinant protein, the chromatography column or chromatographic membrane can be equilibrated using a regeneration buffer.

### Polishing

In some embodiments, the methods of purifying a recombinant protein can, at least in part, include a step polishing the recombinant protein. A recombinant protein can be polished using, e.g., a chromatography column or chromatographic membrane that contains a resin, e.g., cation exchange, anion exchange, or molecular sieve chromatography. Non-limiting examples of resins used to polish a recombinant protein are known in the art.

As can be appreciated in the art, polishing a recombinant protein using the chromatography column or chromatography membrane can include, e.g., the steps of loading, chasing, and regenerating the chromatography column or chromatographic membrane. For example, when the steps of loading, chasing, and regenerating are used to perform the polishing, the recombinant protein does not bind the resin in the chromatography column or chromatography membrane, and the recombinant protein is eluted from the chromatography column or chromatographic membrane in the loading and chasing steps, and the regenerating step is used to remove any impurities from the chromatography column or chromatographic membrane. Examples of flow rates and buffer volumes to be used in each of the loading, chasing, and regenerating steps are known in the art.

### Pharmaceutical Compositions

Also provided herein are pharmaceutical compositions that include at least one (e.g., at least two, at least three, at least four or at least five) of the recombinant proteins produced using any of the methods provided herein. In some embodiments, two or more (e.g., two, three, four or five) of any of the recombinant proteins described herein are present in a pharmaceutical composition in any combination.

The pharmaceutical compositions may be formulated in any manner described in the art. Pharmaceutical compositions are formulated to be compatible with their intended route of administration (e.g., intraarterial, intradermal, intramuscular, intravenous, intraperitoneal, or subcutaneous), and in dosage unit form (i.e., physically discrete units containing a predetermined quantity of recombinant protein for ease of administration and uniformity of dosage). The pharmaceutical compositions can include a sterile diluent (e.g., sterile water, saline, or water for injection), a fixed oil, polyethylene glycol, glycerin, propylene glycol, or other synthetic solvents, antibacterial or antifungal agents (e.g., benzyl alcohol, methyl parabens, chlorobutanol, phenol, ascorbic acid, or thimerosal), antioxidants (e.g., ascorbic acid or sodium bisulfite), chelating agents (e.g., ethylenediaminetetraacetic acid (EDTA)), buffers (e.g., acetate buffer, citrate buffer, or phosphate buffer), isotonic agents (e.g., sugars, polyalcohols (e.g., mannitol or sorbitol), or salts (e.g., sodium chloride), or any combination thereof.

Pharmaceutical compositions that include any of the recombinant proteins produced by any of the methods described herein (e.g., ranibizumab) can include about 5 mg/mL to about 10 mg/mL, about 5 mg/mL to about 9 mg/mL, about 5 mg/mL to about 8 mg/mL, about 5 mg/mL to about 7 mg/mL, about 5 mg/mL to about 6 mg/mL, about 6 mg/mL to about 10 mg/mL, about 6 mg/mL to about 9 mg/mL, about 6 mg/mL to about 8 mg/mL, about 6 mg/mL to about 7 mg/mL, about 7 mg/mL to about 10 mg/mL, about 7 mg/mL to about 9 mg/mL, about 7 mg/mL to about 8 mg/mL, about 8 mg/mL to about 10 mg/mL, about 8 mg/mL to about 9 mg/mL, or about 9 mg/mL to about 10 mg/mL, of the recombinant protein produced by any of the methods described herein (e.g., ranibizumab). Some embodiments of any of the pharmaceutical compositions described herein can further include a tonicity agent (e.g., α,α-trehalose dihydrate), a buffer (e.g., a histidine buffer), a surfactant (e.g., polysorbate 20), and water for injection, and have a pH of about 5.0 to about 7.5, about 5.0 to about 7.0, about 5.0 to about 6.5, about 5.0 to about 6.0, about 5.0 to about 5.5, about 5.5 to about 7.5, about 5.5 to about 7.0, about 5.5 to about 6.5, about 5.5 to about 6.0, about 6.0 to about 7.5, about 6.0 to about 7.0, about 6.0 to about 6.5, about 6.5 to about 7.5, about 6.5 to about 7.0, or about 7.0 to about 7.5. In some embodiments, any of the pharmaceutical compositions provided herein can be disposed in a glass vial (e.g., a single-use type I glass vial) or a syringe.

In some embodiments, any of the pharmaceutical compositions described herein include less than 100 ppm, less than 95 ppm, less than 90 ppm, less than 80 ppm, less than 70 ppm, less than 60 ppm, less than 50 ppm, less than 45 ppm, less than 40 ppm, less than 35 ppm, less than 30 ppm, less than 25 ppm, less than 20 ppm, less than 15 ppm, less than 10 ppm, or less than 5 ppm host cell protein.

### Methods of Treatment

Also provided herein are methods of treating a subject (e.g., a human) in need thereof that include administering to the subject a therapeutically effective amount of any of the pharmaceutical compositions described herein that include any of the recombinant proteins produced by any of the methods described herein. In some embodiments of these methods, the subject has been identified or diagnosed as having wet age-related macular degeneration, diabetic macular edema, or macular edema following retinal vein occlusion (e.g., branch retinal vein occlusion or central retinal vein occlusion).

Some embodiments include administration of one or more doses of any of the pharmaceutical compositions described herein to the subject (e.g., two or more doses, four or more doses, six or more doses, eight or more doses, or ten or more doses).

In some embodiments of any of the methods described herein, the pharmaceutical composition can be administered by subcutaneous administration, intramuscular administration, intravenous administration, intraocular administration, intraarterial administration, or intraperitoneal administration.

### Kits

Also provided herein are kits that include at least one dose of any of the pharmaceutical compositions described herein. In some embodiments, the kits can further include an item for use in administering a pharmaceutical composition (e.g., any of the pharmaceutical compositions described herein) to the mammal (e.g., a human).

In some examples, the kit can further include a sterile glass vial, where the pharmaceutical composition is disposed within the sterile glass vial. In some embodiments, the kit can further include a syringe and the pharmaceutical composition is diposed within the syringe.

Some examples of the kits include one or more doses (e.g., at least two, at least three, at least four, at least five, at least six, at least seven, at least eight, at least nine, at least ten, at least eleven, at least twelve, at least thirteen, at least fourteen, at least twenty, at least thirty, at least forty, or at least fifty doses) (e.g., intravenous, intraperitoneal, subcutaneous, intramuscular, or intraocular doses) of any of the pharmaceutical compositions described herein. In some examples, the kit further includes instructions for administering the pharmaceutical composition (or a dose of the pharmaceutical composition) to a mammal (e.g., a human in need thereof, e.g., any of the exemplary subjects described herein).

Also included herein are kits that include a sterile vial of a lyophilized recombinant protein cake or powder, instructions for reconstituting the cake or powder, and instructions for administration (e.g., intravenous, intramuscular, intraperitoneal, intraarterial, or intraocular administration) of the reconstituted solution to a mammal (e.g., a human in need thereof or any of the exemplary subjects described herein).

In some embodiments, the kits include a pharmaceutical composition including at least one of the recombinant proteins produced by any of the methods described herein, and a composition including at least one additional therapeutic agent. In some embodiments, the kit further contains instructions for administering the pharmaceutical composition including the at least one of the recombinant proteins produced by any of the methods described herein and a composition including at least one additional therapeutic agent to a mammal (e.g., a human in need thereof or any of the other exemplary subjects described herein).

### EXAMPLES

The disclosure is further described in the following examples, which do not limit the scope of the disclosure described in the claims.

### Example 1. Testing of phoA Promoter for Expression of Ranibizumab

### Seed Culture

The contents of a frozen E. coli BL21 phoA322 was transferred into a sterile 1 L baffled shake flask containing 250 mL of sterile seed medium (20.92 g/L (MOPS), 1.30 g/L K₂HPO₄, 3.36 g/L KH₂PO₄, 23.88 g/L yeast extract, 3.88 g/L ammonium sulfate, 0.72 g/L citric acid, 0.19 g/L FeCl₃-6H₂O, 4.50 g/L glucose, 0.65 g/L MgSO₄-7H₂O, 50.0 mg/L carbenicillin). The flask was placed into a shaker incubator at 30° C. and rotated at 200 RPM.

### Production Reactor

Calibrated pH and dO₂ probes were placed into a 6.6 L Bioflo3000 bioreactor, and the reactor was batched with a batching fermentation medium (1.30 g/L K₂HPO₄, 3.35 g/L KH₂PO₄, 23.87 g/L yeast extract, 3.87 g/L ammonium sulfate, 0.71 g/L FeCl₃-6H₂O, and 0.20 g/L antifoam 204). The reactor was sterilized for 40 minutes at 122° C.±1° C. as shown in FIG. 7. Once the reactor was cool, glucose and magnesium sulfate were aseptically added. After sampling, the carbenicillin was added to the reactor to give the complete medium (2.0 g/L glucose, 0.65 g/L MgSO₄-7H₂O, and 50.0 mg/L carbenicillin).

### Inoculation and Growth

After 13.25 hours of incubation, the seed flask had reached an QD600 of 8.6. A portion of the culture, 75 mL, was used to inoculate the bioreactor. Data collection and the pH control were hooked up started at this time. The production reactor was then allowed to continue, sampling periodically (Table 1). At 5 hours, the glucose feed (726.0 g/L glucose monohydrate) was started. The glucose was fed exponentially for 8 hours using the equation below.

Feed Rate (L/h)=(CDW*V*μₘₐₓe\hat{ }μₘₐₓ*t)(Feed Conc.*Cell Yield)

Parameters used: CDW=3 g/L; V=3 L; μₘₐₓ=0.20; Feed Conc.=600 g/L; Cell Yield=0.45 g/g; and t=elapsed fermentation time (EFT).

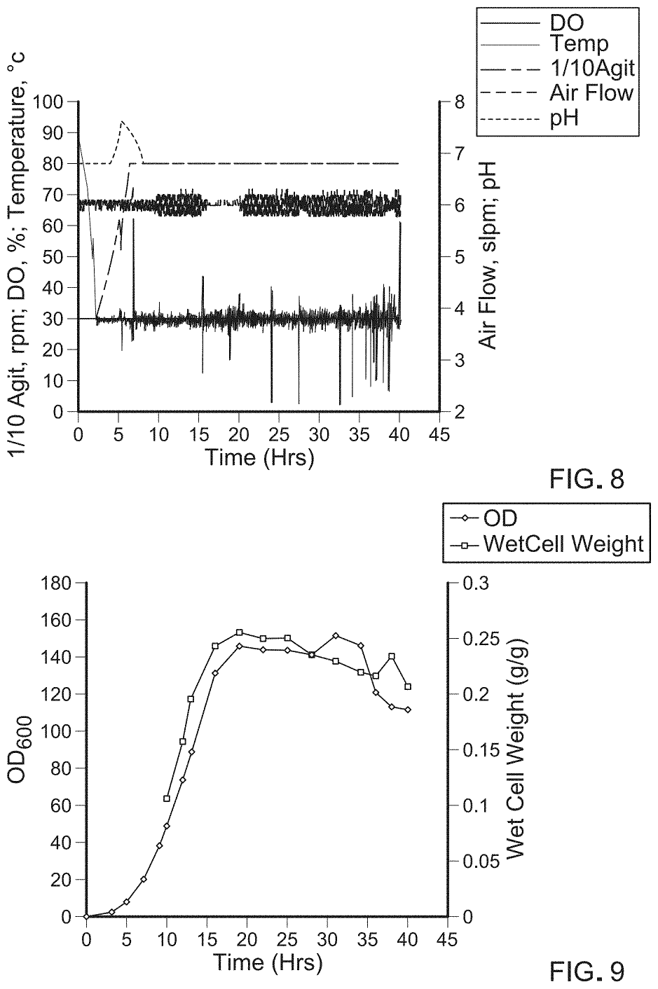

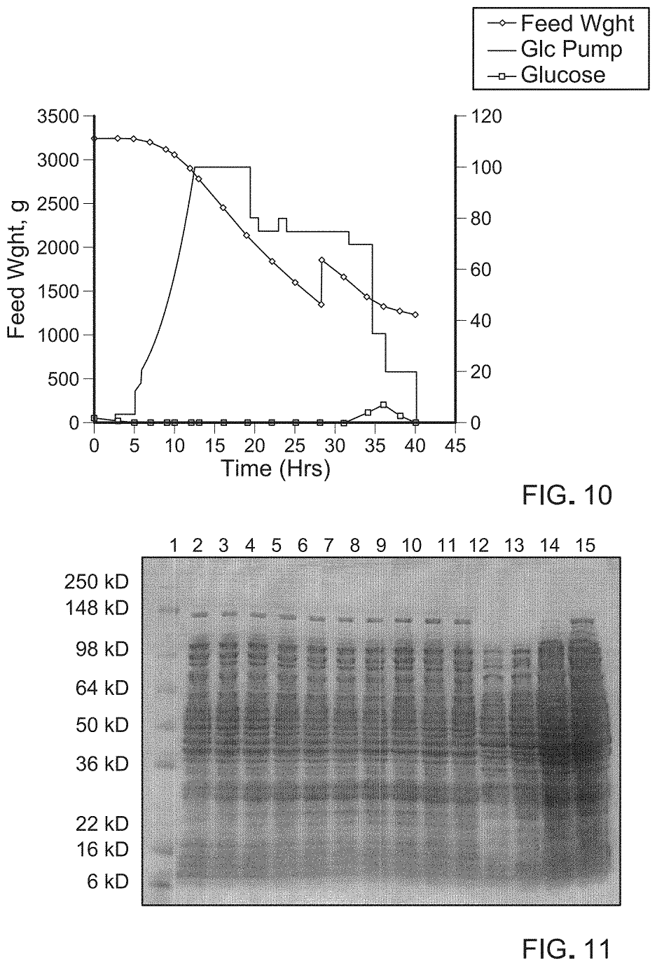

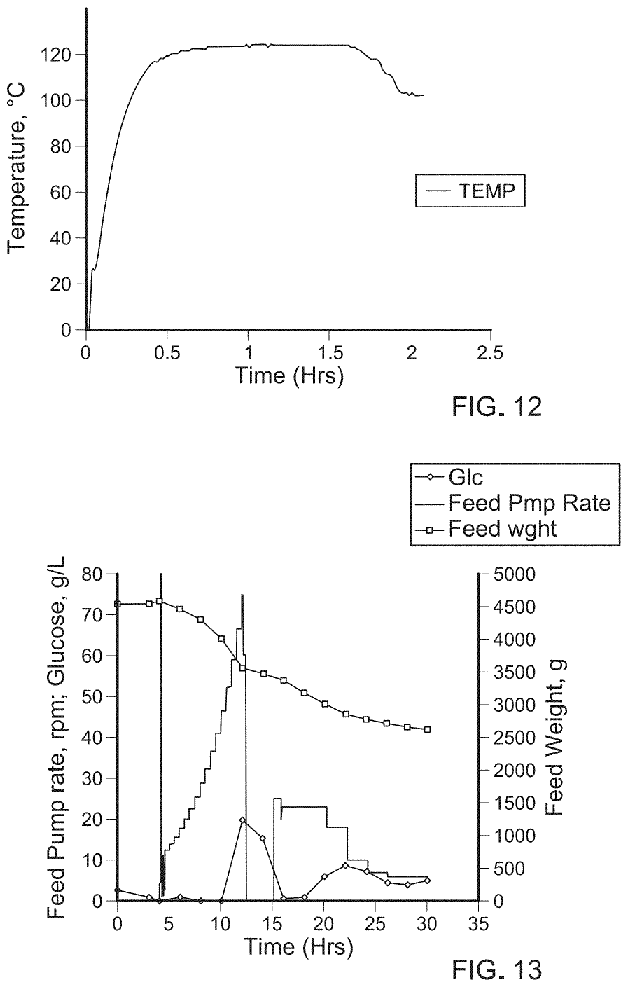

At 10 hours the OD₆₀₀ had reached 49.9, and the phosphate/magnesium feed (4.6 g/L sodium citrate dehydrate, 1.6 g/L FeCl₃-6H₂O, 18.26 g/L phosphoric acid (85%), and 6.5 g/L MgSO₄-7H₂O) was started at this time with a target rate of 1.4 t/L/hr until the end of the run. At 13 hours EFT, the glucose feed was set to a DO stat, adjusting step-wise if necessary throughout the run keeping glucose limiting in the culture by testing with the YSI. The data for the run are shown in Table 1 and FIGS. 8-10.

TABLE 1
Sample Points for COH04616

| Time | | | Glucose | |
|---|---|---|---|---|
| (h) | OD600 | WCW (g/g) | Conc. (g/L) | Comments |
| 0 | 0.44 | NA | 2.08 | Inoculate |
| 3 | 2.91 | NA | 1.05 | |
| 5 | 8.29 | NA | 0.004 | Begin glucose feed |
| 7 | 20.9 | NA | 0.005 | |
| 9 | 38.9 | NA | 0.002 | Begin phosphate feed |
| 10 | 49.9 | 0.1079 | 0.004 | |
| 12 | 74.9 | 0.1593 | 0.007 | |
| 13 | 90 | 0.1975 | 0.009 | |
| 16 | 132 | 0.2456 | 0.013 | |
| 19 | 147 | 0.257 | 0.013 | |
| 22 | 145 | 0.2519 | 0.020 | |
| 25 | 145 | 0.2526 | 0.017 | |
| 28 | 143 | 0.2375 | 0.024 | |
| 31 | 153 | 0.2319 | 0.059 | |
| 34 | 148 | 0.2225 | 4.24 | |
| 36 | 123 | 0.2193 | 7.36 | |
| 38 | 115 | 0.2368 | 2.98 | |
| 40 | 114 | 0.2091 | 0.020 | Cool for harvest |

NA, Not assayed

### Harvesting of Product

After 40 hours of growth, the OD₆₀₀ had reached 114 and the production reactor was cooled down to <20° C. for harvest. Microscopic examination of the cells revealed no visible inclusions. The cell paste was collected by centrifugation with Sorvall centrifuge by spinning at 10,000×g for 15 minutes. The supernatant was also collected and frozen at −20° C. The cell paste was frozen in liquid nitrogen and placed in a heat-seal bag, with a weight of 1355.1 g.

A total of 178.5 g of ammonium hydroxide was used for pH control. A total of 2567.8 g of glucose feed, 163 g of phosphate/magnesium feed were fed during the run. Final weight of the culture from the reactor was 4830 g.

Protein expression was determined by running the pre-induction and post-induction samples on a gradient 4-20% SDS gel after lysis of the whole cells under reduced conditions (FIG. 11). The gel did not reveal any new bands after 16 hours.

### Example 2. Testing 2× Magnesium with phoA Promoter for Expression of Ranibizumab

### Seed Culture

The contents of a frozen E. coli BL21 phoA322 were transferred into a sterile 1 L baffled shake flask containing 250 mL of sterile seed medium (20.92 g/L (MOPS), 1.30 g/L K₂HPO₄, 3.36 g/L KH₂PO₄, 23.88 g/L yeast extract, 3.88 g/L ammonium sulfate, 0.72 g/L citric acid, 0.19 g/L FeCl₃-6H₂O, and 10 mg/L tetracycline). The flask was placed into a shaker incubator at 30° C. and rotated at 200 RPM.

### Production Reactor

Calibrated pH and dO₂ probes were placed into a 6.6 L Bioflo3000 bioreactor, and the reactor was batched with a batching fermentation medium (same as described in Example 1). The reactor was sterilized for 40 minutes at 122° C.±1° C. as shown in FIG. 13. Once the reactor was cool, glucose and magnesium sulfate were aseptically added. After sampling, the tetracycline was added to the reactor to give the complete medium (2.25 g/L glucose, 1.3 g/L MgSO₄-7H₂O, and 10.0 mg/L tetracycline).

### Inoculation and Growth

After 13 hours of incubation, the seed flask had reached an QD₆₀₀ of 16.2. A portion of the culture, 75 mL, was used to inoculate the bioreactor. Data collection and the pH control were started at this time. The production reactor was then allowed to continue, sampling periodically (Table 2). At 5 hours, the glucose feed (726.0 g/L glucose monohydrate) was started. The glucose was fed using a step feed program for 8 hours. Glucose was monitored by using a YSI to analyze the supernatants from each time point. The levels and pump rate are shown in FIG. 13. Due to the slow growth of this culture, the glucose feed rate was decreased as necessary to prevent glucose build up in the reactor (FIG. 13).

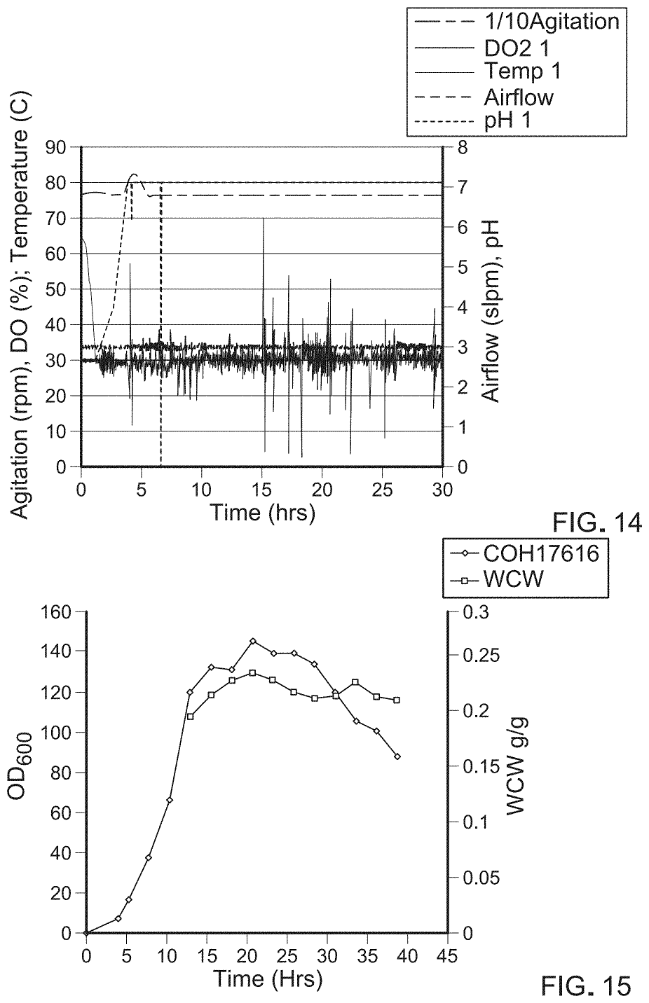

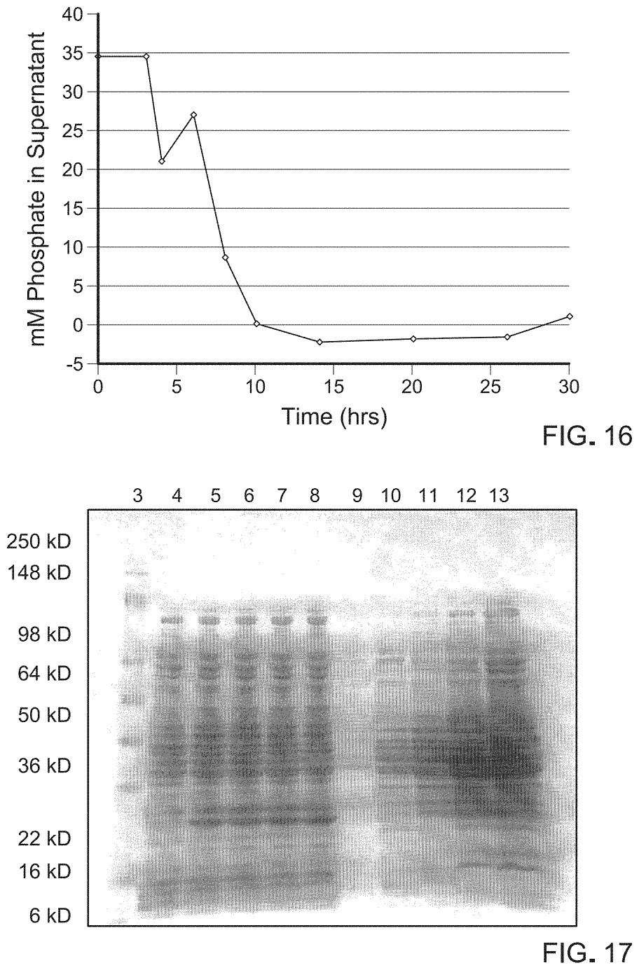

At 9 hours the OD₆₀₀ had reached 51.0, and the phosphate/magnesium feed (4.6 g/L sodium citrate dehydrate, 1.6 g/L FeCl₃-6H₂O, 18.26 g/L phosphoric acid (85%), and 6.5 g/L MgSO₄-7H₂O) was started at this time with a target rate of 1.8 g/L/hr until the end of the run. At 13 hours EFT, the glucose feed was set to a linear rate, adjusting step-wise if necessary throughout the run keeping glucose limiting in the culture by testing with the YSI. The data for the run are shown in Table 2 and FIGS. 13-16.

TABLE 2
Sample points for COH17616

| Time | | | Glucose | |
|---|---|---|---|---|
| (h) | OD600 | WCW (g/g) | Conc. (g/L) | Comments |
| 0 | 0.61 | NA | 2.73 | Inoculate |
| 3 | 7.30 | NA | 1.07 | |
| 4 | 16.3 | NA | 0.046 | Begin glucose feed |
| 6 | 37.9 | NA | 1.01 | |
| 8 | 67.3 | NA | 0.082 | Begin phosphate feed |
| 10 | 121 | 0.2033 | 0.141 | |
| 12 | 133 | 0.2239 | 19.8 | |
| 14 | 132 | 0.2372 | 15.5 | |
| 16 | 146 | 0.2444 | 0.643 | |
| 18 | 140 | 0.238 | 1.04 | |
| 20 | 140 | 0.2271 | 6.14 | |
| 22 | 135 | 0.2212 | 9.05 | |
| 24 | 121 | 0.2232 | 7.56 | |
| 26 | 107 | 0.2365 | 4.80 | |
| 28 | 102 | 0.2226 | 4.08 | |
| 30 | 89 | 0.2192 | 5.38 | Cool for harvest |

NA, Not assayed

### Harvesting of Product

After 34.5 hours of growth, the OD₆₀₀ had reached 165 and the production reactor was cooled down to <20° C. for harvest. Microscopic examination of the cells revealed no visible inclusions. The cell paste was collected by centrifugation with Sorvall centrifuge by spinning at 10,000×g for 15 minutes. The supernatant was also collected and saved. A total of 3269 g of supernatant was collected. The supernatant was brought to 5 mM EDTA by the addition of a 500 mM EDTA solution.

A total of 166.1 g of ammonium hydroxide were used for pH control this run. A total of 1920.5 g of glucose feed, 120.5 g of phosphate/magnesium feed were fed during the run. Final weight of the culture from the reactor was 4346 g.

Protein expression was determined by running the pre-induction and post-induction samples on a gradient 4-20% SDS gel after lysis of the whole cells under reduced conditions (FIG. 17). The gel revealed an induction band in the pellet samples in the range of 25-30 kDa after 16 hours.

### Example 3. Testing ⅓ Magnesium with phOA Promoter for Expression of Ranibizumab

### Seed Culture

The contents of a frozen E. coli BL21 phoA322 was transferred into a sterile 1 L baffled shake flask containing 250 mL of sterile seed medium (as described in Example 2). The flask was placed into a shaker incubator at 30° C. and rotated at 200 RPM.

### Production Reactor

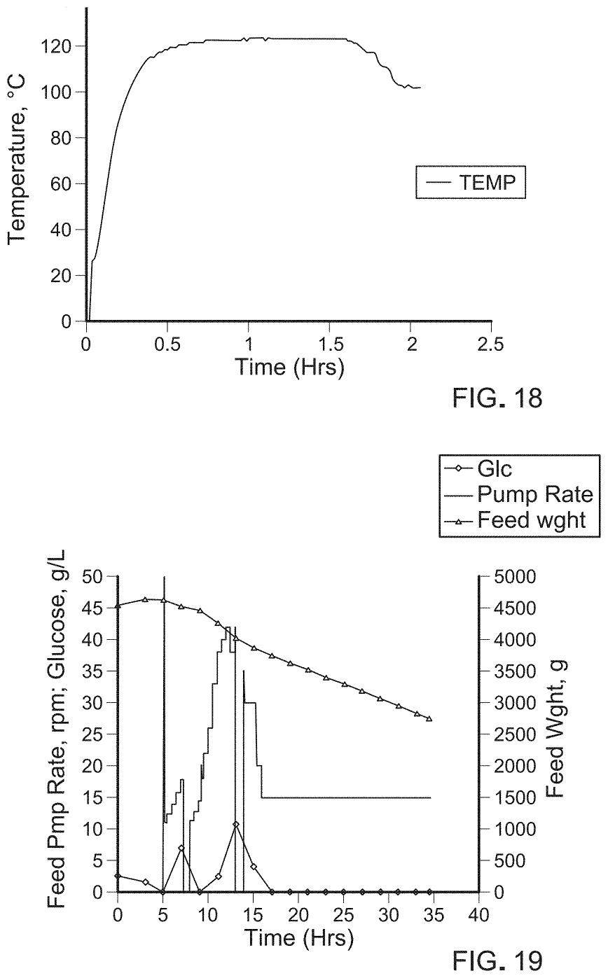

Calibrated pH and dO₂ probes were placed into a 6.6 L Bioflo3000 bioreactor, and the reactor was batched with a batching fermentation medium (1.30 g/L K₂HPO₄, 3.35 g/L KH₂PO₄, 23.85 g/L yeast extract, 3.85 g/L ammonium sulfate, 0.71 g/L FeCl₃-6H₂O, and 0.20 g/L antifoam 204). The reactor was sterilized for 40 minutes at 122° C.±1° C. as shown in FIG. 18. Once the reactor was cool, glucose and magnesium sulfate were aseptically added. Tetracycline was added to the reactor to give the complete medium (2.25 g/L glucose, 0.22 g/L MgSO₄-7H₂O, and 10.0 mg/L tetracycline).

### Inoculation and Growth

After 13 hours of incubation, the seed flask reached an QD₆₀₀ of 16.2. A portion of the culture, 75 mL, was used to inoculate the bioreactor. Data collection and the pH control were hooked up started at this time. The production reactor was then allowed to continue, sampling periodically (Table 3). At 5 hours, the glucose feed (726.0 g/L glucose monohydrate) was started. The glucose was fed using a step feed program for 8 hours. Glucose was monitored by using a YSI to analyze the supernatants from each time point. The levels and pump rate are shown in FIG. 19.

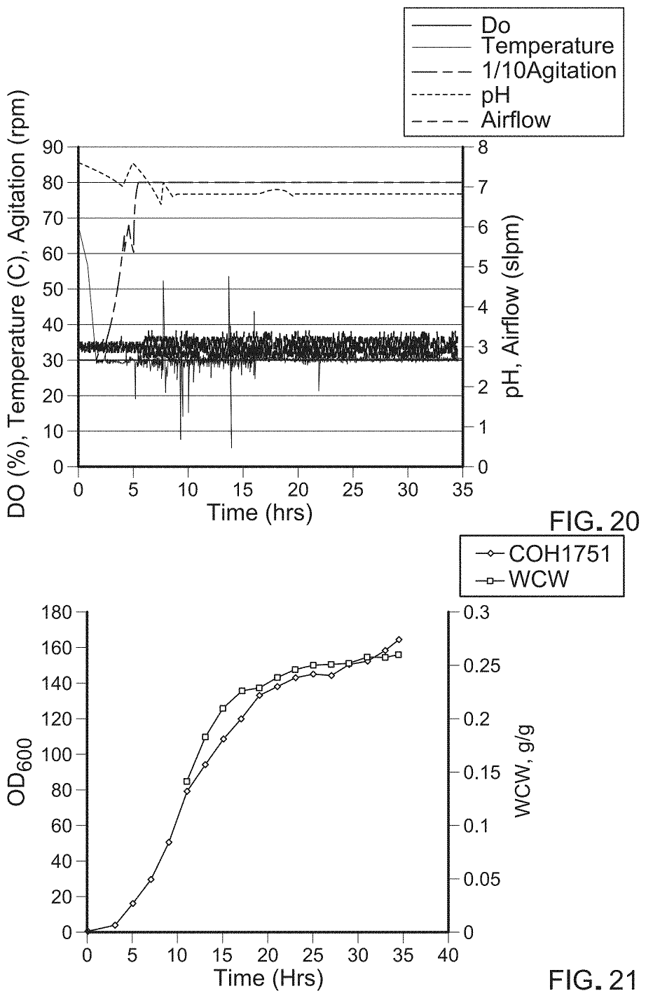

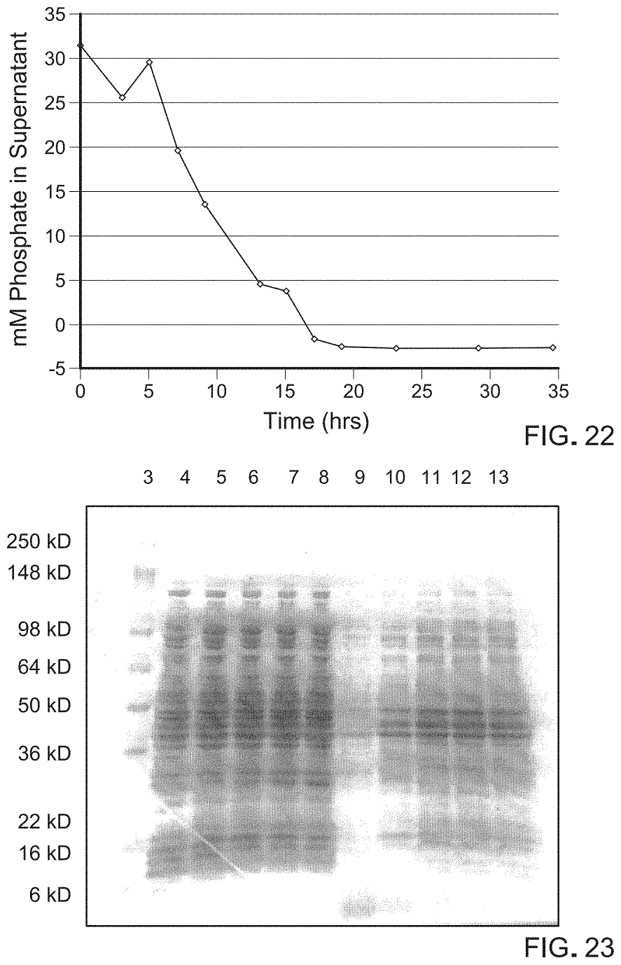

At 9 hours the OD₆₀₀ had reached 51.0, and the phosphate/magnesium feed (4.6 g/L sodium citrate dehydrate, 1.6 g/L FeCl₃-6H₂O, 18.26 g/L phosphoric acid (85%), and 6.5 g/L MgSO₄-7H₂O) was started at this time with a target rate of 1.8 g/L/hr until the end of the run. At 13 hours EFT, the glucose feed was set to a linear rate, adjusting step-wise if necessary throughout the run keeping glucose limiting in the culture by testing with the YSI. The data for the run are shown in Table 3 and FIGS. 19-22.

TABLE 3
Sample points for COH17516

| Time | | | Glucose | |
|---|---|---|---|---|
| (h) | OD600 | WCW (g/g) | Conc. (g/L) | Comments |
| 0 | 0.68 | NA | 2.65 | Inoculate |
| 3 | 4.06 | NA | 1.79 | |
| 5 | 16.1 | NA | 0.057 | Begin glucose feed |
| 7 | 30.1 | NA | 7.17 | |
| 9 | 51 | NA | 0.052 | Begin phosphate feed |
| 11 | 80 | 0.1436 | 2.29 | |
| 13 | 95 | 0.1853 | 10.8 | |
| 15 | 110 | 0.212 | 4.26 | |
| 17 | 121 | 0.2282 | 0.152 | |
| 19 | 134 | 0.2311 | 0.148 | |
| 21 | 139 | 0.2413 | 0.156 | |
| 23 | 144 | 0.2481 | 0.163 | |
| 25 | 146 | 0.2525 | 0.173 | |
| 27 | 145 | 0.253 | 0.177 | |
| 29 | 151 | 0.2538 | 0.167 | |
| 31 | 153 | 0.2597 | 0.160 | |
| 33 | 159 | 0.2594 | 0.138 | |
| 34.5 | 165 | 0.2619 | 0.121 | Cool for harvest |

NA, Not assayed

### Harvesting of Product

After 34.5 hours of growth, the OD₆₀₀ reached 165 and the production reactor was cooled down to <20° C. for harvest. Microscopic examination of the cells revealed no visible inclusions. The cell paste was collected by centrifugation with Sorvall centrifuge by spinning at 10,000×g for 15 minutes. The supernatant was also collected and saved. A total of 3135 g of supernatant was collected. The supernatant was brought to 5 mM EDTA by the addition of a 500 mM EDTA solution.

A total of 150.9 g of ammonium hydroxide was used for pH control. A total of 1875.7 g of glucose feed, 131.6 g of phosphate/magnesium feed were fed during the run. Final weight of the culture from the reactor was 4830 g.

Protein expression was determined by running the pre-induction and post-induction samples on a gradient 4-20% SDS gel after lysis of the whole cells under reduced conditions (FIG. 23). The gel revealed a weak induction band in the range of 25-30 kDa after 25 hours.

### Example 4. Effect of Magnesium Concentration on Secretion of Ranibizumab from Periplasm of Recombinant Bacteria

The effect of Mg²⁺ concentration on secretion of recombinant protein expressed by a microorganism was evaluated. In this experiment, E. coli BL21 phoA322 cells encoding ranibizumab were evaluated.

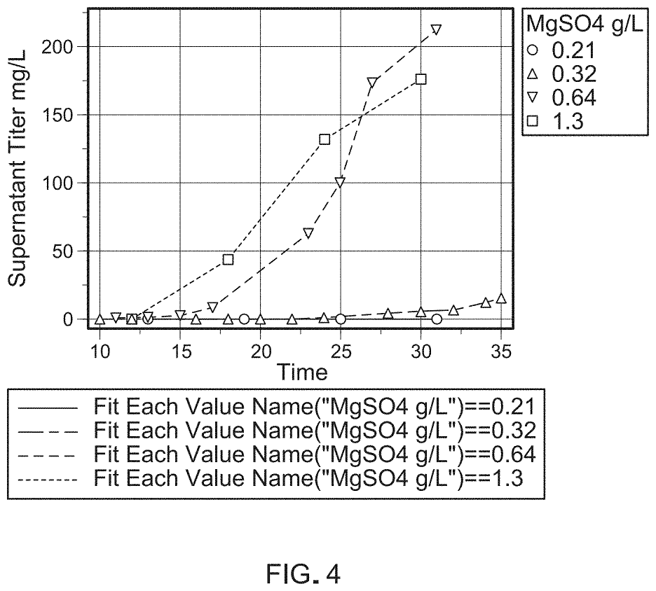

Ranibizumab culturing runs were conducted as described above with a batching fermentation medium comprising 0.21, 0.32, 0.64, or 1.3 g/L of MgSO₄. The amount of ranibizumab in the periplasm and the amount of ranibizumab secreted into the medium is graphed in FIGS. 3 and 4.

FIG. 3 shows ranibizumab titer in the periplasm over the duration of the runs. In runs with culture media with 0.21 and 0.32 g/L MgSO₄ the amount of ranibizumab in the periplasm increased over time without a significant drop off. Surprisingly, when the amount of MgSO₄ was increased to at least 0.64 g/L (e.g. 0.64 g/L, 1.3 g/L) the amount of ranibizumab in the periplasm increased much sooner in the run and then decreases without ever reach peak level again. The run with the highest amount of Mg²⁺ (1.3 g/L MgSO₄) in the culture media had the sharpest decrease in periplasm titer and a lower periplasm titer than the run with 0.64 g/L MgSO₄ in which the same result was seen, but a reduced degree.

FIG. 4 shows ranibizumab titer in the supernatant (culture medium) over the duration of the runs. In runs with culture media with 0.21 and 0.32 g/L MgSO₄ the amount of ranibizumab in the supernatant is flat over the duration of the run without a significant increase. Surprisingly, when the amount of MgSO₄ was increased to at least 0.64 g/L (e.g. 0.64 g/L, 1.3 g/L) the amount of ranibizumab in the supernatant increased much sooner in the run and continues to increase over the duration of the run. The run with the highest amount of Mg²⁺ (1.3 g/L MgSO₄) in the culture media had earlier increase in supernatant titer than the run with 0.64 g/L MgSO₄. The run with 0.64 g/L MgSO₄ also demonstrated a steep increase in supernatant titer, albeit later in the run.

The results graphed in FIGS. 3 and 4 show that secretion of a recombinant protein from the periplasm into the culture medium can be increased by Mg²⁺ in a dose dependent manner. It is noted that all runs were fed with a feed medium containing 6.5 g/L of MgSO₄. Since this value did not change among the runs, the feed of Mg²⁺ does not contribute to the effects seen in the runs with a high amount of Mg²⁺ in the culture medium as compared to the runs with a low amount of Mg²⁺ in the culture medium.

Thus, culturing a recombinant bacterium expressing a protein in liquid culture medium with at least about 0.64 g/L MgSO₄ results in the secretion of the protein into the culture medium.

### Example 5. Effect of Phosphate Concentration on Secretion of Ranibizumab from Periplasm of Recombinant Bacteria

In E. coli BL21 phoA322 cells phosphate starvation induces recombinant protein expression. To determine the effect of phosphate on secretion of a recombinant protein from the periplasm, culture media with high and low amounts of phosphate were evaluated.

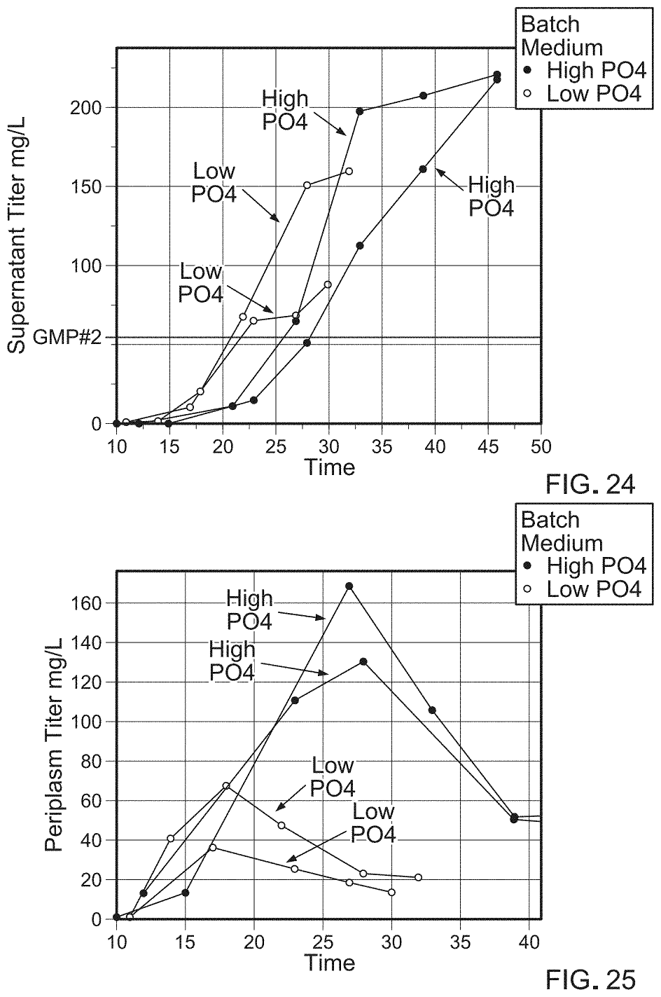

Ranibizumab culturing runs were conducted as described above with a batching fermentation medium comprising either a low amount of phosphate (1.30 g/L K₂HPO₄ and 3.35 g/L KH₂PO₄) or a high amount of phosphate (2.60 g/L K₂HPO₄ and 6.70 g/L KH₂PO₄). The amount of ranibizumab secreted into the medium, the amount of ranibizumab in the periplasm, and the OD₆₀₀ of the cell cultures is graphed in FIGS. 24-26.

FIG. 24 show ranibizumab secreted into the medium (supernatant titer) over the duration of the runs in culture media with low and high amounts of phosphate. Here, regardless of the amount of phosphate in the culture medium, ranibizumab was secreted into the medium. In media with higher phosphate a greater amount of ranibizumab was secreted into the medium over time.

FIG. 25 shows ranibizumab in the periplasm over the duration of the runs in culture media with low and high amounts of phosphate. Here, the amount of ranibizumab initially increases after induction but decreases after an inflection point in reached. This is indicative of the ranbizumab entering the periplasm then being subsequently secreted from the cell into culture medium. This pattern is the same regardless of the amount of phosphate present in the culture medium.

When FIGS. 24 and 25 are evaluated together it is clear that when the periplasm titer peaks during a cell culture run the rate at which ranibizumab is secreted into the culture medium increases. For the low phosphate runs the periplasm titer peaks around 17-18 hours EFT (FIG. 25) and the rate of secretion into the periplasm increases at the same time in FIG. 24. These results are consistent regardless of the amount of phosphate in the culture medium and are consistent with the Mg²⁺ dependent secretion observed in FIGS. 3-5.

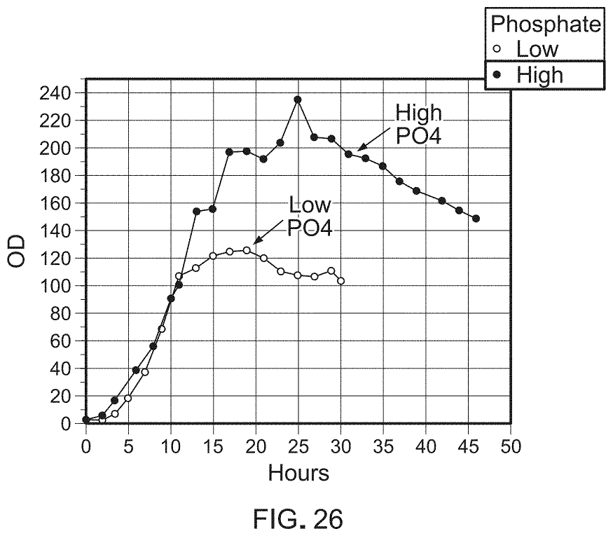

FIG. 26 shows that a higher cell density (OD₆₀₀) was obtained in the run with a culture medium containing a higher amount of phosphate. Since more phosphate was present which needed to be consumed by the cells prior to reaching phosphate starvation, exponential cell growth lasted longer resulting in more cells being produced. Once induction was triggered by phosphate starvation, the greater number of cells were able to survive longer and produce more recombinant protein. The high phosphate run was terminated after about 46 hours EFT while the low phosphate run was terminated after 30 hours EFT.

To determine the amount of ranibizumab yielded from the low and high phosphate runs, ranibizumab was harvested from the culture medium and purified. After purification, the low phosphate run had yield of 40 mg/L and high phosphate run had a yield of 100 mg/L.

The increase is cell density as a result of higher phosphate content in the culture medium results in the higher ranibizumab titers at first in the periplasm and later in the culture medium observed in FIGS. 24 and 25.

In another culturing run a batching fermentation medium comprising an even higher amount of phosphate (3.25 g/L K₂HPO₄ and 8.38 g/L KH₂PO₄) was evaluated. The run achieved an OD₆₀₀ of 197 at 16 hours EFT. The OD₆₀₀ values at 18, 20, 22, 24, 26, 28, 29, 30, and 32 hours EFT were 200, 206, 195, 200,188, 186, 200, 179, and 167, respectively. At 48 hours EFT, the OD₆₀₀ was 123 and the run was terminated. These results are consistent with high phosphate run described above (2.60 g/L K₂HPO₄ and 6.70 g/L KH₂PO₄) indicating that high amounts of phosphate in the culture medium can be used to produce and secrete recombinant proteins.

In conclusion, the amount of phosphate in the culture medium does not interfere with Mg²⁺ mediated secretion of recombinant protein into the culture medium. However, a higher amount of phosphate in the culture medium resulted in increased cell density. The higher number of cells in the high phosphate run were able to produce more protein which resulted in higher yield.

### Other Embodiments

It is to be understood that while the disclosure has been described in conjunction with the detailed description thereof, the foregoing description is intended to illustrate and not limit the scope, which is defined by the appended claims. Other aspects, advantages, and modifications are within the scope of the following claims.

1. A method of producing a recombinant protein, the method comprising:
providing a recombinant bacterium comprising a nucleic acid encoding a recombinant protein; and
culturing the recombinant bacterium in a liquid culture medium comprising about 0.3 mM to about 300 mM of Mg²⁺ under conditions sufficient for the production and release of the recombinant protein into the culture medium.

2. The method of claim 1, wherein the liquid culture medium comprises a magnesium salt.

3. The method of claim 2, wherein the magnesium salt is MgSO₄.

4. The method of claim 1, further comprising recovering the recombinant protein from the liquid culture medium.

5. The method of claim 4, wherein the recovered recombinant protein is at least 95% pure.

6. The method of claim 4, further comprising purifying the recovered recombinant protein.

7. The method of claim 6, further comprising formulating the purified recombinant protein.

8. The method of claim 7, wherein the method does not include the performance of more than two chromatography steps.

9. The method of claim 7, wherein the method does not include physical or chemical disruption of the outer membrane of the recombinant bacterium.

10. The method of claim 1, wherein the culturing is performed using a fermentor.

11. The method of claim 1, wherein the culturing is batch culturing.

12. The method of claim 1, wherein the culturing is fed batch culturing.

13. The method of claim 1, wherein the culturing comprises incubating the bacterium at a rotary agitation rate of about 80 revolutions per minute (RPM) to about 1000 RPM.

14. (canceled)

15. The method of claim 1, wherein the culturing is performed at about 30° C. to about 37° C.

16. The method of claim 1, wherein the recombinant bacterium is a Gram negative bacterium.

17. The method of claim 1, wherein the recombinant bacterium is selected from the group consisting of: K₁₂ E. coli bacterial cell, a Yersinia bacterial cell, a BL21 E. coli bacterial cell, a 60E4 E. coli, an Acinetobacter bacterial cell, a Bordella bacterial cell, a Brucella bacterial cell, a Cyanobacter bacterial cell, an Enterobacter bacterial cell, a Helicobacter bacterial cell, a Klebsiella bacterial cell, a Neisseria bacterial cell, a Pasteurella bacterial cell, a Pseudomonas bacterial cell, a Salmonella bacterial cell, and a Shigella bacterial cell.

18. The method of claim 1, wherein the nucleic acid encoding the recombinant protein is integrated into a chromosome of the recombinant bacterium.

19. The method of claim 1, wherein the nucleic acid encoding the recombinant protein is not integrated into a chromosome of the recombinant bacterium.

20. The method of claim 1, wherein the recombinant protein is an antibody or an antigen-binding antibody fragment.

21. The method of claim 1, wherein the recombinant protein is an antibody or an antigen-binding antibody fragment that specifically binds to human vascular endothelial growth factor A (VEGFA).

22. The method of claim 21, wherein the antigen-binding antibody fragment is ranibizumab.

23. The method of claim 1, wherein the nucleic acid encoding the recombinant protein is an expression vector.

24. The method of claim 23, wherein the nucleic acid encoding the recombinant protein comprises a sequence encoding a bacterial signal sequence.

25. The method of claim 1, wherein the liquid culture medium comprises about 0.4 mM to about 35 mM Mg²⁺.

26.-29. (canceled)

30. A recombinant protein produced by the method of claim 1.

31. A compositions comprising the recombinant protein of claim 30.

32. A pharmaceutical composition comprising a therapeutically effective amount of the recombinant protein of claim 30.

33. The pharmaceutical composition of claim 32, wherein the recombinant protein is ranibizumab.

34. The pharmaceutical composition of claim 33, wherein the pharmaceutical composition comprises about 5 mg/mL to about 10 mg/mL ranibizumab.

35. The pharmaceutical composition of claim 33, wherein the pharmaceutical composition further comprises a tonicity agent, a buffer, a surfactant, and water for injection, and wherein the pharmaceutical composition has a pH of about 5 to about 6.

36. The pharmaceutical composition of claim 35, wherein the pharmaceutical composition comprises α,α-trehalose dihydrate, a histidine buffer, polysorbate 20, and water for injection, and the pharmaceutical composition has a pH of about 5 to about 6.

37. A kit comprising the pharmaceutical composition of claim 32.

38. The kit of claim 37, further comprising a sterile glass vial, wherein the pharmaceutical composition is disposed within the sterile glass vial.

39. The kit of claim 37, further comprising a syringe, wherein the pharmaceutical compositions is disposed within the syringe.

40. A method of treating a subject in need thereof, the method comprising administering to the subject a therapeutically effective amount of the pharmaceutical composition of claim 32.

41. The method of claim 40, wherein the subject has been identified or diagnosed as having wet age-related macular degeneration, diabetic macular edema, or macular edema following retinal vein occlusion.

42. The method of claim 41, wherein the macular edema following retinal vein occlusion is branch retinal vein occlusion.

43. The method of claim 41, wherein the macular edema following retinal vein occlusion is central retinal vein occlusion.
